(19) United States
(12) Patent Application Publication
(10) Pub. No.: US 2024/0190828 A1
(43) Pub. Date: Jun. 13, 2024

(54) Small Molecule Inhibitors of PBRM1-BD2

(71) Applicant: The Medical College of Wisconsin, Inc., Milwaukee, WI (US)

(72) Inventors: Shifali Shishodia, Milwaukee, WI (US); Christopher J. Goetz, Wauwatosa, WI (US); Michael D. Olp, Milwaukee, WI (US); Brian Christopher Smith, Elm Grove, WI (US)

(21) Appl. No.: 18/546,143

(22) PCT Filed: Feb. 11, 2022
(86) PCT No.: PCT/US2022/016190

§ 371 Date: Aug. 11, 2023

Related U.S. Application Data
(60) Provisional application No. 63/148,356, filed on Feb. 11, 2021.
(60) Provisional application No. 63/279,610, filed on Nov. 15, 2021.

(51) Int. Cl. C07D 239/91; A61P 35/00; C07C 229/58; C07C 251/24; C07D 219/06; C07D 265/22

(52) U.S. Cl. CPC C07D 239/91; A61P 35/00; C07C 229/58; C07C 251/24; C07D 219/06; C07D 265/22

(57) ABSTRACT

The present disclosure provides novel inhibitors of PBRM1, specifically the 2^{nd} bromodomain of PBRM1 (PBRM1-BD2), pharmaceutical compositions and methods of use thereof.

### CROSS-REFERENCE TO RELATED APPLICATIONS

The present application claims priority to U.S. Provisional Patent Application No. 63/148,356, filed Feb. 11, 2021, and also claims priority to U.S. Provisional Patent Application No. 63/279,610, filed Nov. 15, 2021, the entire disclosures of each of which are incorporated by reference herein.

### STATEMENT REGARDING FEDERALLY SPONSORED RESEARCH

This invention was made with government support under R35 GM128840 awarded by the National Institutes of Health (NIH). The government has certain rights in the invention.

### BACKGROUND OF THE INVENTION

Bromodomain containing proteins PB1, SMARCA4, and SMARCA2 are important components of SWI/SNF chromatin remodeling complexes. Bromodomains are epigenetic readers of acetylated lysine residues on histones and other nuclear proteins. Bromodomains are found in at least 46 multidomain proteins within the human genome. Bromodomains regulate transcription through a variety of mechanisms and are implicated in various human diseases including cancer and other inflammatory disorders. Bromodomains are protein modules of ˜110 amino acids that recognize acetylated lysine (KAc) and share a common structure of four a helices linked by flexible loop regions and have been popular targets for chemical probe development. Selective small molecule inhibitors of the bromodomain and extraterminal domain (BET) family of bromodomain-containing proteins have progressed into clinical trials, but the remaining bromodomains have been largely unexplored clinically.

### SUMMARY OF THE INVENTION

The present invention describes novel inhibitors of PBRM1, specifically the 2^{nd} bromodomain of PBRM1 (PBRM1-BD2), and methods of use thereof.

The present invention provides, in one aspect, a compound of formula (I):

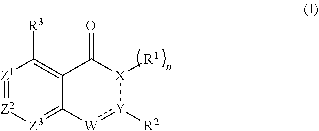

wherein X is selected from C, N, NH, and O;
   Y is selected from C and phenylene optionally substituted with halogen;
   W is N or NH;
   Z¹, Z², and Z³ are independently selected from CH and N;
   n is 0 or 1;
   R¹ is hydrogen, and R² is selected from the group consisting of carboxyl, pyridyl optionally substituted with one or more alkyl, and phenyl optionally substituted with one or more substituents selected from the group consisting of halogen, alkyl, alkoxy, and haloalkyl, or R¹ and R² together form a phenyl optionally substituted with one or more substituents selected from the group consisting of carboxyl and halogen; and
   R³ is selected from the group consisting of hydrogen, alkoxy, alkyl, and halogen.

In another aspect, the disclosure provides a compound of formula I(a):

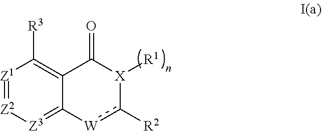

wherein X is N or O;
   W is N or NH;
   Z¹, Z², and Z³ are independently selected from CH and N;
   n is 0 or 1;
   R¹ is hydrogen;
   R² is selected from pyridyl optionally substituted with one or more alkyl, and phenyl optionally substituted with one or more substituents selected from the group consisting of halogen, alkyl, alkoxy, and haloalkyl; and
   R³ is selected from the group consisting of hydrogen, alkoxy, alkyl, and halogen.

In a further aspect, the disclosure provides a compound of formula I(b):

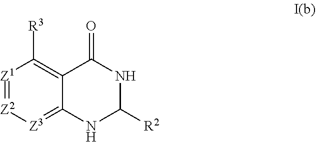

wherein Z¹, Z², and Z³ are independently selected from CH and N;
R² is selected from the group consisting of pyridyl optionally substituted with one or more alkyl and phenyl optionally substituted with one or more substituents selected from the group consisting of halogen, alkyl, alkoxy, and haloalkyl; and R³ is selected from the group consisting of hydrogen, alkoxy, alkyl, and halogen.

In yet another aspect, the disclosure provides a compound having the formula I(b), wherein R² is phenyl optionally substituted with one or more substituents selected from the group consisting of fluoro, chloro, methyl, methoxy, and trifluoromethyl; R³ is hydrogen, fluoro, chloro, bromo, or methyl; and Z¹, Z², and Z³ are CH.

In yet another aspect, the disclosure provides a compound that is

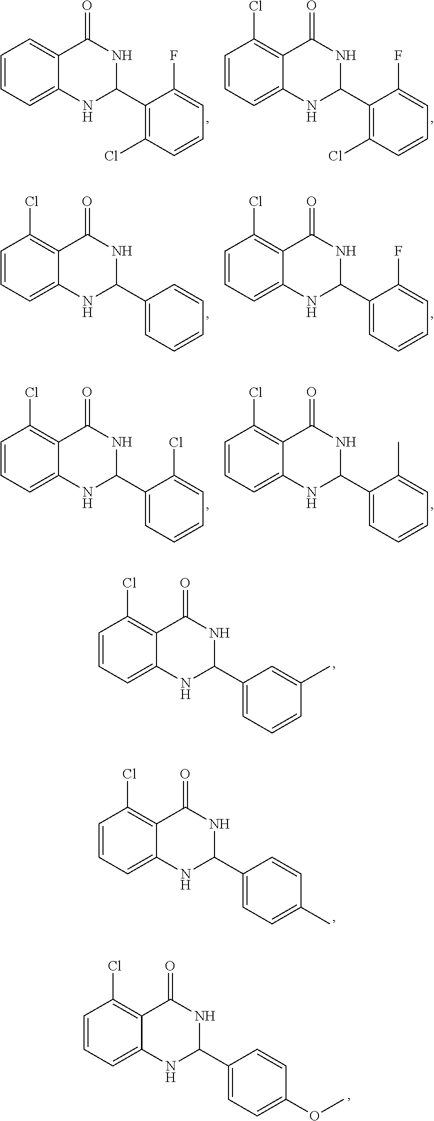

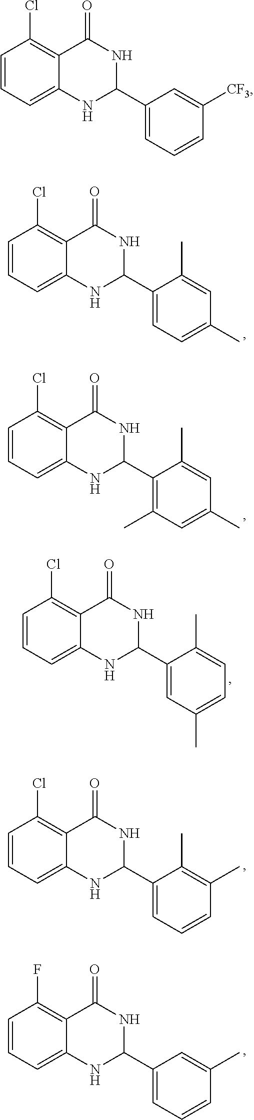

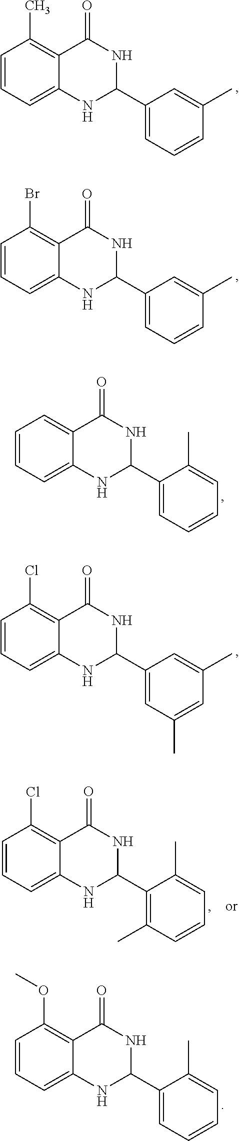 [SMILES: Cc1cccc(C2NC(=O)c3c(C)cccc3N2)c1 ; Cc1cccc(C2NC(=O)c3c(Br)cccc3N2)c1 ; Cc1ccccc1C1NC(=O)c2ccccc2N1 ; Cc1cc(C)cc(C2NC(=O)c3c(Cl)cccc3N2)c1 ; Cc1cccc(C)c1C1NC(=O)c2c(Cl)cccc2N1 ; COc1cccc2c1C(=O)NC(c1ccccc1C)N2]

In another aspect, the disclosure provides a compound having the formula I(b), wherein R² is pyridyl substituted with one methyl and R³ is chloro. In some embodiments, the methyl is a meta substituent.

In a further aspect, the disclosure provides a compound that is

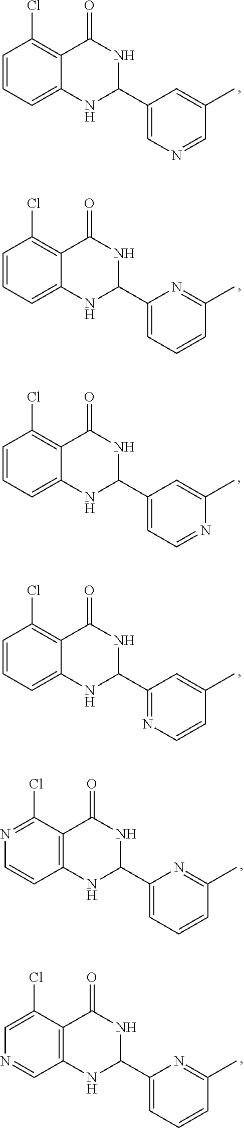 [SMILES: Cc1cncc(C2NC(=O)c3c(Cl)cccc3N2)c1 ; Cc1cccc(C2NC(=O)c3c(Cl)cccc3N2)n1 ; Cc1cc(C2NC(=O)c3c(Cl)cccc3N2)ccn1 ; Cc1ccnc(C2NC(=O)c3c(Cl)cccc3N2)c1 ; Cc1cccc(C2NC(=O)c3c(ccnc3Cl)N2)n1 ; Cc1cccc(C2NC(=O)c3c(Cl)cncc3N2)n1]

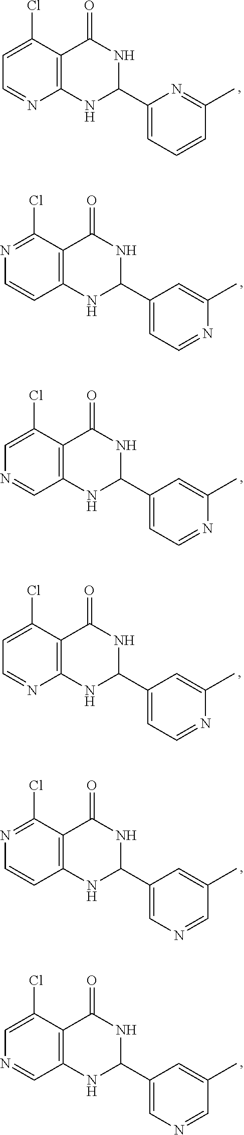 [SMILES: Cc1cccc(C2NC(=O)c3c(Cl)ccnc3N2)n1 ; Cc1cc(C2NC(=O)c3c(ccnc3Cl)N2)ccn1 ; Cc1cc(C2NC(=O)c3c(Cl)cncc3N2)ccn1 ; Cc1cc(C2NC(=O)c3c(Cl)ccnc3N2)ccn1 ; Cc1cncc(C2NC(=O)c3c(ccnc3Cl)N2)c1 ; Cc1cncc(C2NC(=O)c3c(Cl)cncc3N2)c1]

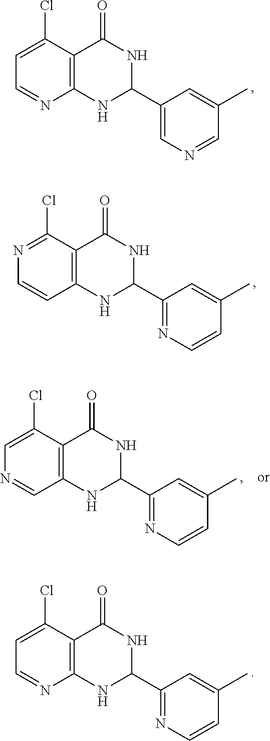 [SMILES: Cc1cncc(C2NC(=O)c3c(Cl)ccnc3N2)c1 ; Cc1ccnc(C2NC(=O)c3c(ccnc3Cl)N2)c1 ; Cc1ccnc(C2NC(=O)c3c(Cl)cncc3N2)c1 ; Cc1ccnc(C2NC(=O)c3c(Cl)ccnc3N2)c1]

In another aspect, the disclosure provides a compound of that is

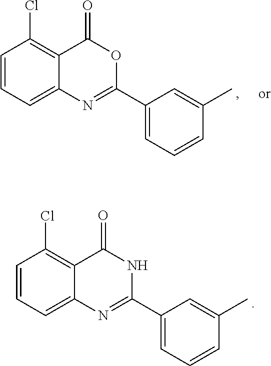 [SMILES: Cc1cccc(-c2nc3cccc(Cl)c3c(=O)o2)c1 ; Cc1cccc(-c2nc3cccc(Cl)c3c(=O)[nH]2)c1]

In another aspect the disclosure provides a compound of

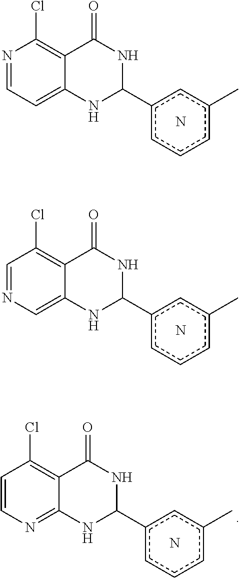

In one aspect, the disclosure provides a compound of formula II(a):

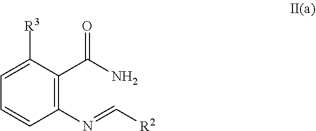

wherein R² is phenyl substituted with one or more halogen; and
R³ is hydrogen or halogen.

In another aspect, the disclosure provides a compound that is

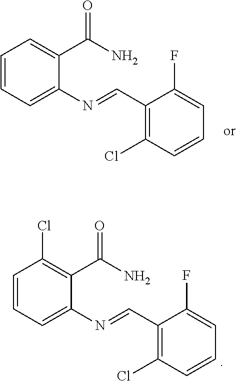 [SMILES: NC(=O)c1ccccc1/N=C/c1c(F)cccc1Cl ; NC(=O)c1c(Cl)cccc1/N=C/c1c(F)cccc1Cl]

In yet another aspect, the disclosure provides a compound of formula III(a):

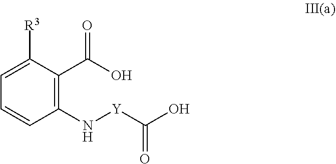

wherein Y is phenylene optionally substituted with halogen; and
R³ is hydrogen or halogen.

In a further aspect, the disclosure provides a compound that is

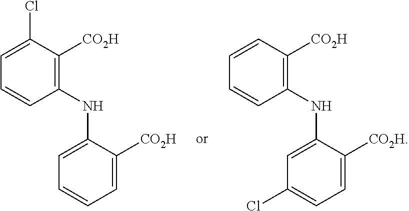 [SMILES: O=C(O)c1ccccc1Nc1cccc(Cl)c1C(=O)O ; O=C(O)c1ccccc1Nc1cc(Cl)ccc1C(=O)O]

In another aspect, the disclosure provides a compound of formula IV(a):

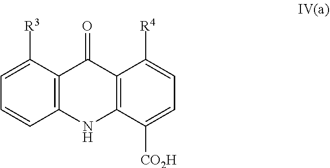

wherein R³ and R⁴ are independently selected from the group consisting of hydrogen and halogen.

In yet another aspect, the disclosure provides a compound of the formula IV(a), wherein R³ is chloro and R⁴ is hydrogen.

In another aspect, the disclosure provides a compound having an IC₅₀ value of from greater than 0 μM to 30 μM for inhibiting activity of polybromo-1 second bromodomain (PBRM1-BD2).

In yet another aspect, the disclosure provides a composition comprising the compound described herein and a pharmaceutically acceptable carrier. In some embodiments, the composition is formulated to be administered orally.

In another aspect, the disclosure provides a method of treating cancer in a subject in need thereof, wherein the method comprises administering an effective amount of the compound as described herein or the composition as described herein in order to treat the cancer. In some embodiments, the cancer is selected from renal cell carcinoma and prostate cancer. In some embodiments, the compound or the composition is administered in combination with cancer immunotherapy.

In a further aspect, the disclosure provides a method of inhibiting PBRM1-BD in a cell, the method comprising contacting the cell with an effective amount of one or more of the compounds as described herein. In some embodiments, the cell is in vivo in a subject in need thereof and the contacting comprises administering an effective amount of the one or more compounds. In some embodiments, the cell or subject is a cancer cell. In some embodiments, the cancer cell is a prostate cancer cell.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 shows selected pan-inhibitors of bromodomain family VIII. Atoms expected to interact with proteins through specific hydrogen or halogen bonds are highlighted in brown.

FIG. 2 demonstrates a summary of protein-detected NMR-based fragment screen targeting PBRM1-BD2. 1000 fragments were screened in pools of ≤12, and individual compound hits were identified through stepwise parsing of selected samples, yielding a final hit rate of 1.2% (left column). At each stage of parsing, identification of hit samples was aided by principal component analysis (middle column) and difference intensity analysis (right column) of the 2D HMQC spectra. Samples are colored according to k-means clustering of principle components. Throughout the screening process, samples selected as hits are represented as solid circles and bars, and samples containing compound 5 are marked with a star.

FIG. 3 (A) shows compounds synthesized to evaluate halogen bond formation between compound 7 and PBRM1-BD2 backbone. Box highlights the quinazolinone scaffold. IC₅₀ values were obtained using AlphaScreen assay. Tₘ shift values were obtained using DSF assay. Values shown are the average of three replicates and standard deviation. K_{d} value for each compound was obtained from ITC measurements.

FIG. 3 (B) shows overlaid ITC data for compounds 7 and 8.

FIG. 3 (C) demonstrates dose-response curve giving IC₅₀ values for 7 (0.2±0.02 μM) and 8 (6.3±1.4 μM), as tested by AlphaScreen assay.

FIG. 4 (A) shows heat maps showing the selectivity profile of selected compounds against the members of the bromodomain family. Inhibitors were screened at 10 μM concentration against 100 μM of selected bromodomains by DSF assay.

FIG. 4 (B) shows the s electivity profile of compounds 7 (blue, literature reported) and 16 (red, synthesized compound), as indicated on the phylogenetic tree of bromodomain family. Inhibitors were screened at 100 μM concentration against 10 μM of selected bromodomains by DSF assay.

FIG. 4 (C) shows an overlay of ITC binding curves of SMARCA4 bromodomain with compounds 7 (blue) and 16 (red).

FIG. 4 (D) shows ITC data for compound 16 with PBRM1-BD2.

FIG. 5 (A) shows cellular activity of PBRM1-BD2 inhibitors. Immunoblot of lysates from human cell lines with lentiviral expression of shRNA against PBRM1 or scrambled control shRNA. Cell lines include LNCaP (AR-positive prostate cancer cell line), PC3 (AR-negative prostate cancer cell line), RWPE-1 (prostate epithelial cell line), and HEK293T (transformed human embryonic kidney cell line).

FIG. 5 (B) shows cell viability after 6d incubation of cell lines expressing lentiviral shPBRM1 or shScrambled.

FIG. 5 (C) shows prostate cell line viability after 5d treatment with 10, 1, 0.1, or 0 μM of indicated compounds in 96 well plates. Viability is measured as luminescence units using CellTiter Glo®. n=3 for compound treatments, n=6 for DMSO alone.

FIG. 5 (D) shows dose curve measurement for compounds 16 and 34 in LNCaP cells. IC₅₀ was calculated from the variable slope dose curve generated using Prism9.

FIG. 5 (E) shows the viability of LNCaP cells expressing shRNA against PBRM1 (shPBRM1) or scrambled shRNA (shScram) after 5d treatment with 10, 1, 0.1, or 0 μM of compound 16. Viability is measured as luminescence units using CellTiter Glo®. N=9-12 ****=p<0.0001. Significance calculated using students t-test.

FIG. 5 (F) shows immunoblot analysis of streptavidin-mediated enrichment of biotinylated H3 or H3K14Ac/K18Ac/K23Ac/K27 Acpeptides added to LNCaP nuclear lysate containing 250 mM NaCl. Enrichment of PBRM1 and TATA-binding protein (TBP) was determined using immunoblot analysis.

FIG. 6 shows a summary of protein-detected NMR-based fragment screen of the Zenobia Library targeting PBRM1-BD2. 967 fragments were screened in pools of <12 and individual compound hits were identified through stepwise parsing of selected samples, yielding a final hit rate of 0.7% (left column). At each stage of parsing, identification of hit samples was aided by principal component analysis (middle column) and difference intensity analysis (right column) of the 2D HMQC spectra. Samples are colored according to k-means clustering of principle components. Throughout the screening process, samples selected as hits are represented as solid circles and bars.

FIG. 7 (A) shows ¹H, ¹⁵N SOFAST-HMQC overlays of PBRM1-BD2 titrated with increasing concentrations of 5 (structure in insert).

FIG. 7 (B) shows quantification of total chemical shift perturbations (¹H/¹⁵N Δδ chemical shift) manifested by 5 (6 mM) for individual amino acid residues of PBRM1-BD2.

FIG. 7 (C) shows mapping of substantially perturbed residues on the crystal structure of PBRM1-BD2 (PDB ID: 3LJW). The in silico docked pose of 5 into the active site of PBRM1-BD2 is included. Residues displaying CSPs>2s (red), between 1s and 2s (pink), or <1s (white) are indicated.

FIG. 7 (D) shows concentration-response curves of indicated residues used to calculate binding affinity (K_{d}=45.3±8.1 μM) of 5 for PBRM1-BD2 using GraphPad Prism.

FIG. 8 (A) shows ¹H, ¹⁵N SOFAST-HMQC overlays of PBRM1-BD2 titrated with increasing concentrations of 6 (structure in insert).

FIG. 8 (B) shows quantification of total chemical shift perturbations (¹H/¹⁵N Δδ chemical shift) manifested by 6 (1 mM) for individual amino acid residues of PBRM1-BD2.

FIG. 8 (C) shows mapping of substantially perturbed residues on the crystal structure of PBRM1-BD2 (PDB ID: 3LJW). The in silico docked pose of 6 into the active site of PBRM1-BD2 is included. Residues displaying CSPs>2s (red), between 1s and 2s (pink), or <1s (white) are indicated.

FIG. 8 (D) shows concentration-response curves of indicated residues used to calculate binding affinity (K_{d}=79±35 μM) of 6 for PBRM1-BD2 using GraphPad Prism.

FIG. 9 (A) shows ¹H, ¹⁵N SOFAST-HMQC overlays of PBRM1-BD2 titrated with increasing concentrations of 6a (structure in insert).

FIG. 9 (B) shows quantification of total chemical shift perturbations (¹H/¹⁵N Δδ chemical shift) manifested by 6a (1 mM) for individual amino acid residues of PBRM1-BD2.

FIG. 9 (C) shows mapping of substantially perturbed residues on the crystal structure of PBRM1-BD2 (PDB ID: 3LJW). The in silico docked pose of 6a into the active site of PBRM1-BD2 is included. Residues displaying CSPs>2s (red), between 1s and 2s (pink), or <1s (white) are indicated.

FIG. 9 (D) shows concentration-response curves of indicated residues used to calculate binding affinity (K_{d}=170±35 μM) of 6a for PBRM1-BD2 using GraphPad Prism.

FIG. 10 (A) shows ¹H, ¹⁵N SOFAST-HMQC overlays of PBRM1-BD2 titrated with increasing concentrations of 6b (structure in insert).

FIG. 10 (B) shows quantification of total chemical shift perturbations (¹H/¹⁵N Δδ chemical shift) manifested by 6b (6 mM) for individual amino acid residues of PBRM1-BD2.

FIG. 10 (C) shows mapping of substantially perturbed residues on the crystal structure of PBRM1-BD2 (PDB ID: 3LJW). The in silico docked pose of 6b into the active site of PBRM1-BD2 is included. Residues displaying CSPs>2s (red), between 1s and 2s (pink), or <1s (white) are indicated.

FIG. 10 (D) shows concentration-response curves of indicated residues used to calculate binding affinity (K_{d}=902±270 μM) of 6b for PBRM1-BD2 using GraphPad Prism.

FIG. 11 (A) shows ¹H, ¹⁵N SOFAST-HMQC overlays of PBRM1-BD2 titrated with increasing concentrations of 6c (structure in insert).

FIG. 11 (B) shows quantification of total chemical shift perturbations (¹H/¹⁵N Δδ chemical shift) manifested by 6c (1 mM) for individual amino acid residues of PBRM1-BD2.

FIG. 11 (C) shows mapping of substantially perturbed residues on the crystal structure of PBRM1-BD2 (PDB ID: 3LJW). The in silico docked pose of 6c into the active site of PBRM1-BD2 is included. Residues displaying CSPs>2s (red), between 1s and 2s (pink), or <1s (white) are indicated.

FIG. 11 (D) shows concentration-response curves of indicated residues used to calculate binding affinity (K_{d}=709±211 μM) of 6c for PBRM1-BD2 using GraphPad Prism.

FIG. 12 (A) shows ¹H, ¹⁵N SOFAST-HMQC overlays of PBRM1-BD2 titrated with increasing concentrations of 6d (structure in insert).

FIG. 12 (B) shows quantification of total chemical shift perturbations (¹H/¹⁵N Δδ chemical shift) manifested by 6d (6 mM) for individual amino acid residues of PBRM1-BD2.

FIG. 12 (C) shows mapping of substantially perturbed residues on the crystal structure of PBRM1-BD2 (PDB ID: 3LJW). The in silico docked pose of 6d into the active site of PBRM1-BD2 is included. Residues displaying CSPs>2s (red), between 1s and 2s (pink), or <1s (white) are indicated.

FIG. 12 (D) shows concentration-response curves of indicated residues used to calculate binding affinity (K_{d}=1387±262 μM) of 6d for PBRM1-BD2 using GraphPad Prism.

FIG. 13 (A) shows ¹H, ¹⁵N SOFAST-HMQC overlays of PBRM1-BD2 titrated with increasing concentrations of 6e (structure in insert).

FIG. 13 (B) shows quantification of total chemical shift perturbations (¹H/¹⁵N Δδ chemical shift) manifested by 6e (6 mM) for individual amino acid residues of PBRM1-BD2.

FIG. 13 (C) shows mapping of substantially perturbed residues on the crystal structure of PBRM1-BD2 (PDB ID: 3LJW). The in silico docked pose of 6e into the active site of PBRM1-BD2 is included. Residues displaying CSPs>2s (red), between 1s and 2s (pink), or <1s (white) are indicated.

FIG. 13 (D) shows concentration-response curves of indicated residues used to calculate binding affinity (K_{d}=1195±250 μM) of 6e for PBRM1-BD2 using GraphPad Prism.

FIG. 14 (A) shows ¹H, ¹⁵N SOFAST-HMQC overlays of PBRM1-BD2 titrated with increasing concentrations of 6g (structure in inert).

FIG. 14 (B) shows quantification of total chemical shift perturbations (¹H/¹⁵N Δδ chemical shift) manifested by 6g (6 mM) for individual amino acid residues of PBRM1-BD2.

FIG. 14 (C) shows mapping of substantially perturbed residues on the crystal structure of PBRM1-BD2 (PDB ID: 3LJW). The in silico docked pose of 6g into the active site of PBRM1-BD2 is included. Residues displaying CSPs>2s (red), between 1s and 2s (pink), or <1s (white) are indicated.

FIG. 14 (D) shows concentration-response curves of indicated residues used to calculate binding affinity (K_{d}=1210±267 μM) of 6g for PBRM1-BD2 using GraphPad Prism.

FIG. 15 (A) shows ¹H, ¹⁵N SOFAST-HMQC overlays of PBRM1-BD2 titrated with increasing concentrations of 6h (structure in insert).

FIG. 15 (B) shows quantification of total chemical shift perturbations (¹H/¹⁵N Δδ chemical shift) manifested by 6h (1 mM) for individual amino acid residues of PBRM1-BD2.

FIG. 15 (C) shows mapping of substantially perturbed residues on the crystal structure of PBRM1-BD2 (PDB ID: 3LJW). The in silico docked pose of 6h into the active site of PBRM1-BD2 is included. Residues displaying CSPs>2s (red), between 1s and 2s (pink), or <1s (white) are indicated.

FIG. 15 (D) shows concentration-response curves of indicated residues used to calculate binding affinity (K_{d}>2 mM) of 6h for PBRM1-BD2 using GraphPad Prism.

FIG. 16 (A) shows ¹H, ¹⁵N SOFAST-HMQC overlays of PBRM1-BD2 titrated with increasing concentrations of 6i (structure in insert).

FIG. 16 (B) shows quantification of total chemical shift perturbations (¹H/¹⁵N Δδ chemical shift) manifested by 6i (1 mM) for individual amino acid residues of PBRM1-BD2.

FIG. 16 (C) shows mapping of substantially perturbed residues on the crystal structure of PBRM1-BD2 (PDB ID: 3LJW). The in silico docked pose of 6i into the active site of PBRM1-BD2 is included. Residues displaying CSPs>2s (red), between 1s and 2s (pink), or <1s (white) are indicated.

FIG. 16 (D) shows concentration-response curves of indicated residues used to calculate binding affinity (K_{d}>2 mM) of 6i for PBRM1-BD2 using GraphPad Prism.

FIG. 17 (A) shows ¹H, ¹⁵N SOFAST-HMQC overlays of PBRM1-BD2 titrated with increasing concentrations of 6j (structure in insert).

FIG. 17 (B) shows quantification of total chemical shift perturbations (¹H/¹⁵N Δδ chemical shift) manifested by 6j (1 mM) for individual amino acid residues of PBRM1-BD2.

FIG. 17 (C) shows mapping of substantially perturbed residues on the crystal structure of PBRM1-BD2 (PDB ID: 3LJW). The in silico docked pose of 6j into the active site of PBRM1-BD2 is included. Residues displaying CSPs>2s (red), between 1s and 2s (pink), or <1s (white) are indicated.

FIG. 17 (D) shows concentration-response curves of indicated residues used to calculate binding affinity (K_{d}>2 mM) of 6j for PBRM1-BD2 using GraphPad Prism.

FIG. 18 shows IC₅₀ curves from AlphaScreen assays using 0.2 μM His₆-tagged PBRM1-BD2 and 0.1 μM biotinylated H3K14ac peptide.

FIG. 19 (A) shows cellular activity of BD2 ligands. (A) Prostate cell line viability after 5d treatment with 10, 1, 0.1 or 0 μM of indicated compounds in 96 well plates. Viability is measured as luminescence units using CellTiter Glo®. N=3 for compound treatments, n=6 for DMSO alone.

FIG. 19 (B) shows dose curve measurement for compound 25 treatment in LNCaP, PC3 and HEK293T cells. IC₅₀ calculated from variable slope dose curve generated using Prism9.

FIG. 19 (C) shows viability of LNCaP and PC3 prostate cancer cell lines treated with indicated concentrations of compounds 7 and 16.

FIG. 19 (D) shows the viability of LNCaP cells expressing shRNA against PBRM1 (shPBRM1) or scrambled shRNA (shScram) after 5d treatment with 10, 1, 0.1 or 0 μM of compound 16. Viability is measured as luminescence units using CellTiter Glo®. N=9-12 ****=p<0.0001. Significance calculated using students t-test.

FIG. 19 (E) shows immunoblot analysis after streptavidin-mediated peptide pull-downs with biotinylated H3 or H3K14Ac peptide from LNCaP nuclear lysate containing 150 mM NaCl. Enrichment of PBRM1 and TATA binding protein (TBP) were determined by immunoblot analysis.

The drawings illustrate only example embodiments and are therefore not to be considered limiting of the scope of the embodiments described herein, as other embodiments are within the scope of the disclosure.

### DETAILED DESCRIPTION OF THE INVENTION

In the present invention, the inventors provide novel chemical probes targeting PBRM1 bromodomains, specifically PBRM1 bromodomain 2, to better understand the association between aberrant PBRM1 chromatin binding and cancer pathogenesis. Mutations in PBRM1 cluster to the bromodomains are found in many human cancers and the PBRM1 acetyl-lysine binding activity is important in maintaining normal gene transcription. Via a fragment-based, protein-detected NMR approach, compounds were screened against the second bromodomain of PBRM1. The inventors discover that compounds of formula (I), specifically formula I(a), I(b), II(a), III(a), and IV(a) are found to be nanomolar potent, cell-active inhibitors displaying exclusive selectivity for binding to PBRM1 over SMARCA2/4 and other bromodomain-containing proteins. The identified ligand inhibits the association of full length PBRM1/PBAF to acetylated histone peptides in cell lysates, and selectively inhibits the growth of PBRM1-dependent prostate cancer cell lines.

### Novel PBRM1-BD2 Inhibitors

In some aspects of the current disclosure, novel inhibitors of the PBRM1 second domain (PBRM1-BD2) are provided. In some embodiments, the PBRM1-BD2 inhibitors have a formula (I):

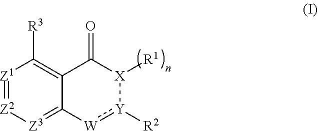

wherein X is selected from C, N, NH, or 0;
Y is selected from C or phenylene optionally substituted with halogen;
W is N or NH;
Z¹, Z², and Z³ are independently selected from CH and N;
n is 0 or 1;
R¹ is hydrogen, and R² is selected from the group consisting of carboxyl, pyridyl optionally substituted with one or more alkyl, and phenyl optionally substituted with one or more substituents selected from the group consisting of halogen, alkyl, alkoxy, and haloalkyl, or R¹ and R² together form a phenyl optionally substituted with one or more substituents selected from the group consisting of carboxyl and halogen; and
R³ is selected from the group consisting of hydrogen, alkoxy, alkyl, and halogen.

In one embodiment, the compound has a formula I(a):

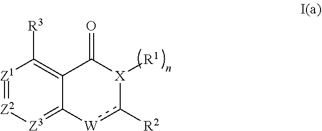

wherein X is N or O;
W is N or NH;
Z¹, Z², and Z³ are independently selected from CH and N;
n is 0 or 1;
R¹ is hydrogen; and
R² is selected from pyridyl optionally substituted with one or more alkyl, and phenyl optionally substituted with one or more substituents selected from the group consisting of halogen, alkyl, alkoxy, and haloalkyl.

The inventor discovered that compounds having a 2-phenylhydroquinazolinone scaffold, a 2-pyridylquinazolinone scaffold, or a 2-pyridyl pyridopyrimidinone scaffold provide potential PBRM1-BD2 inhibitors of suitable binding affinity (K_{d}) and inhibitory activity (IC₅₀). As such, in some embodiments, the compound has a formula I(b):

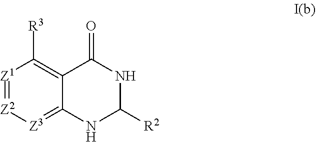

wherein Z¹, Z², and Z³ are independently selected from CH and N;
R² is selected from the group consisting of pyridyl optionally substituted with one or more alkyl and phenyl optionally substituted with one or more substituents selected from the group consisting of halogen, alkyl, alkoxy, and haloalkyl; and
R³ is selected from the group consisting of hydrogen, alkoxy, alkyl, and halogen.

In some embodiments, the novel PBRM1-BD2 inhibitors of suitable binding affinity (K_{d}) and inhibitory activity (IC₅₀) has a 2-phenylhydroquinazolinone scaffold. In some embodiments, R² in the compound of formula I(b) as described herein is phenyl optionally substituted with one or more substituents selected from the group consisting of fluoro, chloro, methyl, methoxy, and trifluoromethyl, R³ in the compound of formula I(b) as described herein is hydrogen, fluoro, chloro, bromo, or methyl, and Z¹, Z², and Z³ are CH. In some embodiments, such compound is selected from.

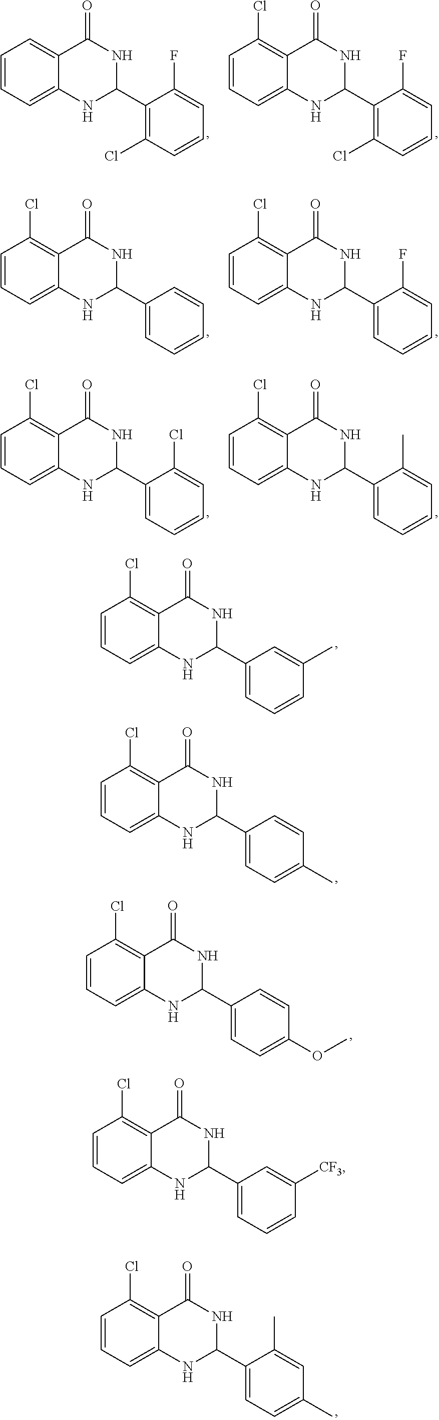

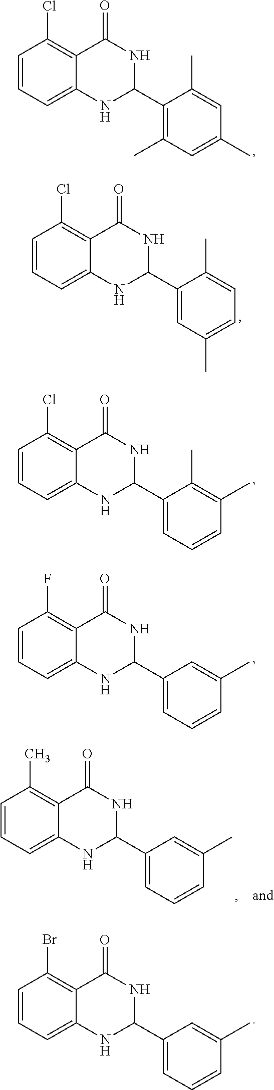

In some embodiments, the novel PBRM1-BD2 inhibitors of suitable binding affinity (K_{d}) and inhibitory activity (IC₅₀) has a 2-pyridylhydroquinazolinone scaffold or a 2-pyridyl pyridopyrimidinone scaffold. In some embodiments, R² in the compound of formula I(b) as described herein is pyridyl substituted with one methyl and R³ is chloro. In some embodiments, such compound is selected from:

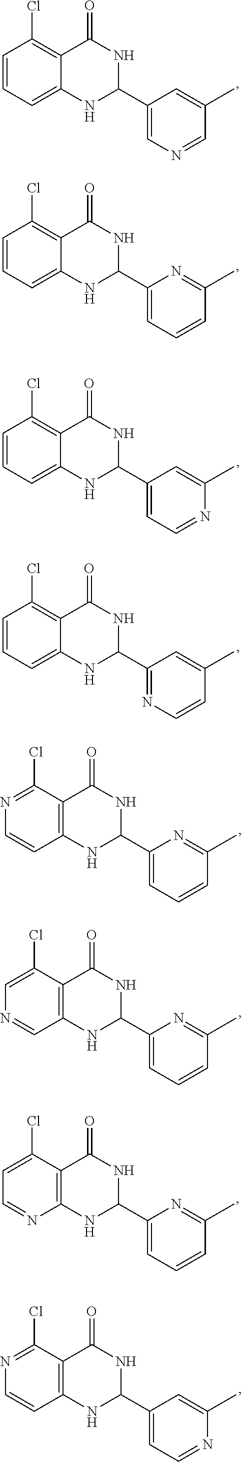

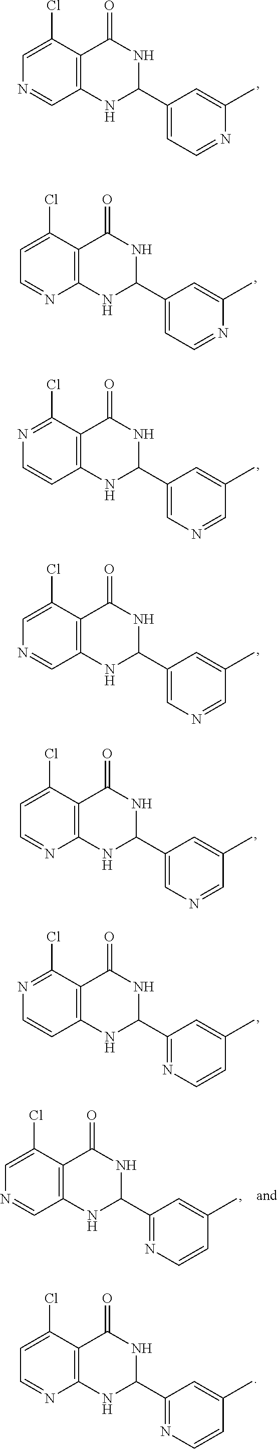

In some embodiments, the compound of formula I(b) as described herein is:

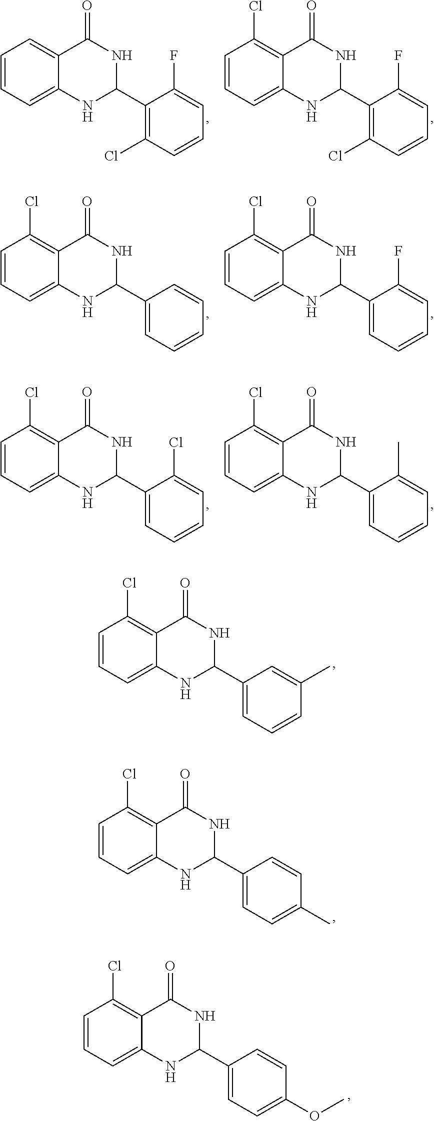

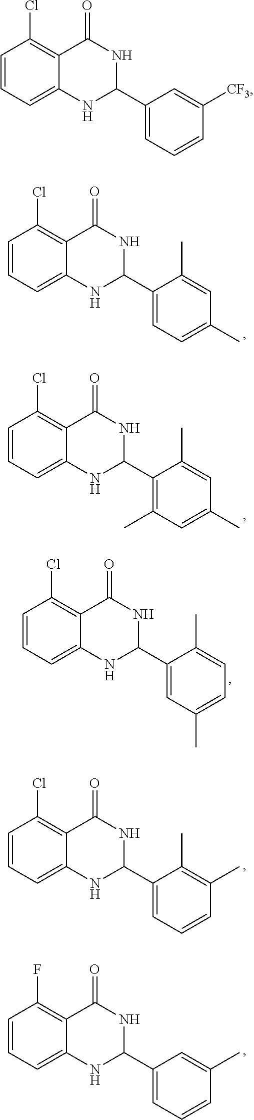

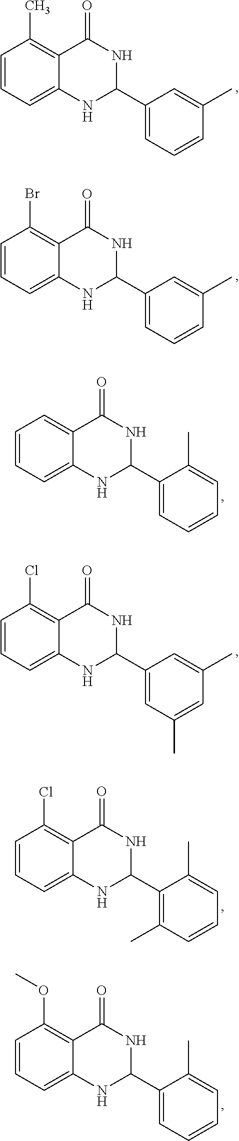 [SMILES: Cc1cccc(C2NC(=O)c3c(C)cccc3N2)c1 ; Cc1cccc(C2NC(=O)c3c(Br)cccc3N2)c1 ; Cc1ccccc1C1NC(=O)c2ccccc2N1 ; Cc1cc(C)cc(C2NC(=O)c3c(Cl)cccc3N2)c1 ; Cc1cccc(C)c1C1NC(=O)c2c(Cl)cccc2N1 ; COc1cccc2c1C(=O)NC(c1ccccc1C)N2]

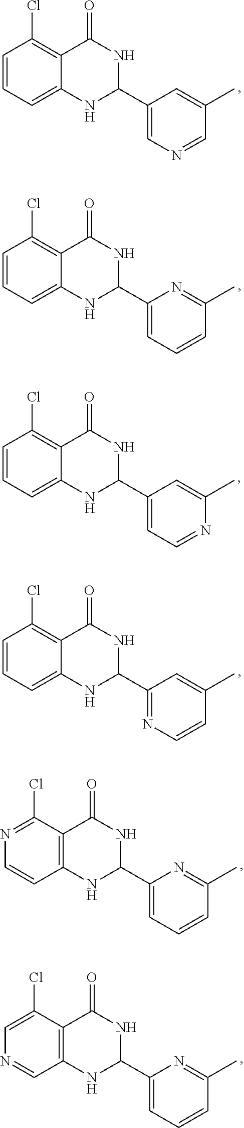 [SMILES: Cc1cncc(C2NC(=O)c3c(Cl)cccc3N2)c1 ; Cc1cccc(C2NC(=O)c3c(Cl)cccc3N2)n1 ; Cc1cc(C2NC(=O)c3c(Cl)cccc3N2)ccn1 ; Cc1ccnc(C2NC(=O)c3c(Cl)cccc3N2)c1 ; Cc1cccc(C2NC(=O)c3c(ccnc3Cl)N2)n1 ; Cc1cccc(C2NC(=O)c3c(Cl)cncc3N2)n1]

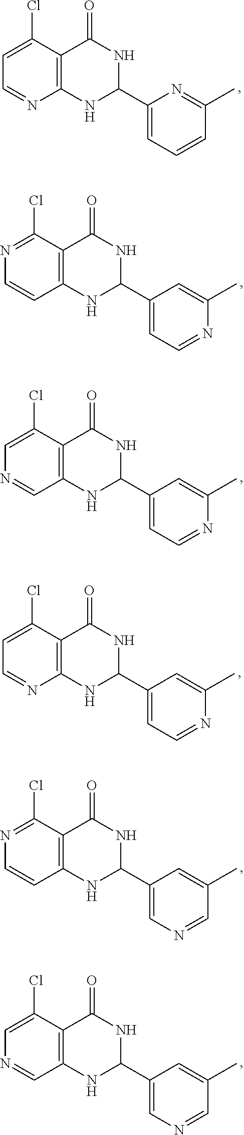 [SMILES: Cc1cccc(C2NC(=O)c3c(Cl)ccnc3N2)n1 ; Cc1cc(C2NC(=O)c3c(ccnc3Cl)N2)ccn1 ; Cc1cc(C2NC(=O)c3c(Cl)cncc3N2)ccn1 ; Cc1cc(C2NC(=O)c3c(Cl)ccnc3N2)ccn1 ; Cc1cncc(C2NC(=O)c3c(ccnc3Cl)N2)c1 ; Cc1cncc(C2NC(=O)c3c(Cl)cncc3N2)c1]

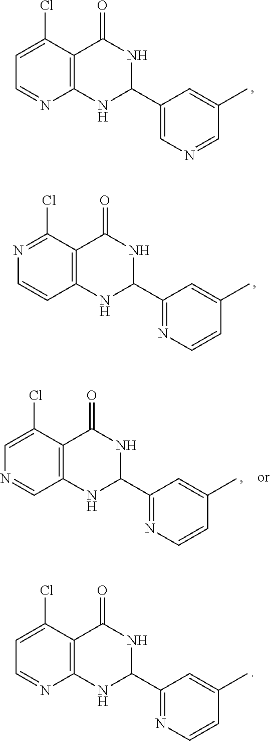 [SMILES: Cc1cncc(C2NC(=O)c3c(Cl)ccnc3N2)c1 ; Cc1ccnc(C2NC(=O)c3c(ccnc3Cl)N2)c1 ; Cc1ccnc(C2NC(=O)c3c(Cl)cncc3N2)c1 ; Cc1ccnc(C2NC(=O)c3c(Cl)ccnc3N2)c1]

In some embodiments, the compound of formula I(a) is selected from:

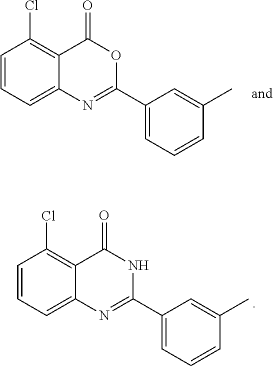 [SMILES: Cc1cccc(-c2nc3cccc(Cl)c3c(=O)o2)c1 ; Cc1cccc(-c2nc3cccc(Cl)c3c(=O)[nH]2)c1]

In some embodiments, the compound as described herein has a formula II(a):

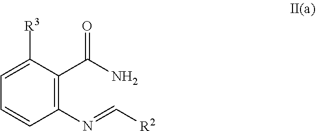

wherein R² is phenyl substituted with one or more halogen; and
R³ is hydrogen or halogen.

In some embodiments, the compound of formula II(a) is selected from

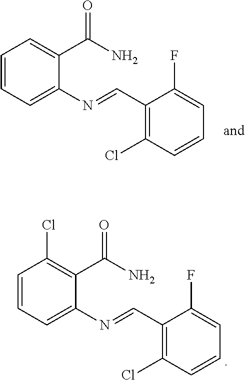 [SMILES: NC(=O)c1ccccc1/N=C/c1c(F)cccc1Cl ; NC(=O)c1c(Cl)cccc1/N=C/c1c(F)cccc1Cl]

In some embodiments, the compound as described herein has a formula III(a):

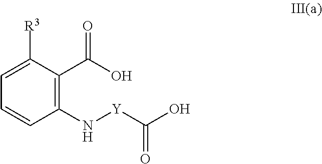

wherein Y is phenylene optionally substituted with halogen; and
R³ is hydrogen or halogen.

In some embodiments, the compound of formula III(a) is selected from:

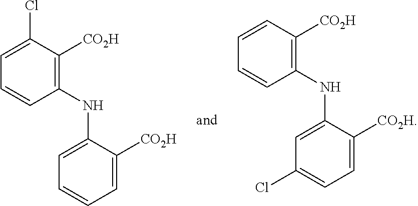 [SMILES: O=C(O)c1ccccc1Nc1cccc(Cl)c1C(=O)O ; O=C(O)c1ccccc1Nc1cc(Cl)ccc1C(=O)O]

In some embodiments, the compound as described herein has a formula

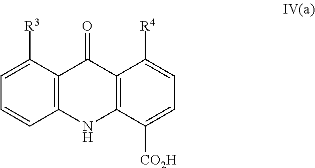

wherein R³ and R⁴ are independently selected from the group consisting of hydrogen and halogen.

In some embodiments, R³ is chloro and R⁴ is hydrogen in the compound of IV(a) as described herein.

In some embodiments, the compound as described herein has an IC₅₀ value of from greater than 0 μM to 30 μM for inhibiting activity of polybromo-1 second bromodomain (PBRM1-BD2). In some embodiments, the compound as described herein has an IC₅₀ value of from greater than 0 μM to 28 μM, from greater than 0 μM to 25 μM, from greater than 0 μM to 22 μM, from greater than 0 μM to 20 μM, from greater than 0 μM to 17 μM, from greater than 0 μM to 15 μM, from greater than 0 μM to 12 μM, from greater than 0 μM to 10 μM, from greater than 0 μM to 7 μM, from greater than 0 μM to 5 μM, from greater than 0 μM to 3 μM, from greater than 0 μM to 1 μM, from greater than 0 μM to 0.5 μM, for inhibiting activity of polybromo-1 second bromodomain (PBRM1-BD2).

In some embodiments, the compound is

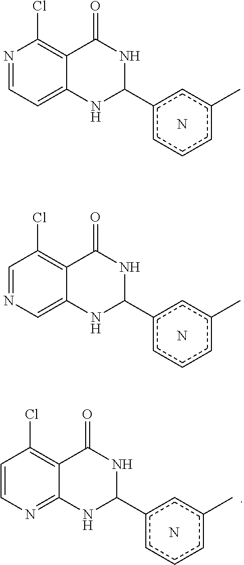

### Chemical Entities

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

The term “carboxy” or “carboxyl” as used herein refers to the radical —COOH or its corresponding salts, e.g. —COONa, etc.

The term “phenyl” refers to a mono-substituted benzene ring and has a formula of —C₆H₅—. The term “phenylene” refers to a di-substituted benzene ring and has a formula of —C₆H₄—.

The term “halogen” refers to halogen atoms F, Cl, Br, and I, or halogen substituents fluoro (—F), chloro (—Cl), bromo (—Br), and iodo- (—I).

The term “haloalkyl” is art-recognized and refers to an alkyl group, as defined above, having halogen atoms, as defined above, replacing one or more hydrogen atoms. Representative haloalkyl groups include trifluoromethyl, dibromoethyl, monochloropropyl, and the like.

The term “pyridyl” refers to a group derived from pyridine by removal of a hydrogen atom from a ring carbon atom in pyridine. The pyridyl group has a formula —C₅H₄N.

As used herein, the dashed line ( [symbol] ) in the formulae as described herein is an optional single bond. The symbol having one dashed line on top of a solid line (“ [symbol] ”) refers to a single bond that may optionally be a double bond.

A person of ordinary skill in the art would be able to choose the substituents that fulfill the valency rules. For example, in a non-solvated or non-salt form of a compound, nitrogen typically has three bonds attached to it and oxygen typically has two bonds attached to it.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### Pharmaceutical Compositions

In some embodiments, pharmaceutical compositions are provided herein. In some embodiments, the pharmaceutical compositions comprise a compound with a formula (I), I(a), I(b), II(a), III(a), and/or IV(a) as described herein. In some embodiments, the pharmaceutical compositions further comprise a pharmaceutically acceptable carrier.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a PBRM1-BD2 inhibitor may be administered as a single compound or in combination with another PBRM1-BD2 inhibitor.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder, e.g., a cell proliferative disorder such as cancer. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder, e.g., a cell proliferative disease or disorder including cancer.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

The term “pharmaceutically acceptable” means approved by a regulatory agency of the Federal or a state government or listed in the U.S. Pharmaceutically acceptable” carriers are known in the art and include, but are not limited to, suitable diluents, preservatives, solubilizers, emulsifiers, liposomes, nanoparticles and adjuvants. For example, pharmaceutically acceptable carriers may comprise 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solutions include propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

The term “carrier” refers to a diluent, adjuvant, excipient, or vehicle with which the pharmaceutical composition (e.g., immunogenic or vaccine formulation) is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should be selected according to the mode of administration. The vaccine compositions may include a pharmaceutical carrier, excipient, or diluent, which are nontoxic to the cell or subject being exposed thereto at the dosages and concentrations employed. Often a pharmaceutical diluent is in an aqueous pH buffered solution. Examples of pharmaceutical carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN brand surfactant, polyethylene glycol (PEG), and PLURONICS™ surfactant.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods of Treatment

In one embodiment, the invention provides a method of treating cancer in a subject comprising administering to the subject a therapeutically effective amount of a composition comprising at least one compound described herein. In use, the compounds of the present invention are more cytotoxic to cancer cells than to non-cancerous cells.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

The Inventors demonstrated that the disclosed compounds effectively inhibit the proliferation of cancer cells, specifically, for example, prostate cancer. Therefore, in one aspect of the current disclosure, methods of treating cancer are provided. In some embodiments, methods of treating cancer in a subject in need thereof comprise administering a therapeutically effective amount of a compound described herein, wherein the compound comprises a formula selected from formula (I):

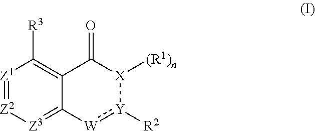

wherein X is selected from C, N, NH, and O;
   Y is selected from C and phenylene optionally substituted with halogen;
   W is N or NH;
   Z¹, Z², and Z³ are independently selected from CH and N;
   n is 0 or 1;
   R¹ is hydrogen, and R² is selected from the group consisting of carboxyl, pyridyl optionally substituted with one or more alkyl, and phenyl optionally substituted with one or more substituents selected from the group consisting of halogen, alkyl, alkoxy, and haloalkyl, or R¹ and R² together form a phenyl optionally substituted with one or more substituents selected from the group consisting of carboxyl and halogen; and
   R³ is selected from the group consisting of hydrogen, alkoxy, alkyl, and halogen.

In some embodiments, methods of treating cancer in a subject in need thereof comprise administering a therapeutically effective amount of a compound described herein, wherein the compound comprises a formula selected from formula I(a):

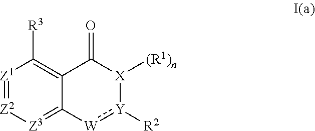

wherein X is N or O;
   W is N or NH;
   Z¹, Z², and Z³ are independently selected from CH and N;
   n is 0 or 1;
   R¹ is hydrogen;
   R² is selected from pyridyl optionally substituted with one or more alkyl, and phenyl optionally substituted with one or more substituents selected from the group consisting of halogen, alkyl, alkoxy, and haloalkyl; and
   R³ is selected from the group consisting of hydrogen, alkoxy, alkyl, and halogen.

In some embodiments, methods of treating cancer in a subject in need thereof comprise administering a therapeutically effective amount of a compound described herein, wherein the compound comprises a formula selected from formula I(b):

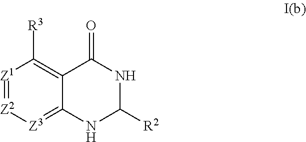

wherein Z¹, Z², and Z³ are independently selected from CH and N;
R² is selected from the group consisting of pyridyl optionally substituted with one or more alkyl and phenyl optionally substituted with one or more substituents selected from the group consisting of halogen, alkyl, alkoxy, and haloalkyl; and
R³ is selected from the group consisting of hydrogen, alkoxy, alkyl, and halogen

In some embodiments, methods of treating cancer in a subject in need thereof comprise administering a therapeutically effective amount of a compound described herein, wherein the compound comprises a formula selected from formula II(a):

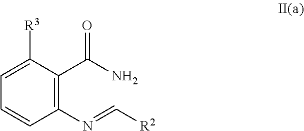

wherein R² is phenyl substituted with one or more halogen; and
R³ is hydrogen or halogen.

In some embodiments, methods of treating cancer in a subject in need thereof comprise administering a therapeutically effective amount of a compound described herein, wherein the compound comprises a formula selected from formula III(a):

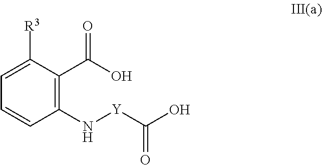

wherein Y is phenylene optionally substituted with halogen; and
R³ is hydrogen or halogen.

In some embodiments, methods of treating cancer in a subject in need thereof comprise administering a therapeutically effective amount of a compound described herein, wherein the compound comprises a formula selected from formula IV(a):

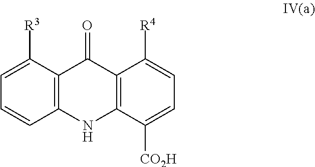

wherein R³ and R⁴ are independently selected from the group consisting of hydrogen and halogen.

In some embodiments, methods of treating cancer in a subject in need thereof comprise administering a therapeutically effective amount of a compound described herein, wherein the compound comprises a formula selected from the group consisting of formula I, formula I(b), formula II(a), formula III(a), formula IV(a), as described herein, and any combinations thereof.

The inventors unexpectedly discovered a structure-activity relationship between the structure of the potential PBRM1-BD2 inhibitor and its binding affinity and inhibiting potency towards PBRM1-BD2. For compounds of formula I(b) containing a dihydroquinazolinone scaffold, the binding affinity/potency towards PBRM1-BD2 could be improved by the introduction of a chlorine at the C-5 position of the quinazolinone scaffold. Moreover, the modifications on the phenyl ring substituted at the 2-position of the dihydroquinazolinone scaffold are also promising. Specifically, a methyl at the meta- and/or ortho-position of the phenyl in the 2-phenyl substituted dihydroquinazolinone structure showed increased inhibitory activities relative to the 2-phenyl substituted dihydroquinazolinone structure with no substitution on the phenyl group. In some embodiments, the dihydropyrimidinone ring is also found to be critical for the inhibitory activity of compounds towards PBRM1-BD2. In some embodiments, compounds containing an imino-benzamide scaffold (formula II(a)), a 2-(phenylamino)benzoic acid scaffold (formula III(a)), or a tricyclic compound containing a 9-oxo-9,10-dihydroacridine-4-carboxylic acid scaffold (formula (IV(a)) also provide inhibiting potency comparable to compounds of formula I(b) as described herein.

The compounds and composition comprising the compounds described herein can be further used in methods with a combination to other cancer therapies. For example, suitable other cancer therapies may depend on the type of cancer, such as, but not limited to, chemotherapies (e.g. tyrosine kinase inhibitors), immunotherapies, checkpoint inhibitors, hormone therapies (e.g., androgen deprivation therapy in prostate cancer), anti-angiogenic agents, and standard of care cancer therapies. Suitable checkpoint inhibitors include, but are not limited to, for example, agents capable of blockade of T cell immune checkpoint receptors, including but not limited to PD-1, PD-L1, TIM-3, LAG-3, CTLA-4, and CSF-1R and combinations of such checkpoint inhibitors. In some embodiments, the immune checkpoint inhibitors include anti-PD-1 antibody, anti-PD-L1 antibody, anti-CTLA4 antibody, anti-LAG-3 antibody, and/or anti-TIM-3 antibody. Suitable inhibitors include, for example, tremelimumab, atezolizumab, ipilumumab, pembrolizumab, and nivolumab, tislelizumab, among others. The inhibitor need not be an antibody, but can be a small molecule or other polymer. In a preferred embodiment, the checkpoint inhibitor is a PD-1 inhibitor, PD-L1 inhibitor, CTLA-4 inhibitor, or the like.

PD-1 or PD-L1 inhibitors may include, but are not limited to, antibodies, peptides, small molecules, antisense RNAs, cDNAs, miRNAs, siRNAs, aptamers, oligonucleotides, and the like. Examples include, but are not limited to, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83,545,509, 8,900,587 and EP2170959; atezolizumab is an anti-PD-L1 available from Genentech, Inc. (Roche) and described in U.S. Pat. No. 8,217,149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi), spartalizumab (PDR001, Novartis), camrelizumav (AiRuiKa, Hengrui Medicine Co.), sintillimab (Tyvyt, Innovent Biologics/Eli Lilly), KN035 (Envafolimab, Tracon Pharmaceuticals); tislelizumab available from BeiGene and described in U.S. Pat. No. 8,735,553; among others and the like. Other PD-1 and PD-L1 that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others. The term “checkpoint inhibitor therapy” refers to the form of cancer immunotherapy that block inhibitory checkpoints and thereby restore immune system function. Such therapies are known by those skilled in the art. In some embodiments, the PD-1 inhibitor is selected from the group consisting of Nivolumab (anti-PD-1), Pembrolizumab (anti-PD-1), and combinations thereof. In some embodiments, the PD-L1 inhibitor is selected from atezolizumab, avelumab, and durvalumab, among others.

By “subject” we mean mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. The term “subject” does not denote a particular age or sex. In a particular embodiment, the subject is a human. By “cancer’ or “tumor” we mean any abnormal proliferation or uncontrolled growth of cells, including solid and non-solid tumors. The methods of the present invention can be used to treat any cancer, any metastases thereof, and any chemo-residual growth thereof, including, but not limited to, carcinoma, lymphoma, blastoma, sarcoma, and leukemia. Suitable cancers able to be treated by the compounds and compositions, methods and kits described herein include, but are not limited to, prostate cancer, lymphoma, breast cancer, colon cancer, small-cell lung cancer, non-small cell lung cancer, ovarian cancer, cervical cancer, gastrointestinal cancer, pancreatic cancer, liver cancer, bladder cancer, hepatoma, colorectal cancer, uterine cancer, endometrial carcinoma, salivary gland carcinoma, mesothelioma, kidney cancer (e.g. clear cell renal cell carcinomia), vulval cancer, thyroid cancer, hepatic carcinoma, skin cancer (e.g. melanoma and squamous cell cancer), brain cancer (e.g. neuroblastoma and glioblastoma), myeloma, various types of head and neck cancer, acute lymphoblastic leukemia, acute myeloid leukemia, Ewing sarcoma, and peripheral neuroepithelioma. The composition and methods of the present disclosure can also be utilized to treat non-solid tumor cancers such as non-Hodgkin's lymphoma, leukemia and the like. In a particular embodiment, the cancer is prostate cancer.

The term “metastasis,” “metastatic tumor” or “secondary tumor” refers to cancer cells that have spread to a secondary site, e.g., outside of the primary tumor tissue. Secondary sites include, but are not limited to, the lymphatic system, skin, distant organs (e.g., liver, stomach, pancreas, brain, etc.) and the like.

The present disclosure also provides methods of reducing or inhibiting cancer cell growth in a subject having cancer, the method comprising administering an effective amount of the compounds or composition described herein to reduce or inhibit cancer cell growth.

For purposes of the present invention, “treating” or “treatment” describes the management and care of a subject for combating the disease, condition, or disorder. Treating includes the administration of the compound or composition described herein to reduce, prevent, ameliorate and/or improve the onset of the symptoms or complications, alleviating the symptoms or complications, or reducing or eliminating the disease, condition, or disorder.

For example, treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation.

By “administering” we mean any means for introducing the compounds of the present invention into the body, preferably into the systemic circulation or intratumoral delivery. Examples include but are not limited to oral, buccal, sublingual, pulmonary, transdermal, transmucosal, as well as subcutaneous, intraperitoneal, intravenous, and intramuscular injection.

By “therapeutically effective amount” we mean an amount effective, at dosages and for periods of time necessary, to achieve the desired therapeutic result, such as reduction or reversal of angiogenesis in the case of cancers, or reduction or inhibition of cancer growth. A therapeutically effective amount of the compounds of the invention may vary according to factors such as the disease state, age, sex, and weight of the subject, and the ability of the compounds to elicit a desired response in the subject. Dosage regimens may be adjusted to provide the optimum therapeutic response. A therapeutically effective amount is also one in which any toxic or detrimental effects of the compounds of the present invention are outweighed by the therapeutically beneficial effects.

Kits. In another embodiment, the present invention provides a kit comprising a pharmaceutical composition comprising the compounds of the present invention and instructional material. By “instructional material” we mean a publication, a recording, a diagram, or any other medium of expression which is used to communicate the usefulness of the pharmaceutical composition of the invention for one of the purposes set forth herein in a human. The instructional material can also, for example, describe an appropriate dose of the pharmaceutical composition of the invention. The instructional material of the kit of the invention can, for example, be affixed to a container which contains a pharmaceutical composition of the invention or be shipped together with a container which contains the pharmaceutical composition. Alternatively, the instructional material can be shipped separately from the container with the intention that the instructional material and the pharmaceutical composition be used cooperatively by the recipient.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

The following examples are shown to further illustrate the disclosed invention and are not intended to limit the disclosed invention, as recited in the claims.

### Example 1: Development of Cell-Active, Selective, and Potent Inhibitors for Polybromo-1 Using Protein-Detected NMR-Based Fragment Screen and Structure-Activity Relationship Studies

Bromodomains are ˜110 amino acid protein modules that bind acetylated lysine residues on histones and other nuclear proteins to epigenetically regulate gene expression. Polybromo-1 (PBRM1) contains a unique six bromodomains and is a critical chromatin-targeting subunit of the polybromo-associated BRG1- or BRM-associated factors (PBAF) chromatin remodeling complex. Mutations in PBRM1 cluster to the bromodomains and are found in many human cancers, demonstrating the importance of PBRM1 acetyl-lysine binding activity in maintaining normal gene transcription. As a result, selective chemical probes targeting PBRM1 bromodomains are required to better understand the association between aberrant PBRM1 chromatin binding and cancer pathogenesis. Previous chemical probes targeting the fifth bromodomain of PBRM1 also bind the structurally similar bromodomains of SMARCA2 and SMARCA4 with nanomolar potency. In this study, we used a fragment-based, protein-detected NMR approach to screen ˜2000 compounds against the second bromodomain of PBRM1. NMR titration studies identified 12 hits with binding affinities ranging from 50-2000 μM. Inhibitor binding was further validated using isothermal titration calorimetry (ITC), differential scanning fluorimetry (DSF), and AlphaScreen as secondary biophysical assays. Structure-activity relationship (SAR) studies of the tightest-binding fragment hit resulted in nanomolar potent, cell-active inhibitors displaying exclusive selectivity for binding to PBRM1 over SMARCA2/4 and other bromodomain-containing proteins. The best-identified ligand inhibits the association of full length PBRM1/PBAF to acetylated histone peptides in cell lysates, and selective inhibits the growth of PBRM1-dependent prostate cancer cell lines.

### INTRODUCTION

Post-translational modification of histones and other nuclear proteins epigenetically regulate gene expression without altering the underlying genomic sequence. Lysine acetylation is a particularly abundant epigenetic modification with widespread influence over transcription.¹ Bromodomains specifically bind acetylated lysine residues on histone tails and other nuclear proteins to facilitate acetylation-dependent protein-protein interactions. The human genome encodes 61 bromodomains distributed across 46 different proteins.² Based on similarities in sequence and structure, bromodomains are further divided into eight subfamilies.² Polybromo-1 (PBRM1) is a member of family VIII.², 3 PBRM1 uniquely contains six consecutive bromodomains along with two bromo-adjacent homolog domains (BAH), and a high mobility group (HMG).², 3 PBRM1 is a subunit of polybromo-associated BRG1- or BRM-associated factors (PBAF) complex, a member of ATP-dependent SWItch/Sucrose Non-Fermenting (SWI/SNF) chromatin remodeling complexes.³

Bromodomains are implicated in the pathogenesis of various disease states, including cancer,⁴ inflammation,^{4, 5} autoimmune,^{4, 5} and neurological disorders.⁶ Canonically, PBRM1 has been characterized as a tumor suppressor protein and gene regulator via the chromatin-targeting function of PBAF.⁷ To this end, mutations in the Pbrm1 gene are frequently observed in cancer, specifically in clear cell renal cell carcinoma (ccRCC),⁸ suggesting a tumor-suppressive role. However, patients with metastatic ccRCC and loss of Pbrm1 gene function responded better to anti-PD1 cancer immunotherapy compared to patients with Pbrm1 gene function intact.⁹ Also, the expression of T-cell cytotoxicity genes was inversely correlated with PBRM1 expression in many human cancers.⁹ Treatment-resistant tumor cells in mice became responsive to immunotherapy when the Pbrm1 gene was inactivated.¹⁰ Mice inoculated with PBRM11 deficient B16F10 tumor cells exhibited delayed tumor growth and extended survival rate after treatment with anti-PD1 cancer immunotherapy.¹⁰ In contrast, the normal expression and activity of PBRM1 are critical for the progression of certain prostate cancer subtypes, ¹¹⁻¹³ suggesting context-dependent function. The conflicting studies regarding the function of PBRM1 in cancer emphasize the need for selective PBRM1 inhibitors for use either as chemical probes or further development into clinical candidates. We envision PBRM1 inhibitors may be useful clinically for certain prostate cancer subtypes or when used in combination with cancer immunotherapies.

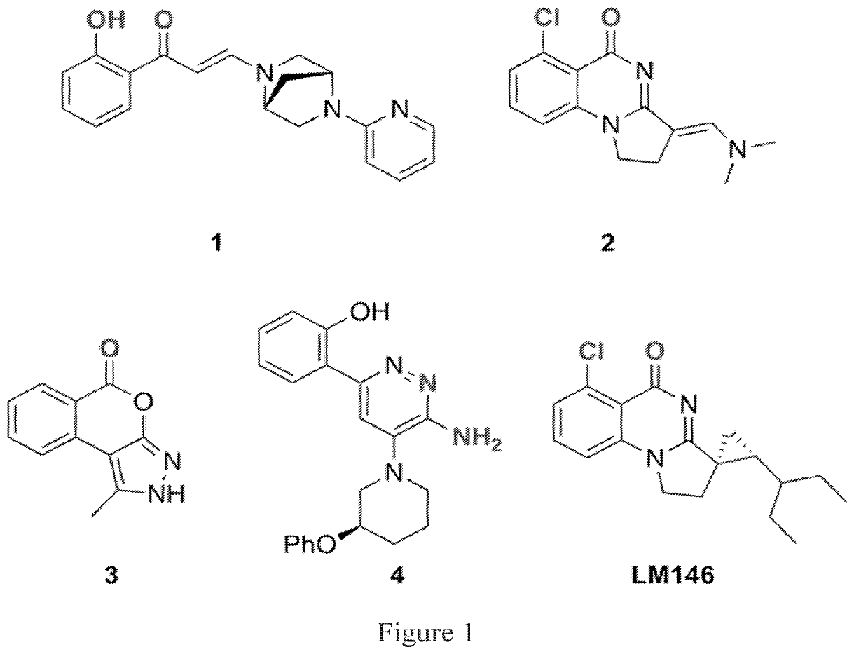

A few pan-inhibitors/chemical probes of the bromodomain family VIII have been reported. The first chemical probe reported is PFI-3 (1, FIG. 1),^{14, 15} developed from a salicylic acid fragment. The probe showed excellent selectivity for family VIII bromodomains and unusual binding mode by displacing structural water molecules, otherwise conserved in other bromodomains. Other inhibitors targeting the fifth bromodomain of PBRM1 (PBRM1-BD5) include quinazolinones (2),^{16, 17} isochromanones (3),¹⁸ and 6-aminopyrazines (4) (FIG. 1);¹⁹ however, none of these previously reported compounds are selective towards PBRM1 bromodomains, especially within family VIII bromodomains. PFI-3 (1),^{14, 15} fused quinazolines (2),¹⁷ and 6-aminopyrazines (4)¹⁹ exhibited nanomolar binding potencies against PBRM1-BD5, SMARCA2, and SMARCA4. Fused isochromanones (3)¹⁸ exhibited moderate binding affinities towards PBRM1-BD5 and had limited stability and solubility. The development of fused quinazolines into LM146¹⁶ selective to PBRM1 was recently published; however, LM146 still binds to SMARCA4 with micromolar affinity. Thus, there is an unmet need for a chemical probe that is selective for PBRM1 bromodomains to investigate the biological function of PBRM1 and as a starting point for clinical development.

Previous studies demonstrated that targeting PBRM1-BD5 results in off-target effects of also binding to bromodomains of SMARCA2 and SMARCA4;^{14, 15, 17, 19} PBRM1-BD1 does not have the conserved Asn residue in its binding pocket and therefore not critical for chromatin binding;^{2, 20} PBRM1-BD3 and PBRM1-BD6 are predicted not to bind to acetylated lysine residues on histones.⁷ As our previous studies have shown that the function of PBRM1 as a growth and gene expression regulator is dependent on the second bromodomain mediating binding of PBAF to chromatin,⁷ we targeted PBRM1-BD2 using a protein-detected NMR-based fragment screen with the overall goal of selective inhibition of PBRM1 function. The fragment screen identified several unique scaffolds otherwise not reported to bind bromodomains. We also report detailed structure-activity-relationship (SAR) studies of the tightest binding fragment hit, resulting in a series of nanomolar-potent, selective, and cell-active inhibitors of PBRM1. Partial SAR studies on the second-tightest binding hit are also reported. The compounds exhibit excellent selectivity for PBRM1-BD2 compared to other bromodomains within the family VIII and amongst the broader bromodomain superfamily.

### Results and Discussion

Protein-Detected NMR Fragment Screening of the Second Bromodomain of PBRM1

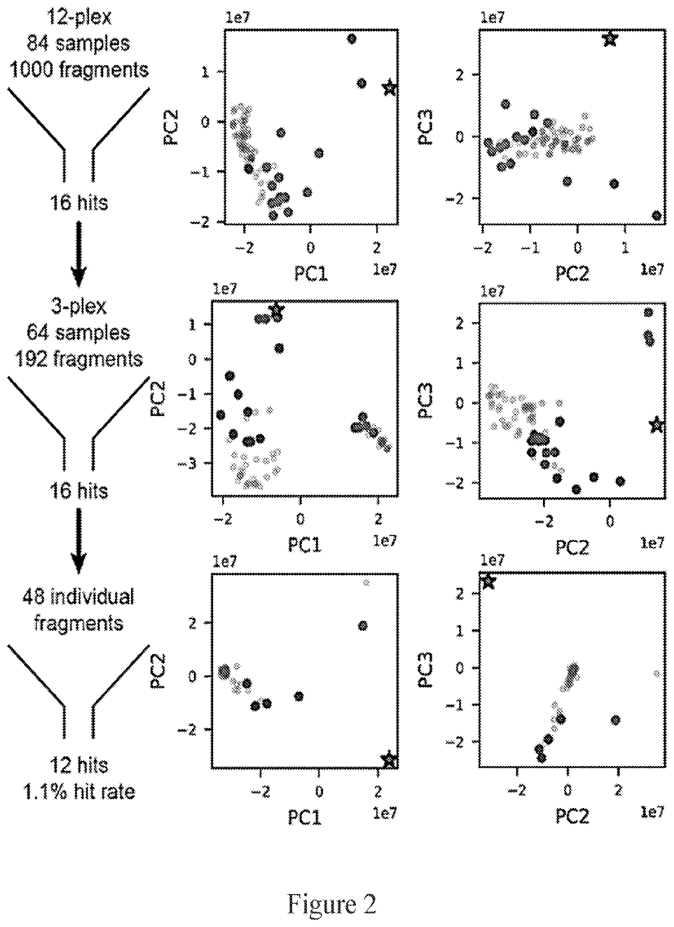

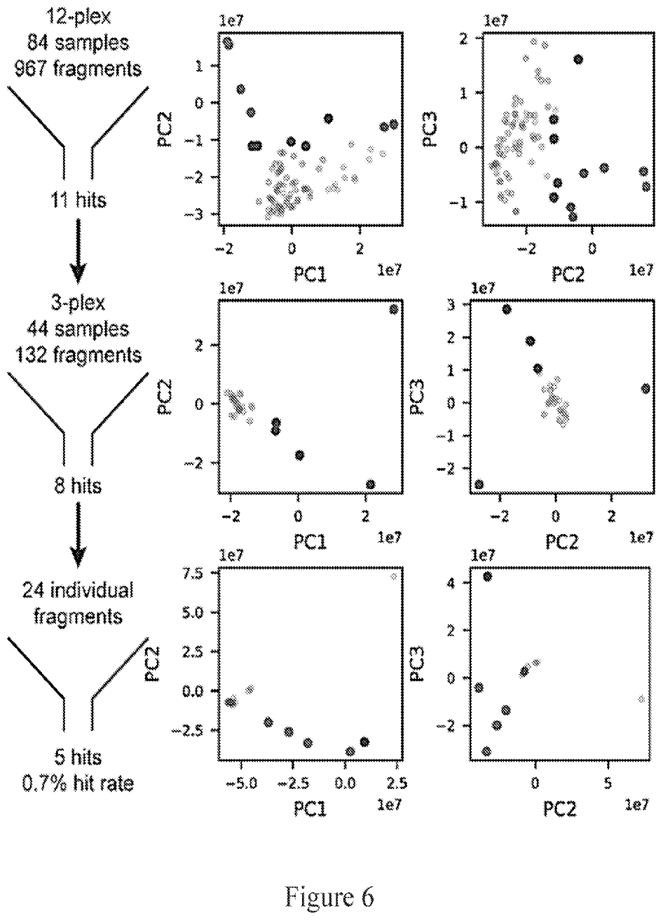

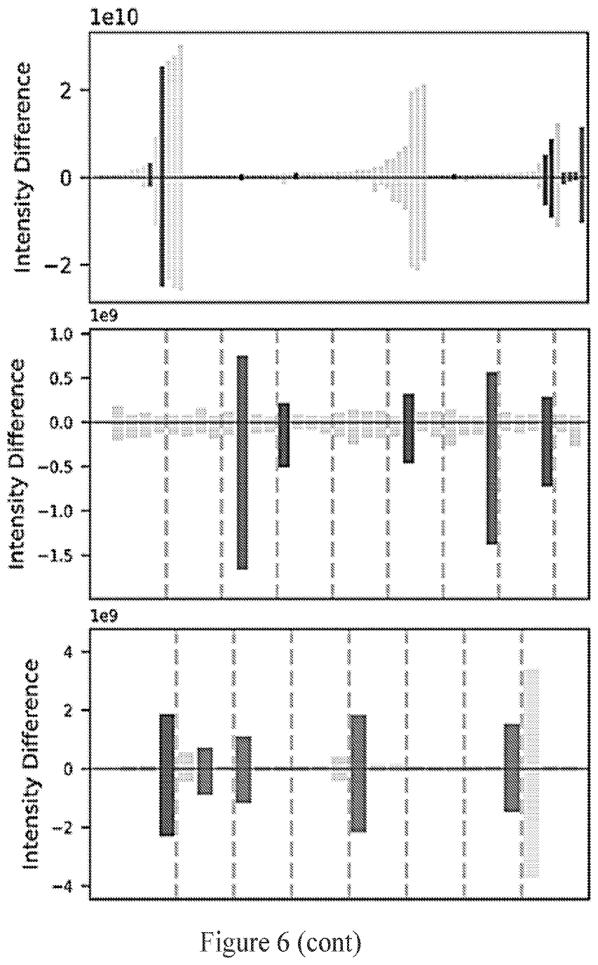

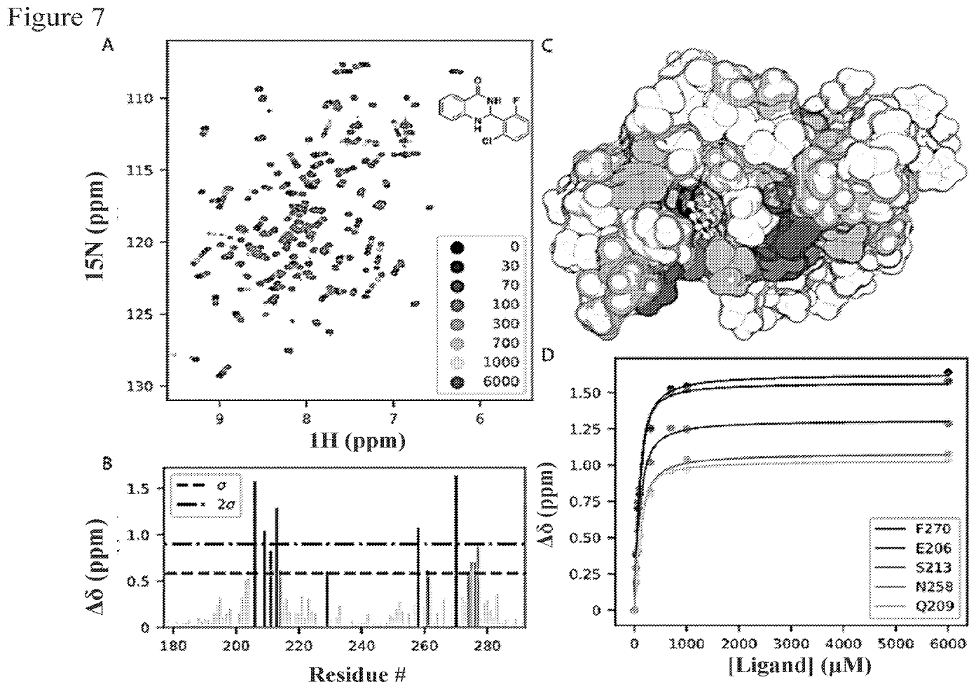

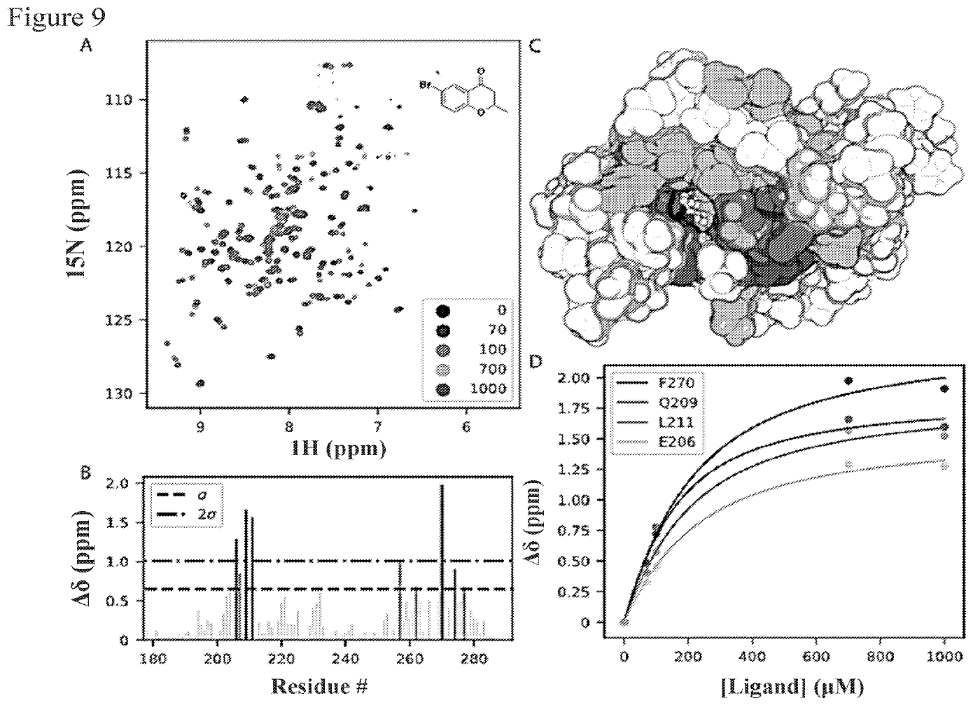

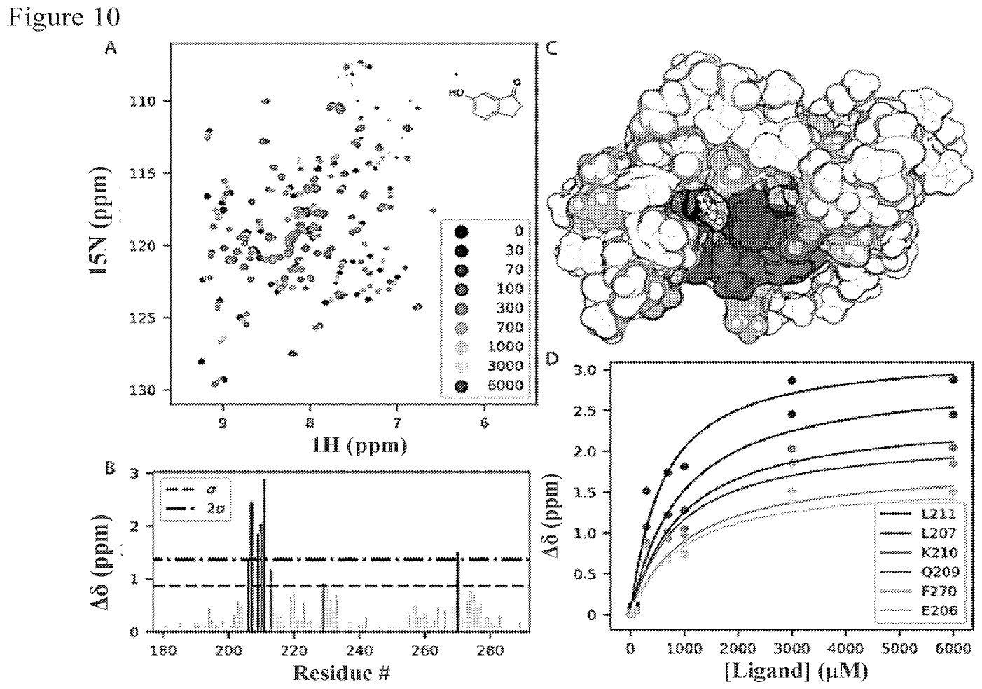

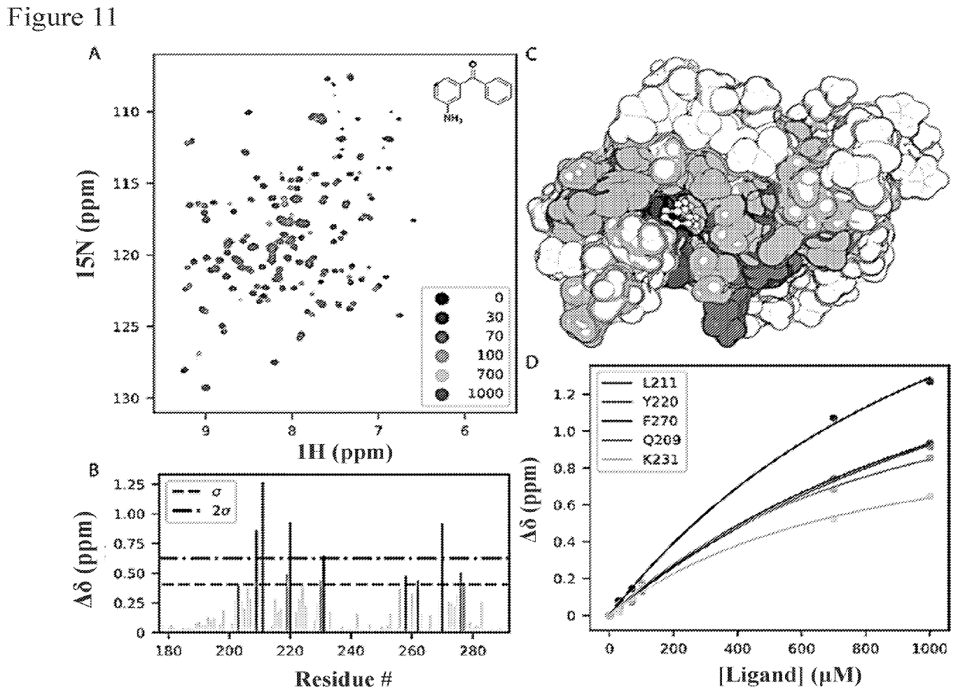

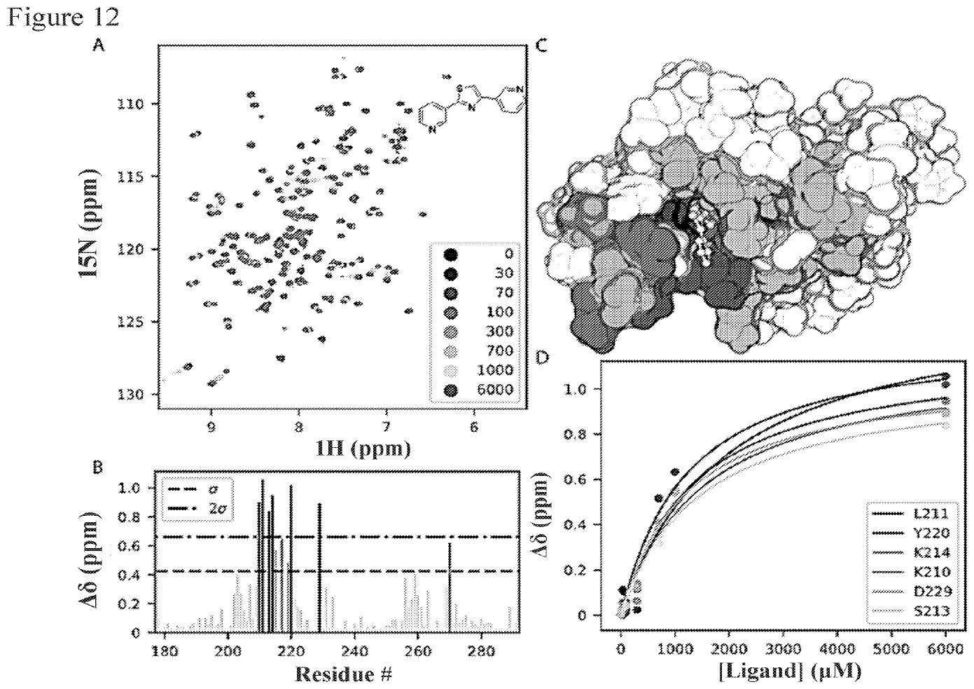

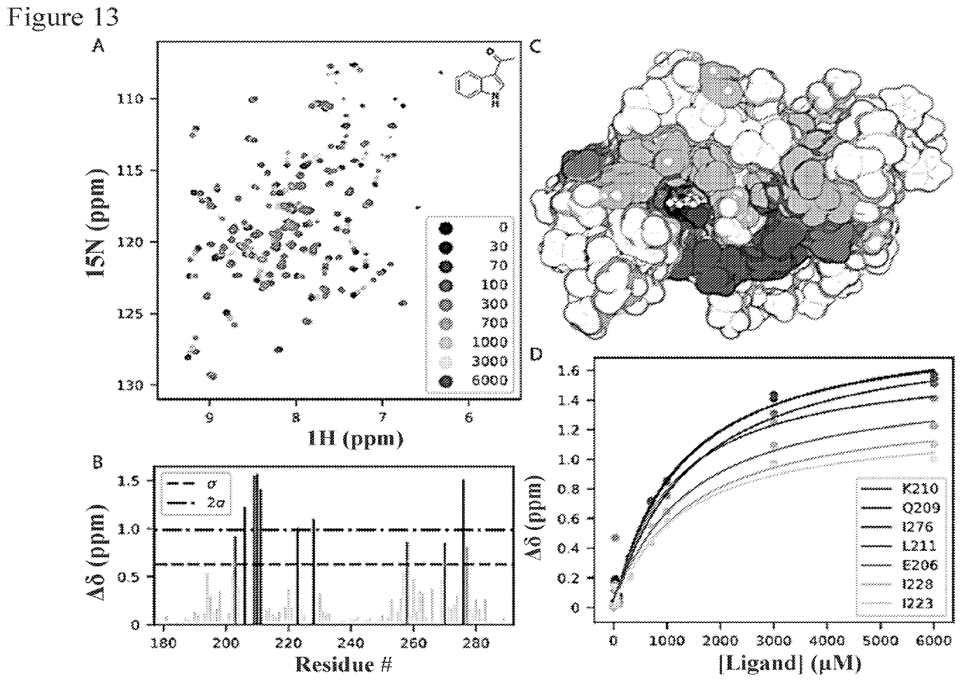

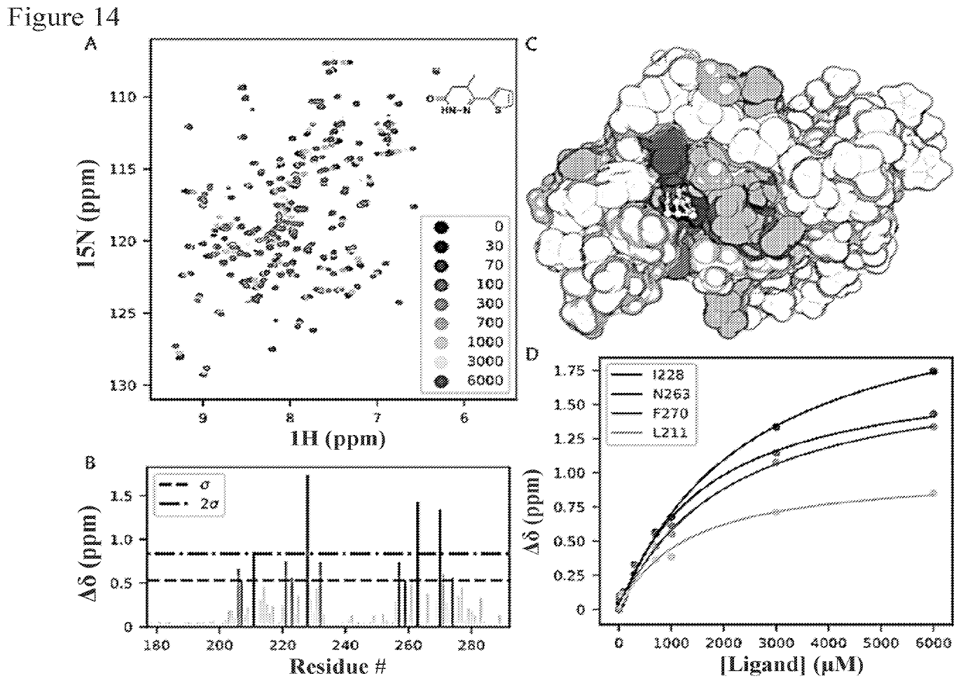

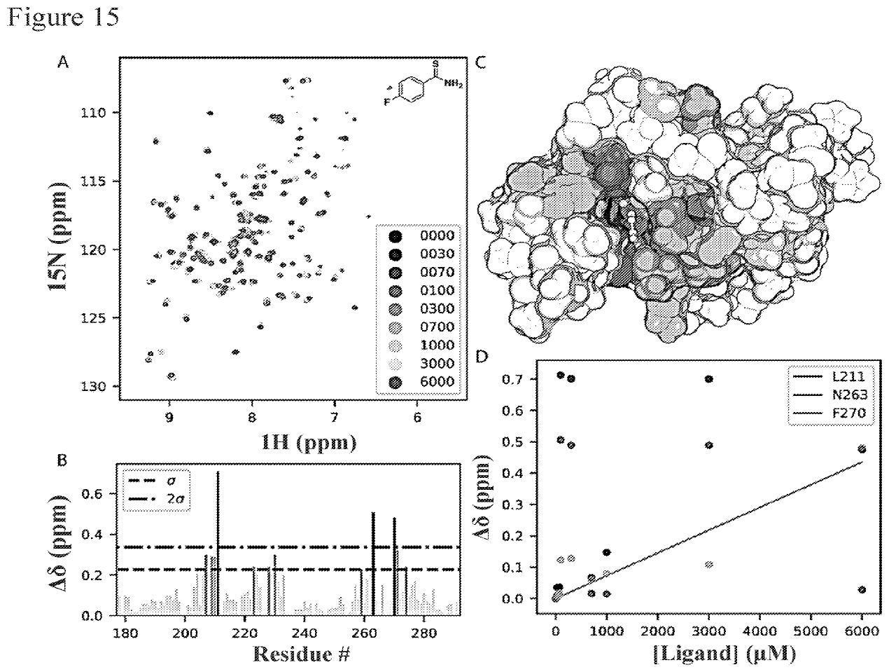

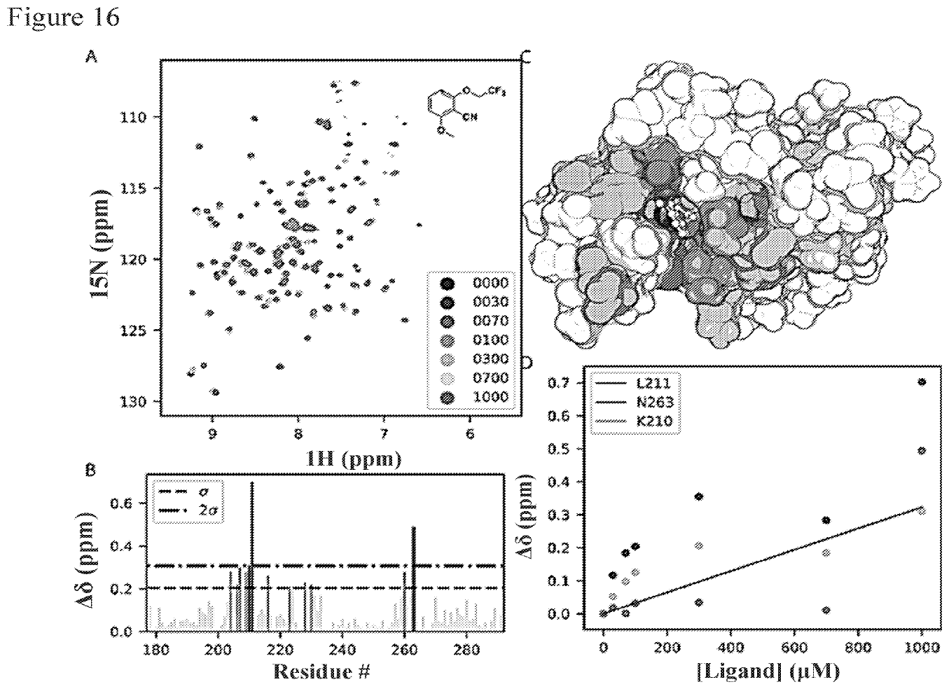

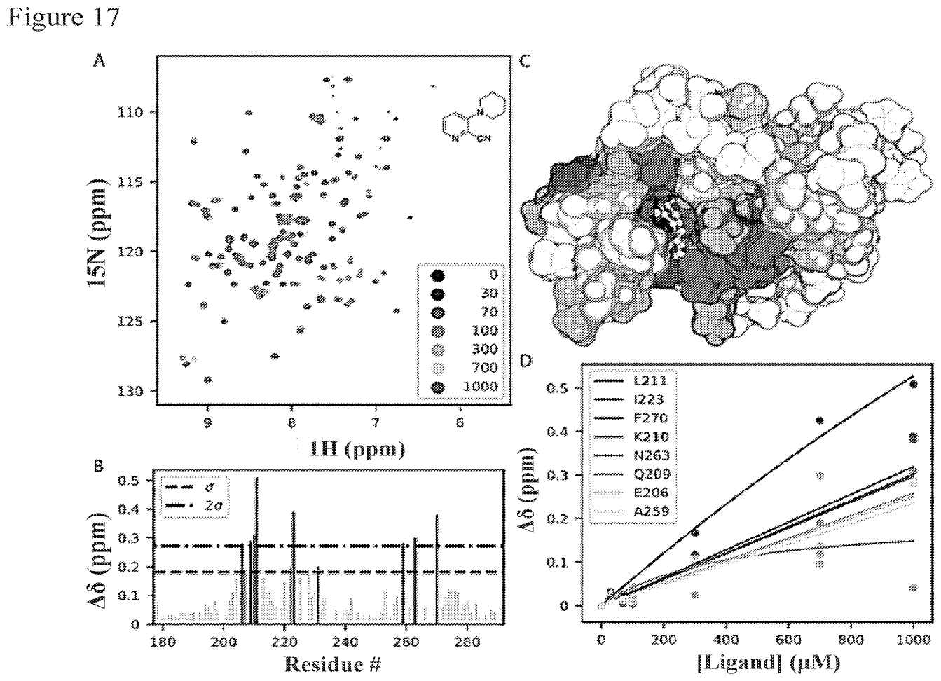

Protein-detected NMR-based fragment screening has the advantage of providing information about both binding affinity and the amino acid residues involved in binding. Our recent success with a protein-detected NMR-based, fragment-screening approach with the second bromodomain of BRD4²¹ prompted us to adopt the same biophysical technique to develop inhibitors of the second bromodomain of PBRM1 (PBRM1-BD2). Commercial libraries from Maybridge and Zenobia consisting of 1968 total fragments, all following the ‘rule of three’²² and an average molecular weight of 154 Da ranging from 94-286 Da, were screened against PBRM1-BD2 using protein-detected NMR. ¹H-¹⁵N SOFAST-HMQC NMR spectra were recorded with uniformly ¹⁵N-labeled PBRM1-BD2 in the absence/presence of mixtures of 12 fragments. In each screening step, ‘hit’ samples were identified using a combination of difference intensity analysis (DIA), principal component analysis (PCA), and manual inspection of spectral overlays as we recently described.²¹ The hit 12-fragment (12-plex) mixture samples were then parsed into four pools of 3 compounds (3-plex), followed by individual fragments from the 3-plex mixture hits to identify the fragment responsible for the observed chemical shift perturbations in the HMQC spectra. The screen identified twenty-seven 12-plexs as hits which gave twenty-four 3-plexs as hits which subsequently yielded seventeen individual fragments as hits (FIGS. 2 & 6), with a hit rate of 1.2% (Maybridge, FIG. 2) and 0.7% (Zenobia, FIG. 6). Some of the hits (12-plexs or 3-plexs) did not result in an individual fragment as hit. The fragment hits manifested varied K_{d} values ranging from 50-2000 μM, calculated by plotting chemical shift perturbations of amino acid residues of PBRM1-BD2 against increasing concentrations of the fragment (Table 1, S1, FIG. 7-17). Varied scaffolds were identified as hits, including quinazolinones, acridones, chromanones, triazoles, indenones, indoles, thiazoles, and thiols.

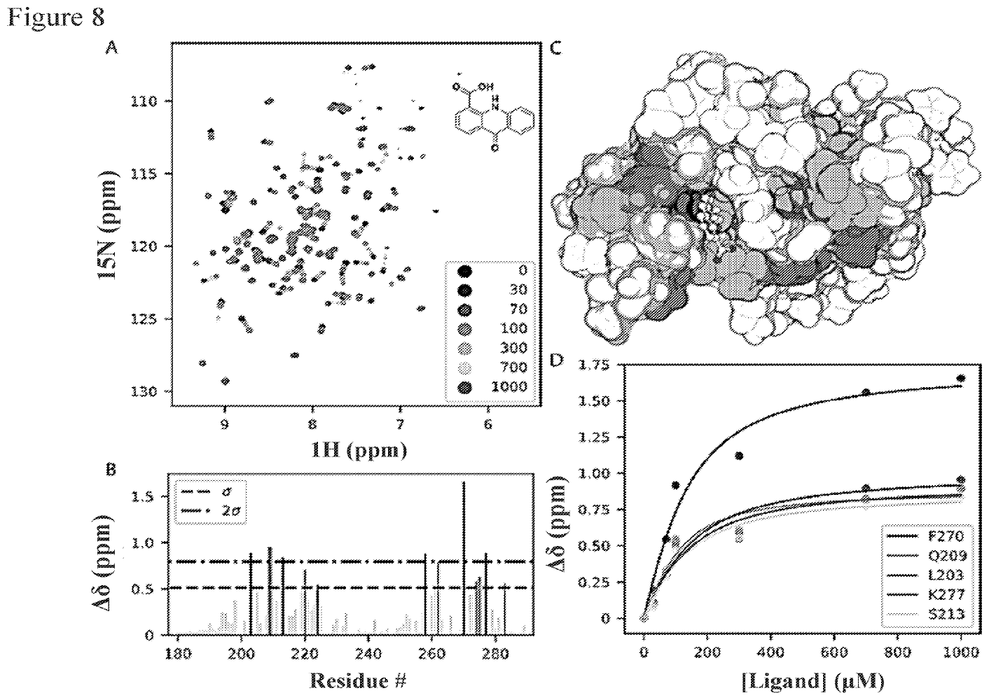

The tightest binding inhibitor was a 2-phenyldihydroquinazolinone (5), giving a K_{d} value of 50 μM (Table 1, S1, FIG. 7), as determined by NMR titrations and ligand efficiency (LE) of 0.32. With a K_{d} value of 60 μM (Table 1, S1, FIG. 8), the second-tightest binder was an acridone scaffold containing carboxylic acid and an LE of 0.33. Fragment binding to PBRM1 was also supported by differential scanning fluorimetry (DSF) with measured positive thermal shifts (Tₘ) of 1±0.2° C. for 5 and 1.5±1.0° C. for 6 (Table 1) against the apoprotein. DSF measures protein unfolding by monitoring the fluorescence of the SYPRO Orange dye as a function of temperature; positive thermal shifts indicate ligand binding induced stabilization of the protein. Given the low molecular weight high potency combined with good ligand efficiency, the dihydroquinazolinone (5) and acridone (6) scaffolds were selected for further optimization.

TABLE 1
Fragment hits identified in the protein-detected NMR screen

| | | | PBRM1- |
|---|---|---|---|
| | | | BD2 |
| | K_{d} | | ΔTₘ |
| Fragments | (μM) | LE^{b} | (° C.)^{a} |
| 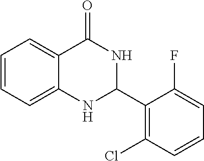 [SMILES: O=C1NC(c2c(F)cccc2Cl)Nc2ccccc21] | 45.3 ± 8.1 | 0.32 | 1.0 ± 0.2 |
| 5 | | | |
| 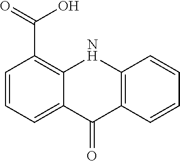 [SMILES: O=C(O)c1cccc2c(=O)c3ccccc3[nH]c12] | 79.1 ± 34.9 | 0.33 | 1.5 ± 1.0 |
| 6 | | | |
| 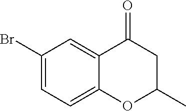 | 170.0 ± 35.0 | 0.4 | 1.0 ± 0.0 |
| 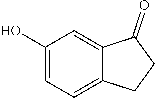 [SMILES: O=C1CCc2ccc(O)cc21] | 902.5 ± 270.5 | 0.4 | ND^{c} |
| 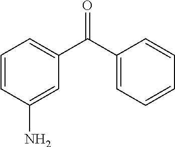 [SMILES: Nc1cccc(C(=O)c2ccccc2)c1] | 708.8 ± 211.4 | 0.28 | ND |
| 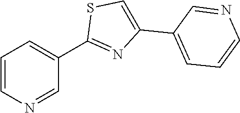 [SMILES: c1cncc(-c2csc(-c3cccnc3)n2)c1] | 1387.2 ± 262.0 | 0.25 | ND |
| 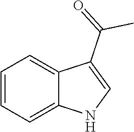 | 1194.9 ± 250 | 0.35 | ND |
| 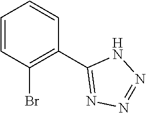 [SMILES: Brc1ccccc1-c1nnn[nH]1] | 1446.2 ± 599 | 0.32 | ND |
| 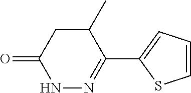 | 1210.0 ± 267.0 | 0.3 | ND |
| 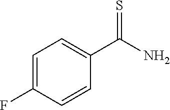 | >2000 | N/A | ND |
| 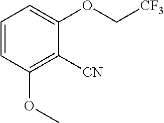 [SMILES: COc1cccc(OCC(F)(F)F)c1C#N] | >2000 | N/A | ND |

^{a}Values are the average of three replicates and standard deviation.
^{b}LE represents Ligand Efficiency
^{c}Tₘ shift not determined by DSF assay.

Structure-Activity Relationship Studies of the Tightest-Binding Fragment

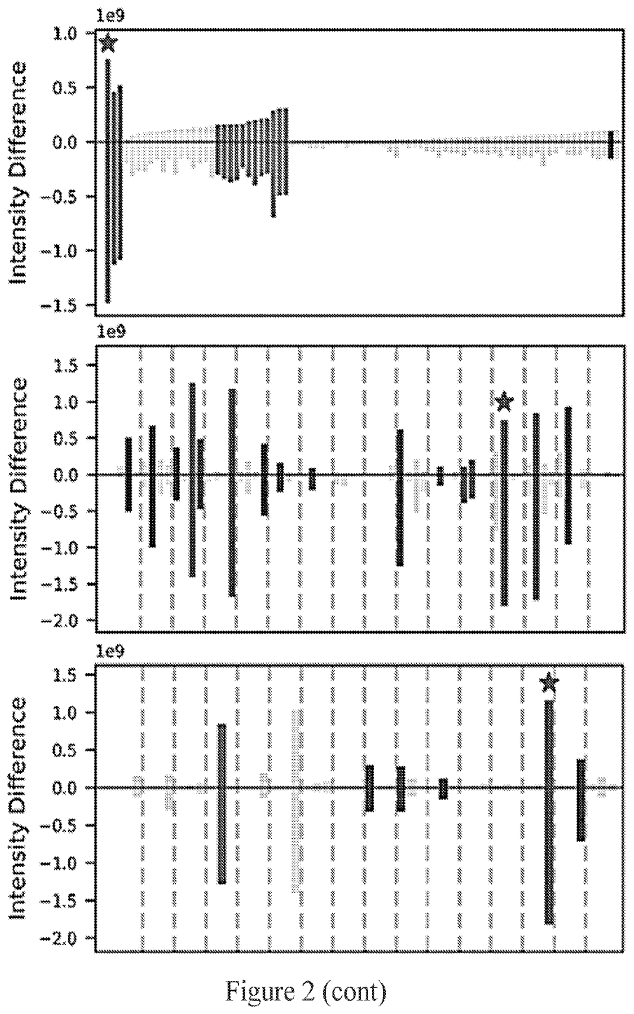

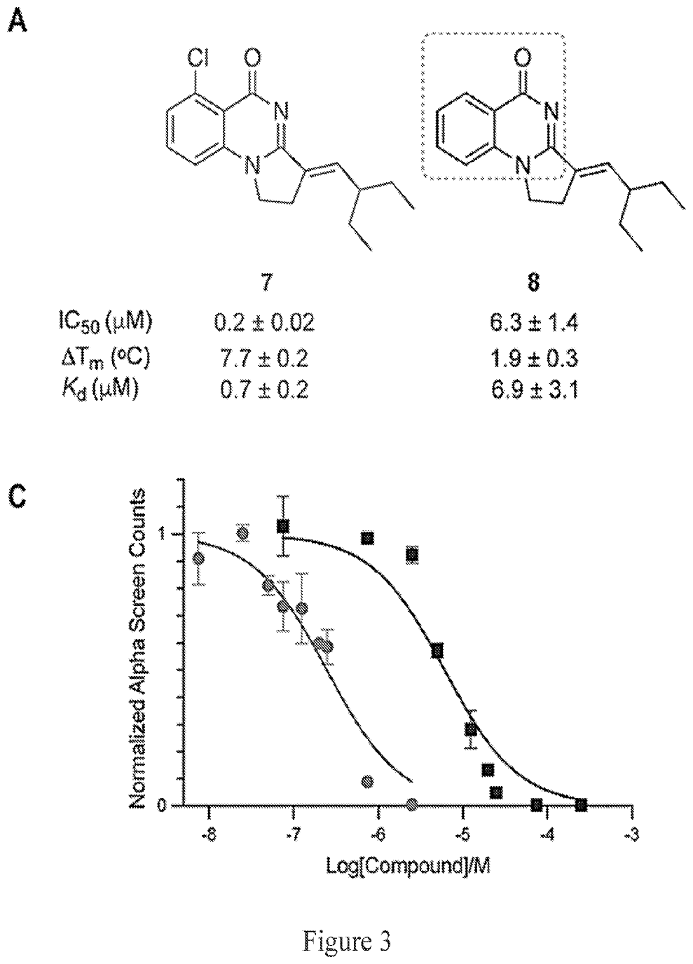

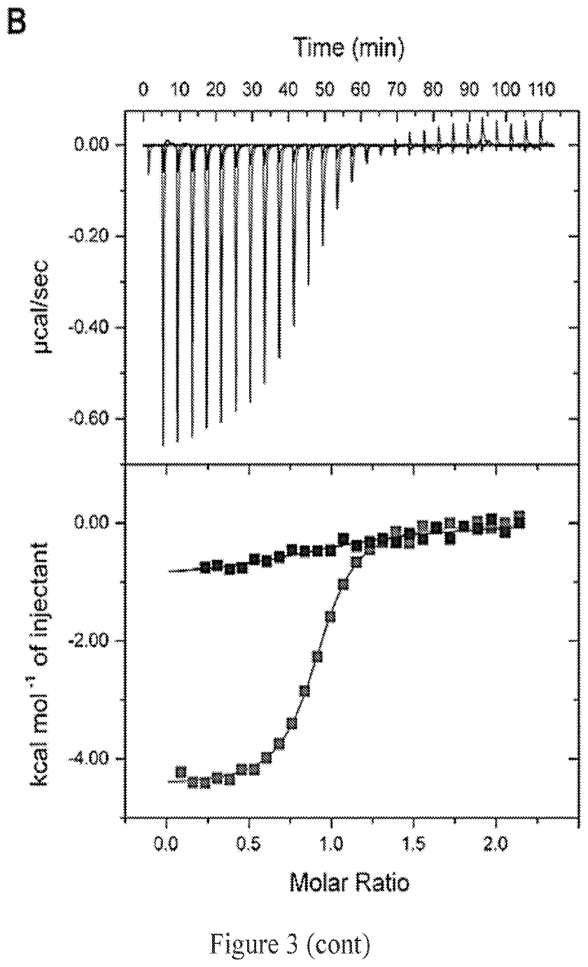

In a previous inhibition study targeting PBRM1-BD5,¹⁷ the introduction of chlorine at the C-5 position of quinazolinone scaffold (highlighted in box, FIG. 3A) improved the binding affinity of the inhibitor towards PBRM1-BD5 by at least 10-fold. On co-crystallization of compound 2 with PBRM1-BD5, the chlorine of 2 was observed to form a halogen bond with the backbone carbonyl of Met731.¹⁷ The homologous residue is Leu255 in PBRM1-BD2. Superposition of PBRM1-BD5 bound to 2 (PDB ID 5FH7; quinazolinone scaffold) and PBRM1-BD2 bound to 4 (PDB ID 6ZN6; aminopyridazine scaffold) indicated the backbone carbonyl of Leu255 is within 3 Å of the chlorine of 2. Therefore, we hypothesized that PBRM1-BD2 would form a similar halogen bond between the chlorine on the quinazolinone scaffold and the carbonyl oxygen of Leu255 of PBRM1-BD2.

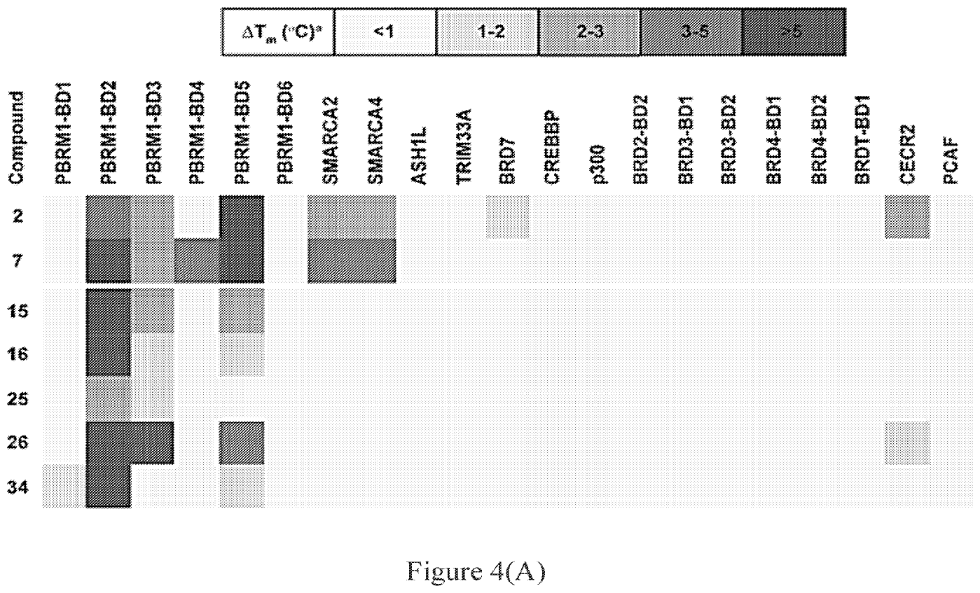

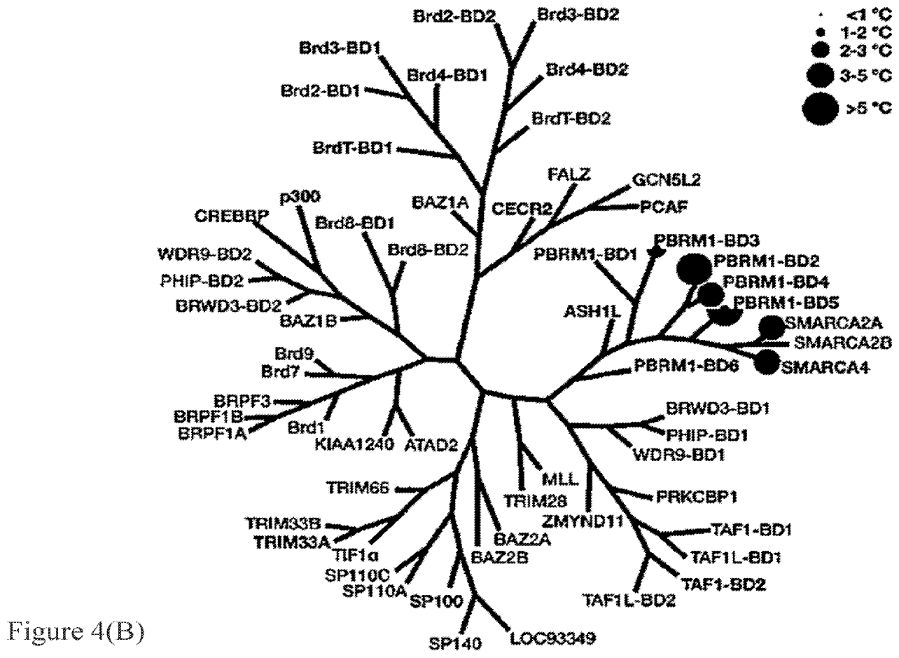

To validate this hypothesis experimentally, we resynthesized 7 and its non-chlorine analog 8 following a published synthetic route (Scheme 2);¹⁷ compound 7 was previously reported to bind PBRM1-BD2 based on a DSF assay;¹⁷ the authors only reported DSF analyses of 7 without further characterization or development of this compound. One reason could be the promiscuity of 7 as it also showed a positive Tₘ shift of >2.5° C. against SMARCA2A/2B/4A bromodomains in DSF assays. The in vitro inhibitory activity of 7 and 8 was tested using our optimized competition-based amplified luminescent proximity homogeneous assays (AlphaScreen), a bead-based assay to quantify biomolecular interactions. In this AlphaScreen assay, the interaction between a biotinylated histone H3K14acetyl peptide and His₆-tagged PBRM1-BD2 brings the streptavidin-coated donor and the Ni²⁺-chelated acceptor beads in close proximity, increasing the luminescence signal; inhibition of this bromodomain:acetyl-lysine interaction via small molecules was quantified by the loss in luminescence signal intensity in a concentration-dependent manner. A >30-fold increase in potency of 7 (IC₅₀=0.2±0.02 μM, FIGS. 3C & 18) relative to 8 (IC₅₀=6.3±1.4 μM, FIGS. 3C & 18) was observed towards PBRM1-BD2. Isothermal titration calorimetry (ITC) was used to further assess the binding affinity of the compounds to the bromodomains of PBRM1-BD2, PBRM1-BD5, and SMARCA4, giving K_{d} values of 0.69 μM, 0.35 μM, and 5.03 μM for 7; and 6.89 μM, 3.26 μM, and no apparent binding for 8 (FIG. 3B, Table 3), respectively. In DSF using the PBRM1-BD2, PBRM1-BD5, SMARCA2, and SMARCA4 bromodomains, 7 showed a positive Tₘ shift of 7.7° C., 11.0° C., 3.0° C., and 3.1° C. (FIG. 4A, Table S2) against the apoprotein, respectively. Compounds 7 and 8 did not show binding to the ASH1L bromodomain, as evaluated by DSF and ITC (FIG. 4A, Table 3, S2). Overall, the binding affinity/potency towards PBRM1-BD2 improved by at least 10-fold by introducing chlorine at the C-5 position of the quinazolinone scaffold.

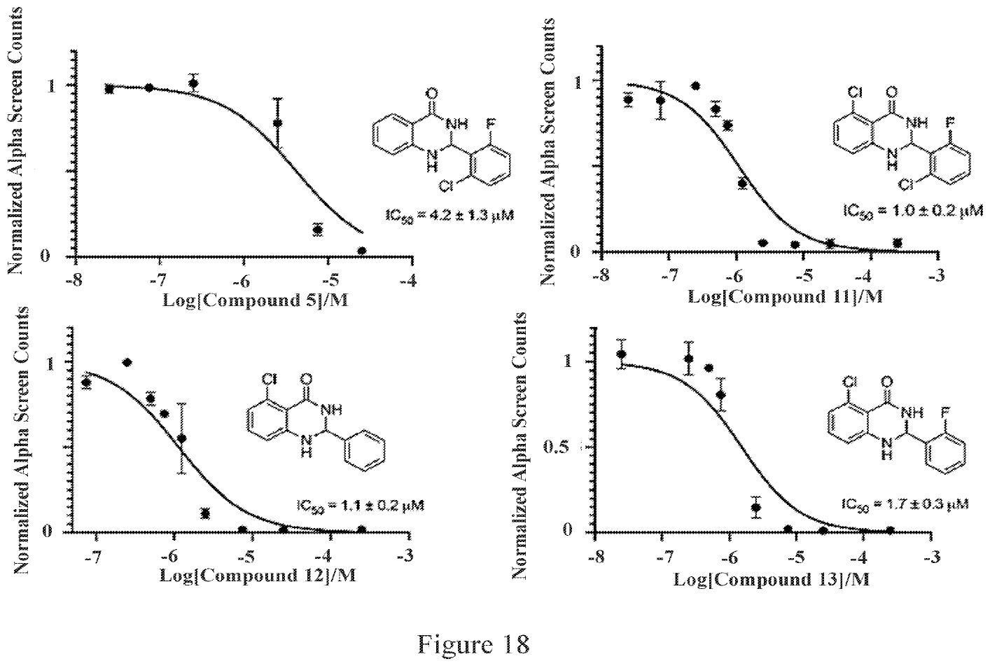

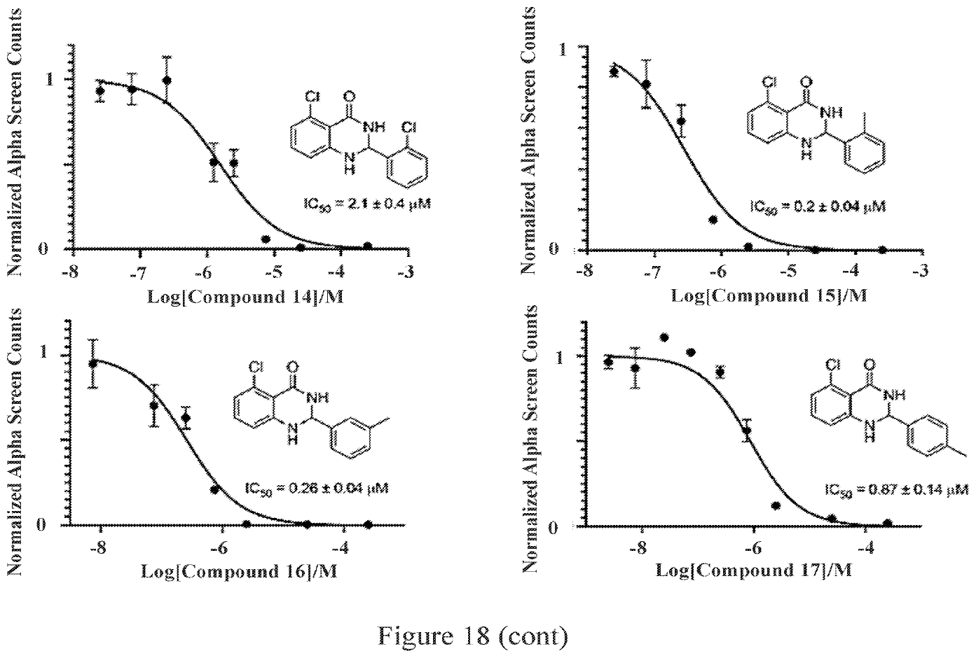

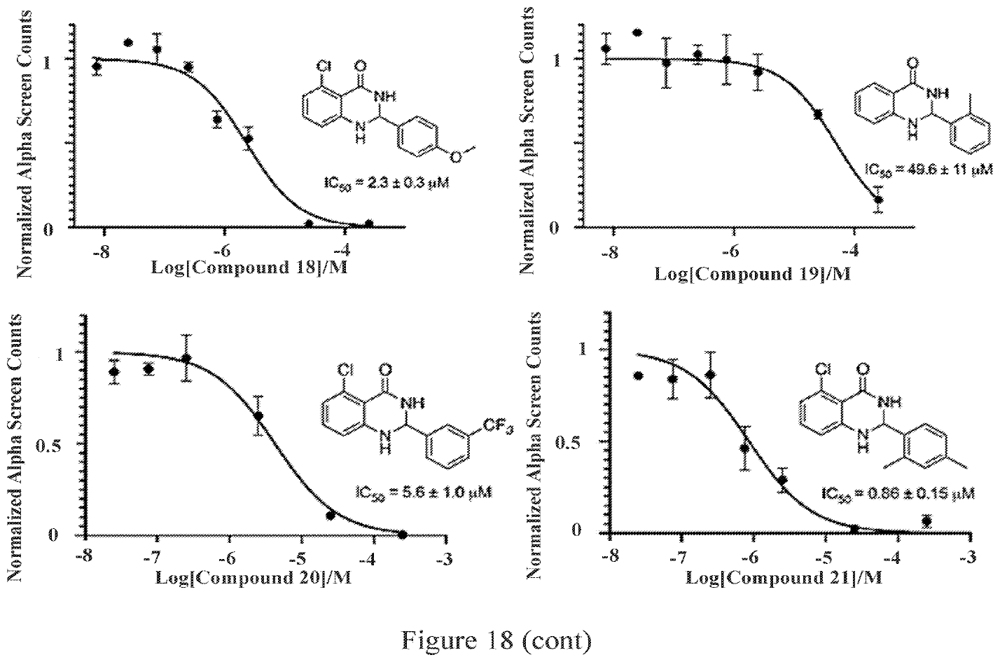

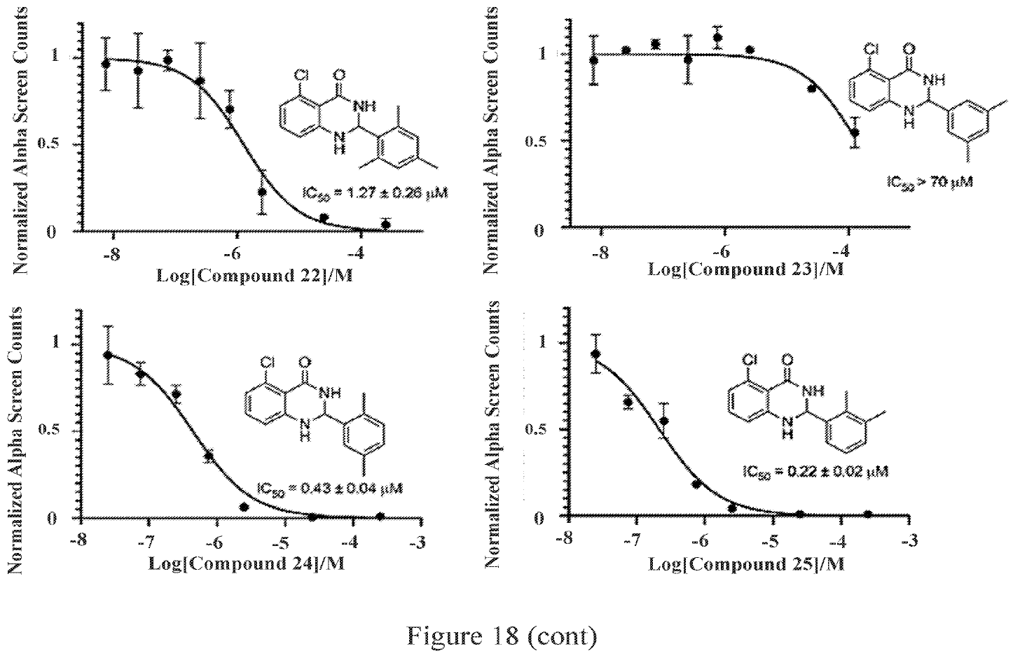

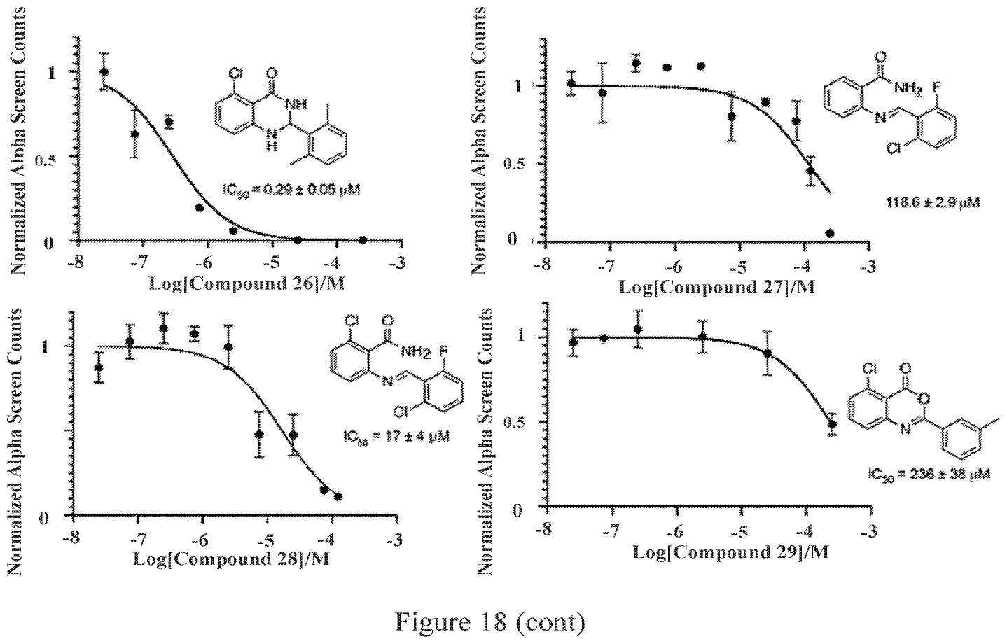

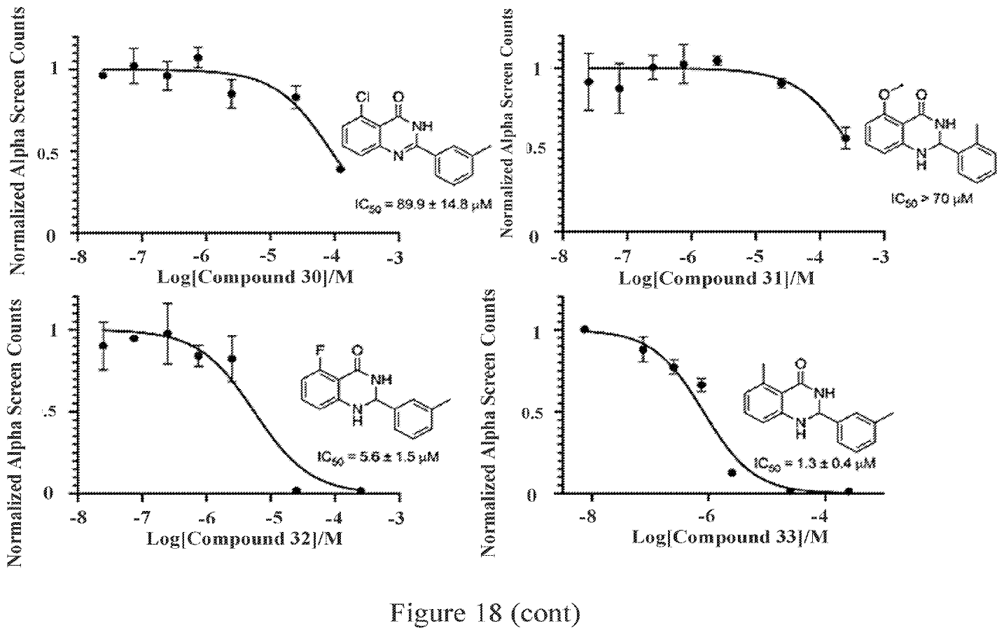

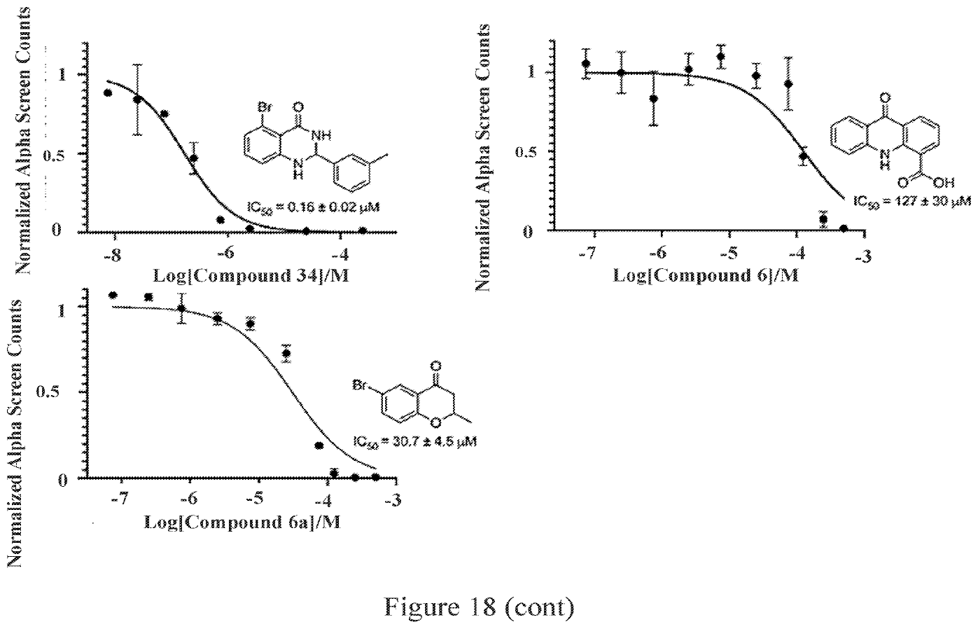

Based on the halogen bond observed in the previous cocrystal structure of PBRM1-BD5 bound to 2¹⁷ and our binding studies, SAR studies of our top hit (5) were initiated by first introducing chlorine to the C-5 position. To access analogs of the dihydroquinazolinone scaffold, the synthetic route depicted in Scheme 1 was used. Basic hydrolysis of 2-aminobenzonitriles (9a-f) resulted in 2-aminobenzamides (10a-f). Condensation of 2-aminobenzamides with requisite benzaldehydes to form dihydroquinazolinones (11-26, 31-34) required base catalysis; absence of base resulted in imine formation (27-28, Scheme 4). Chlorine substitution at the C-5 position of dihydroquinazolinone scaffold of 5 (IC₅₀=4.2±1.3 μM, Table 2, FIG. 18), resulting in 11 (IC₅₀=1.0±0.2 μM, Table 2, FIG. 18), displayed >4-fold increase in potency towards PBRM1-BD2, as determined by AlphaScreen. As assessed by ITC, the binding affinity of 11 to the bromodomains of PBRM1-BD2, PBRM1-BD5, and SMARCA4 was >2-fold tighter than 5, as displayed by K_{d} values of 9.3 μM, 10 μM, 69 μM for 11; and 18.4 μM, 179 μM, 141.6 μM for 5 (Table 3), respectively. Although the potency/binding affinity of 11 was 2-fold better than 5 for PBRM1-BD2, 5 and 11 also bind to bromodomains of SMARCA2/4 (Table 3); thus, inhibitor selectivity within the members of bromodomain family VIII still needed to be addressed.

Scheme 1. General Synthetic Route of Dihydroquinazolinone Scaffold Derivatives

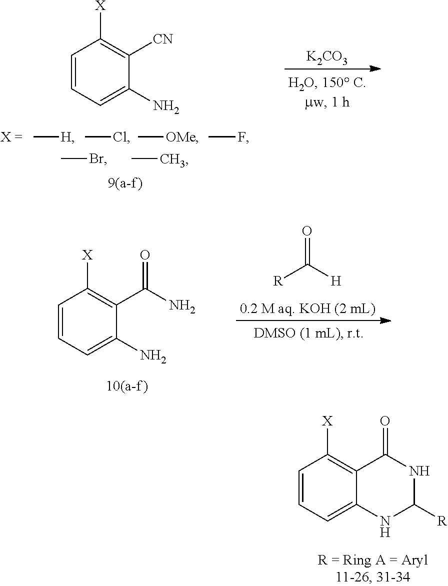

TABLE 2
Inhibition and binding affinity of compounds against
PBRM1-BD2 as tested by AlphaScreen and DSF

| | | | PBRM1-BD2 |
|---|---|---|---|
| Compound ID | Structure | IC₅₀ values (μM)^{a} | ΔTₘ (º C.)^{a} |
| 5 | 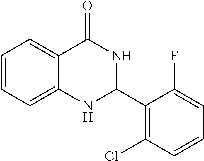 [SMILES: O=C1NC(c2c(F)cccc2Cl)Nc2ccccc21] | 4.2 ± 1.3 | 1.0 ± 0.2 |
| 11 | 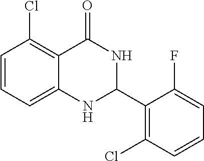 [SMILES: O=C1NC(c2c(F)cccc2Cl)Nc2cccc(Cl)c21] | 1.0 ± 0.2 | 4.4 ± 0.4 |
| 12 | 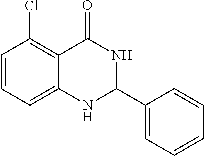 [SMILES: O=C1NC(c2ccccc2)Nc2cccc(Cl)c21] | 1.1 ± 0.2 | 3.8 ± 0.4 |
| 13 | 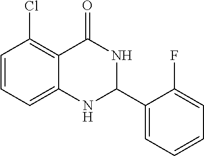 [SMILES: O=C1NC(c2ccccc2F)Nc2cccc(Cl)c21] | 1.7 ± 0.3 | 3.3 ± 0.5 |
| 14 | 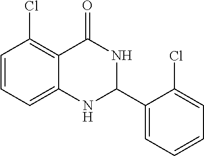 [SMILES: O=C1NC(c2ccccc2Cl)Nc2cccc(Cl)c21] | 2.1 ± 0.4 | 3.7 ± 0.2 |
| 15 | 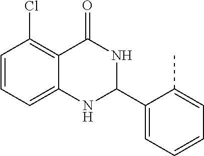 | 0.2 ± 0.04 | 5.4 ± 0.1 |
| 16 | 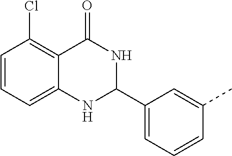 | 0.26 ± 0.04 | 5.4 ± 0.2 |
| 17 | 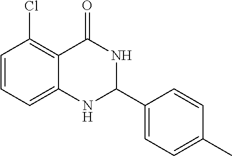 [SMILES: Cc1ccc(C2NC(=O)c3c(Cl)cccc3N2)cc1] | 0.87 ± 0.14 | 2.8 ± 0.2 |
| 18 | 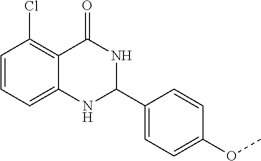 | 2.3 ± 0.3 | 3.4 ± 0.3 |
| 19 | 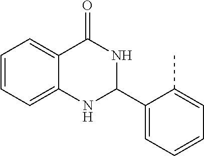 | 49.6 ± 11 | 1.5 ± 0.8 |
| 20 | 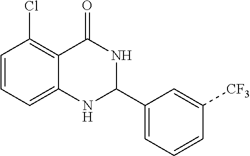 | 5.6 ± 1.0 | 2.9 ± 0.3 |
| 21 | 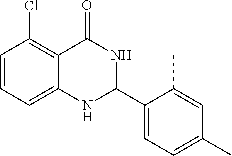 | 0.86 ± 0.15 | 4.5 ± 0.9 |
| 22 | 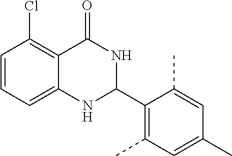 | 1.27 ± 0.26^{b} | 5.5 ± 0.3 |
| 23 | 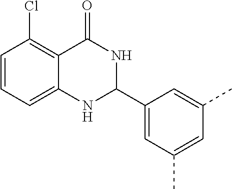 | >70 | 0.23 ± 0.03 |
| 24 | 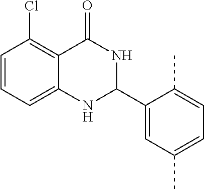 | 0.43 ± 0.04 | 5.4 ± 1.2 |
| 25 | 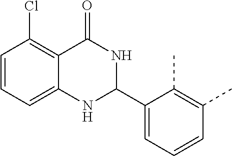 | 0.22 ± 0.02 | 3.9 ± 0.3 |
| 26 | 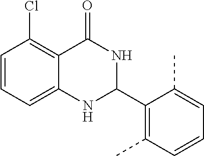 | 0.29 ± 0.05 | 5.2 ± 0.3 |
| 27 | 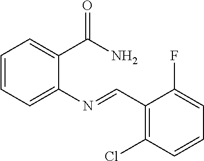 [SMILES: NC(=O)c1ccccc1/N=C/c1c(F)cccc1Cl] | 118.6 ± 2.9 | 0.0 |
| 28 | 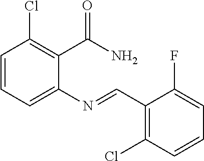 [SMILES: NC(=O)c1c(Cl)cccc1/N=C/c1c(F)cccc1Cl] | 17.0 ± 4.0 | 0.3 ± 0.2 |
| 29 | 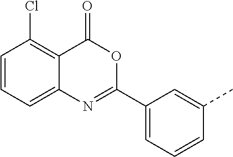 | >70 | 0.0 |
| 30 | 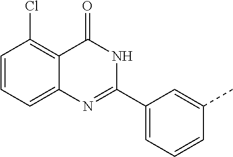 | >70 | 0.0 |
| 31 | 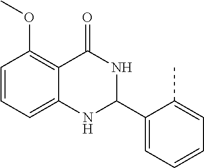 | >70 | 0.0 |
| 32 | 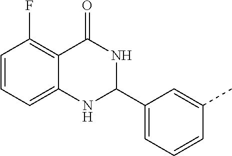 | 5.6 ± 1.5 | 1.8 ± 0.2 |
| 33 | 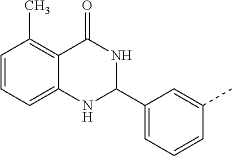 | 1.3 ± 0.4 | 2.6 ± 0.3 |
| 34 | 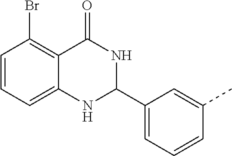 | 0.16 ± 0.02 | 6.4 ± 0.2 |

^{a}Values shown are the average of three replicates and standard deviation.
^{b}Compound was not soluble at tested concentrations.

SAR Studies of the Phenyl Ring Substituted at the 2-Position of the Quinazolinone Fragment

Analogs 12-14 were synthesized to investigate the importance of halogen substitution on phenyl ring substituted at the 2-position (here on referred to as ring A; Scheme 1) of quinazolinone scaffold of 5. The inhibitory potency of this series (12, IC₅₀=1.1±0.2 μM; 13, IC₅₀=1.7±0.3 μM; 14, IC₅₀=2.1±0.4 μM; Table 2, FIG. 18) towards PBRM1-BD2, as determined by the AlphaScreen, was found equivalent to 11 (IC₅₀=1.0±0.2 μM), suggesting the halogens substitution on ring A of 11 does not result in significant improvements in potency towards PBRM1-BD2. Also, in DSF assays using PBRM1-BD2, 12-14 showed similar positive Tₘ shifts (Table 2) to 11, corroborating the AlphaScreen results.

Based on the similarity in the structures of second and fifth bromodomains of PBRM1, we hypothesized that the carbonyl of the dihydroquinazolinone scaffold interacts with the conserved Asn263 of PBRM1-BD2 and the residues on the ZA and BC loops form a hydrophobic pocket. Therefore, we evaluated the introduction of methyl groups at different positions of ring A, resulting in the synthesis of compounds 15-17. Substitution of a methyl group at ortho- or meta-position of ring A, as in 15 (IC₅₀=0.2±0.04 μM; Table 2, FIG. 18) and 16 (IC₅₀=0.26±0.04 μM; Table 2, FIG. 18), resulted in at least 10-fold improvement in PBRM1-BD2 inhibition, as determined by AlphaScreen, consistent with a ‘magic methyl’²³ effect. Compound 15 and 16 displayed equivalent inhibition as the previously reported unselective compound 7 (IC₅₀=0.2±0.02 μM; FIGS. 3C & 18). Interestingly, 15 and 16 did not exhibit detectable thermal stabilization or binding affinity towards the SMARCA2/4 bromodomains as assessed by DSF and ITC (FIG. 4A, Table 3, S2), respectively. Substitutions of methyl or methoxy at the para position of ring A, as in 17 (IC₅₀=0.87±0.14 μM, figure and tables referenced earlier) and 18 (IC₅₀=2.3±0.3 μM), did not improve PBRM1-BD2 inhibition with 17 having potency equivalent to the unsubstituted phenyl analog 11 (IC₅₀=1.0±0.2 μM). Compound 19 (IC₅₀=49.6±11.0 μM), an analog of 15 without the —C₁ substitution at the C-5 position, showed a decrease in potency towards PBRM1-BD2 relative to 15 (IC₅₀=0.2±0.04 μM), emphasizing the importance of halogen bond formation. Compound 20 (IC₅₀=5.6±1.0 μM), with substitution of trifluoromethyl at the meta-position of ring A, resulted in 12-fold decrease in potency relative to the meta methyl analog 16 (IC₅₀=0.26±0.04 μM) towards PBRM1-BD2. In DSF assays using PBRM1-BD2, 15 and 16 showed positive Tₘ shift of >5.2° C. while 17 and 18 showed Tₘ shifts of <4.0° C. relative to apoprotein, consistent with the AlphaScreen results. As assessed by ITC, compound 16 exhibited K_{d} values of 1.5 μM, 43.67 μM, and no apparent binding (FIG. 4D, Table 3) towards PBRM1-BD2, PBRM1-BD5, and SMARCA4, respectively. The binding affinity of 16 (K_{d}=1.5 μM) towards PBRM1-BD2 was equivalent to 7 (K_{d}=0.69 μM), within error, and 16 did not display apparent binding to the SMARCA2/4 bromodomains, as assessed by ITC and DSF (Table 3, FIG. 4A, 4D); 16, to the best of our knowledge, is the most selective inhibitor of PBRM1 reported in the literature and serves as an excellent starting point for the further design of selective PBRM1 chemical probes.

TABLE 3
Binding affinities of selected PBRM1-BD2 inhibitor

| | K_{d} (μM)^{a} | | | | |
|---|---|---|---|---|---|
| Compound | PBRM1-BD2 | PBRM1-BD5 | SMARCA2 | SMARCA4 | ASH1L |
| 5 | 18.4 ± 3.67 | 179 ± 92.34 | 15.90 ± 9.73 | 141.60 ± 151.23 | NB |
| 7 | 0.69 ± 0.15 | 0.35 ± 0.04 | 8.06 ± 4.35 | 5.03 ± 0.67 | NB |
| 8 | 6.89 ± 3.06 | 3.26 ± 0.36 | ND | NB | NB |
| 11 | 9.30 ± 1.15 | 10.10 ± 1.23 | 18.42 ± 9.90 | 69.00 ± 2.09 | NB |
| 16 | 1.48 ± 0.90 | 3.94 ± 2.59 | NB | NB | ND |
| 34 | 4.39 ± 1.32 | 24.70 ± 17.08 | NB | NB | ND |

^{a}K_{d} values for each compound were obtained from ITC measurements and calculated by fitting the data points to a sigmoidal curve. The errors were calculated by standard deviation. ND indicates that the value was not determined. NB indicates the compound did not bind to the bromodomain, as demonstrated by generated heat below 0.1 μcal per second on the ITC traces.

Encouraged by the inhibition data of 15 and 16, analogs 21-26 with methyl substituents at two or more positions of ring A were synthesized and evaluated for their inhibition of acetyl-lysine histone peptide binding to PBRM1-BD2. Substitution of methyl group at the ortho position(s) of ring A of the para methyl analog 17 (IC₅₀=0.87±0.14 μM), resulting in 21 (IC₅₀=0.86±0.15 μM) and 22 (IC₅₀=1.27±0.26 μM), did not improve potency towards PBRM1-BD2. Compound 22 exhibited solubility issues in the AlphaScreen assay, perhaps resulting in apparent weaker potency towards PBRM1-BD2. 23 (IC₅₀>100 μM), with methyl groups substituted at both meta-positions of ring A, resulted in no detectable binding towards PBRM1-BD2. Compounds 24-26 (24, IC₅₀=0.43±0.04 μM; 25, IC₅₀=0.22±0.02 μM; 26, IC₅₀=0.29±0.05 μM) had similar potencies to 16 (IC₅₀=0.26±0.04 μM) against PBRM1-BD2, as determined by AlphaScreen and did not display improvements in IC₅₀ values, potentially because the concentration of PBRM1-BD2 used in the AlphaScreen assay was 0.2 μM and therefore we may have reached the detection limit of the AlphaScreen assay. In DSF assays using PBRM1-BD2, 21-26 (except 23) showed positive Tₘ shifts of >3.5° C. relative to apoprotein. Through these SAR studies of the phenyl ring, we obtained a series of inhibitors (11-26; except 19, 20, and 23) that exhibited up to 35-fold higher potency than the initial fragment hit, 5, identified by the protein-detected NMR screen.

SAR Studies of the Dihydropyrimidone Ring of the Quinazolinone Scaffold

We next investigated the importance of the dihydropyrimidinone ring for the potency of the compounds against PBRM1-BD2. The ring-opened analog of 5 (IC₅₀=4.2±1.3 μM; Scheme 4), compound 27 (IC₅₀>70 μM), and the ring-opened analog of 11 (IC₅₀=1.0±0.2 μM), compound 28 (IC₅₀=17.0±4.0 μM), displayed >16-fold decrease in potency towards PBRM1-BD2, however 28 (IC₅₀=17.0±4.0 μM) was ˜7-fold more potent than 27 (IC₅₀=118.6±2.9 μM), further validating the halogen bond formation hypothesis. On superposition of PBRM1-BD5 bound to 2 (PDB ID 5FH7) and PBRM1-BD2 bound to 4 (PDB ID 6ZN6), the N-3 nitrogen of the quinazolinone scaffold of 2 was found proximal to the carbonyl oxygen of Asn263 with a potential for hydrogen bond formation. Compound 29 (IC₅₀>70 μM) with benzoxazinone scaffold (Scheme 5) did not show inhibition of PBRM1-BD2, supporting the involvement of 16 amide —NH in hydrogen bonding with Asn263. Compound 30 (Scheme 5) was synthesized to investigate the effect of the planarity/aromaticity of the quinazolinone scaffold on the inhibition activity of compound 16 towards PBRM1-BD2. Compound 30 (IC₅₀>70 μM) manifested loss of inhibition of PBRM1-BD2, as determined by AlphaScreen. Also, in DSF assay using PBRM1-BD2, compounds 27, 28, 29, and 31 (Table 2) did not show thermal stabilization compared to apoprotein, corroborating our AlphaScreen results. Hence, we concluded that the dihydropyrimidinone ring is critical for the potency of the compounds towards PBRM1-BD2.

SAR Studies at the C-5 Position of Quinazolinone Scaffold of Compound 5

As detailed above, chlorine introduction at the C-5 position of 5 (IC₅₀=4.2±1.3 μM), resulting in 11 (IC₅₀=1.0±0.2 μM), resulted in a 4-fold increase in potency towards PBRM1-BD2. We further evaluated the best substituent at the C-5 position of the dihydroquinazolinone scaffold; thus, analogs 31-34 were synthesized. Methoxy substitution at C-5 (31, IC₅₀>70 μM) proved detrimental to potency towards PBRM1-BD2, relative to 15 (IC₅₀=0.20±0.04 μM), which could be explained by a potential steric clash between the larger methoxy group and the amide backbone of the protein or a loss in halogen bonding. Substitution of methyl at the C-5 position (33, IC₅₀=1.3±0.4 μM) was preferred over fluorine (32, IC₅₀=5.6±1.5 μM) for inhibition activity towards PBRM1-BD2; bromine substitution at the C-5 position (34, IC₅₀=0.16±0.02 μM) showed equivalent potency to 16 (IC₅₀=0.26±0.04 μM) towards PBRM1-BD2, as determined by AlphaScreen. Thus, chlorine and bromine at the C-5 position of quinazolinone were profitably tolerated to improve potency and were suspected of being involved in halogen bonding with the amide backbone of PBRM1 fluorine substitution did not improve the potency supporting the previous report that fluorine does not form halogen bond.²⁴ In DSF assays using PBRM1-BD2, 34 showed a positive Tₘ shift of >6.2° C., 31 did not show thermal stabilization as expected, 32-33 showed a positive Tₘ shift of >1.5° C. towards apoprotein, consistent with our AlphaScreen results.

Selectivity Profiling of Potent Compounds Against Other Bromodomains

Using DSF assays, we evaluated the selectivity profile of selected compounds exhibiting DTₘ>4° C. towards PBRM1-BD2 against a panel of 22 bromodomains representatives of eight bromodomain subfamilies. We tested 8 bromodomains from family VIII including all six PBRM1 bromodomains, SMARCA2, and SMARCA4; ASH1L from family VII; TRIM33A from family VI; SP100 from family V was found to be unstable at 25° C. and did not provide viable DSF curves; BRD7 (subunit of the PBAF complex) from family IV; CREBBP and p300 from family III; six members from bromodomain and extra terminal domain (BET) family including BRD2-BD2, BRD3-BD1, BRD3-BD2, BRD4-BD1, BRD4-BD2, and BRDT-BD1 from family II; and PCAF and CECR2 from family I. Compound 15 showed positive Tₘ of 5.4° C. towards PBRM1-BD2 and Tₘ of >2° C. towards PBRM1-BD3 and PBRM1-BD5, and no significant thermal stabilization (<1° C.) against other tested bromodomains (FIG. 4A, Table S3). Compound 16 showed a positive Tₘ shift of 5.4° C. towards PBRM1-BD2 and moderate Tₘ shifts of 1.8° C. towards PBRM1-BD3 and PBRM1-BD5 (FIG. 4A, Table S3). Compound 26 was broadly selective for PBRM1 depicting a Tₘ shift of 5.2° C. for PBRM1-BD2 and a Tₘ shift of >4° C. for PBRM1-BD3 and PBRM1-BD5 (FIG. 4A, Table S3); compound 26 could be developed to target multi bromodomains of PBRM1. Compound 34 showed an excellent Tₘ shift of 6.4° C. towards PBRM1-BD2 and Tₘ shift of ˜1.5° C. for PBRM1-BD1 and PBRM-BD5 (FIG. 4A, Table S3). In contrast, compound 7 showed a Tₘ shift of >7.5° C. for PBRM1-BD2 and PBRM1-BD5 but also a considerable Tₘ shift of >3° C. for the SMARCA2/4 bromodomains (FIG. 4A, Table S3), as reported.¹⁷ In summary, all tested compounds developed herein were exclusively selective to the PBRM1 bromodomains compared to other tested bromodomains; the previously reported 2¹⁷ and 7¹⁷ manifested positive thermal stabilization towards other bromodomains family VIII, as predicted.

Activity of PBRM1-BD2-Selective Inhibitors Against PBRM1-Dependent Prostate Cancer Cells.

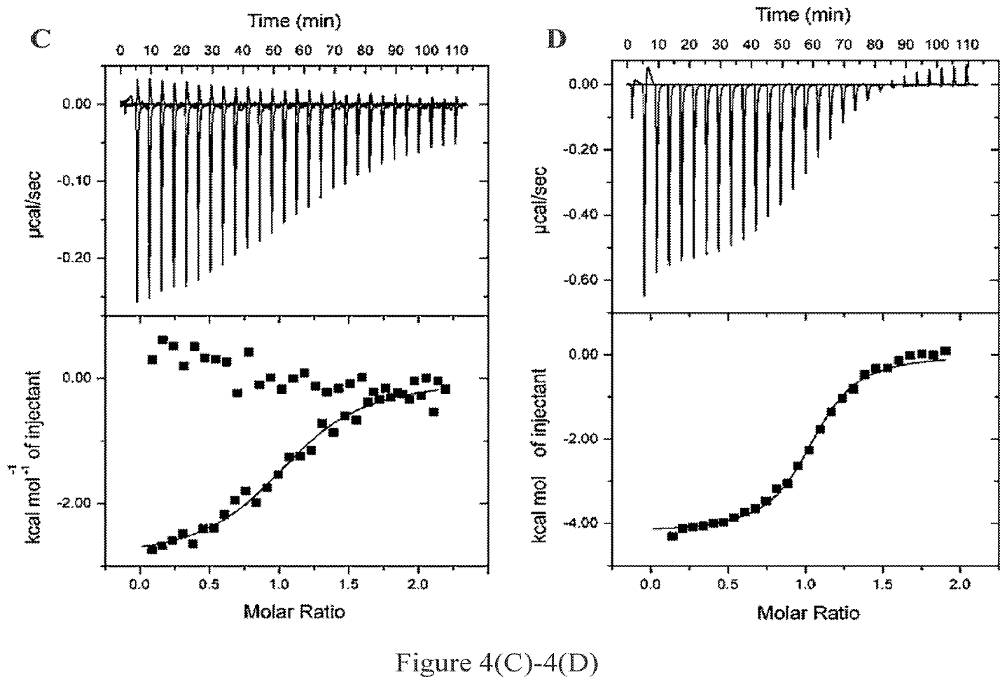

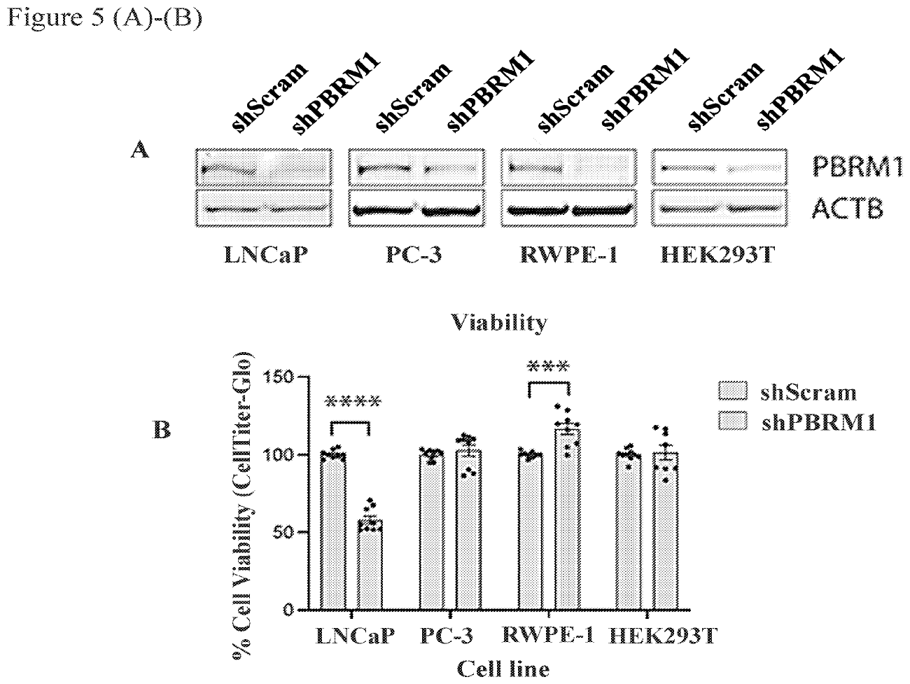

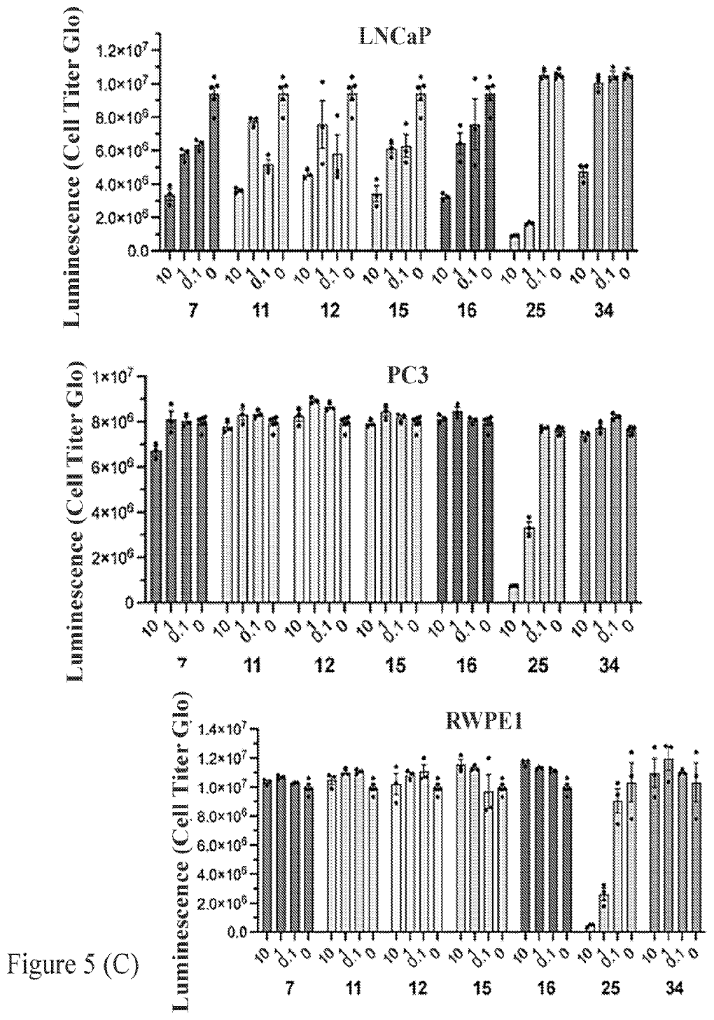

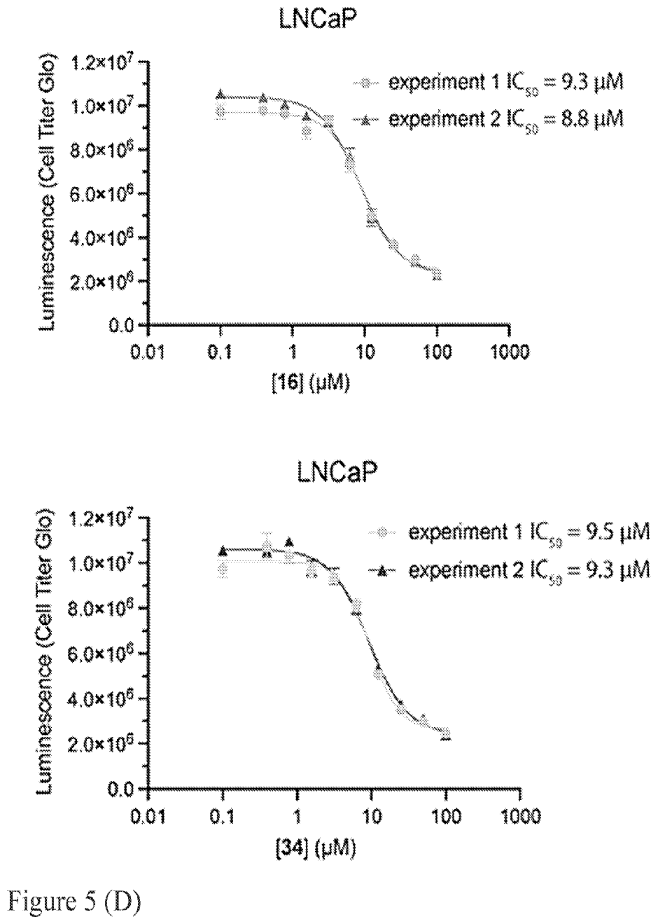

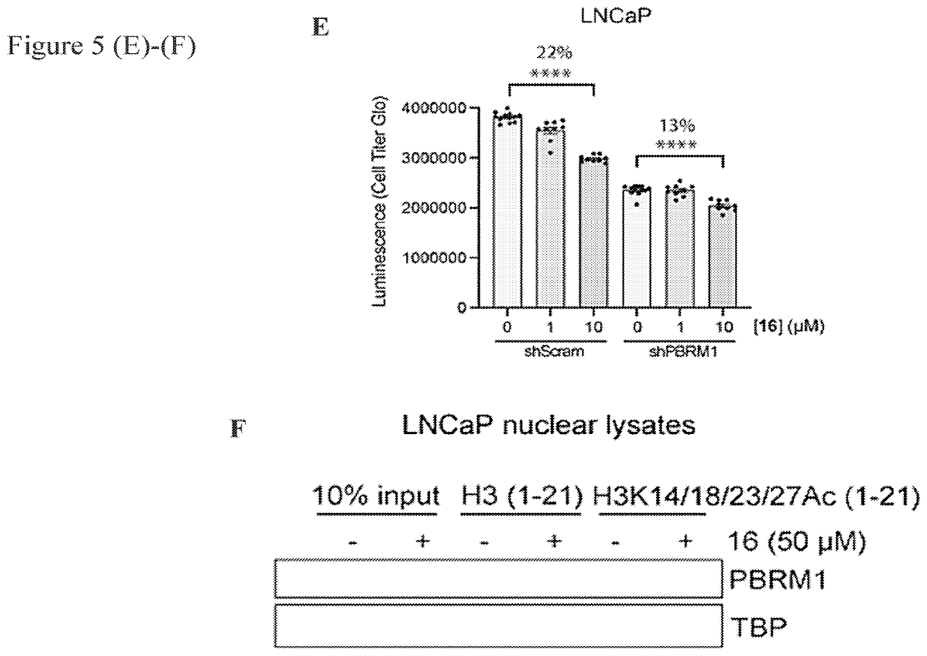

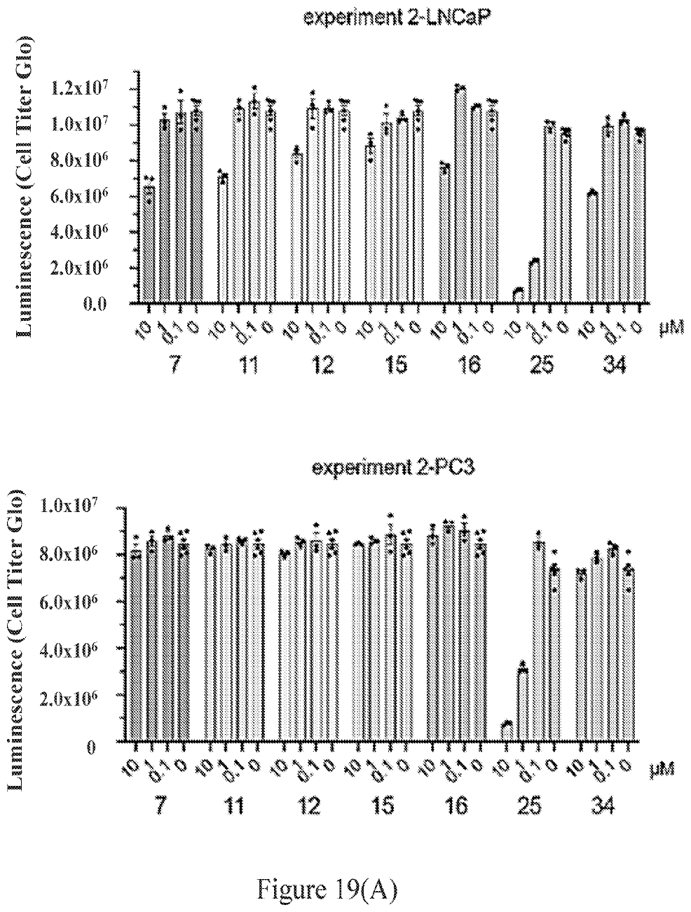

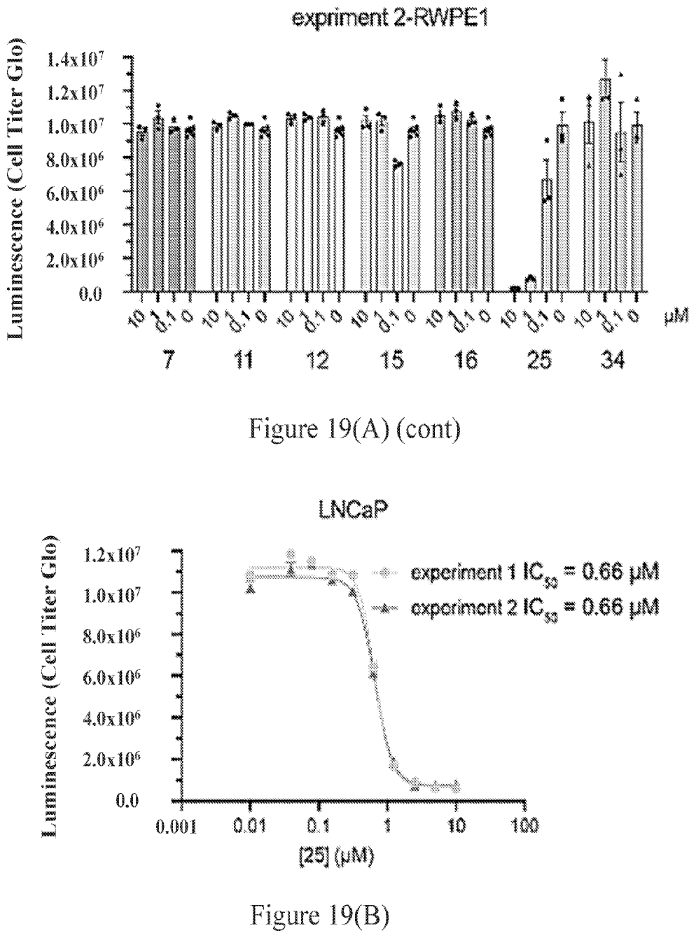

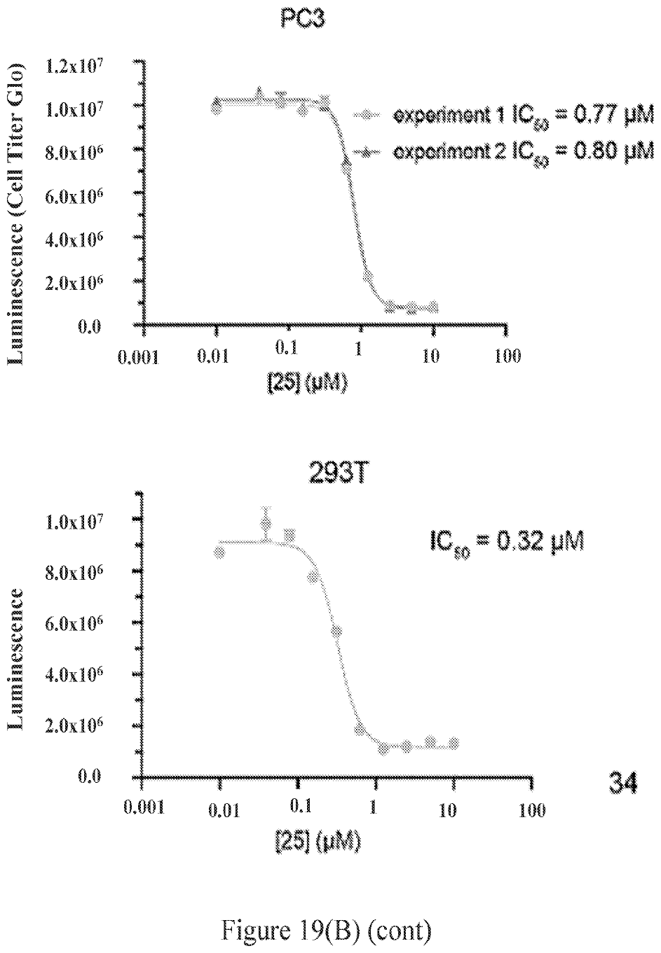

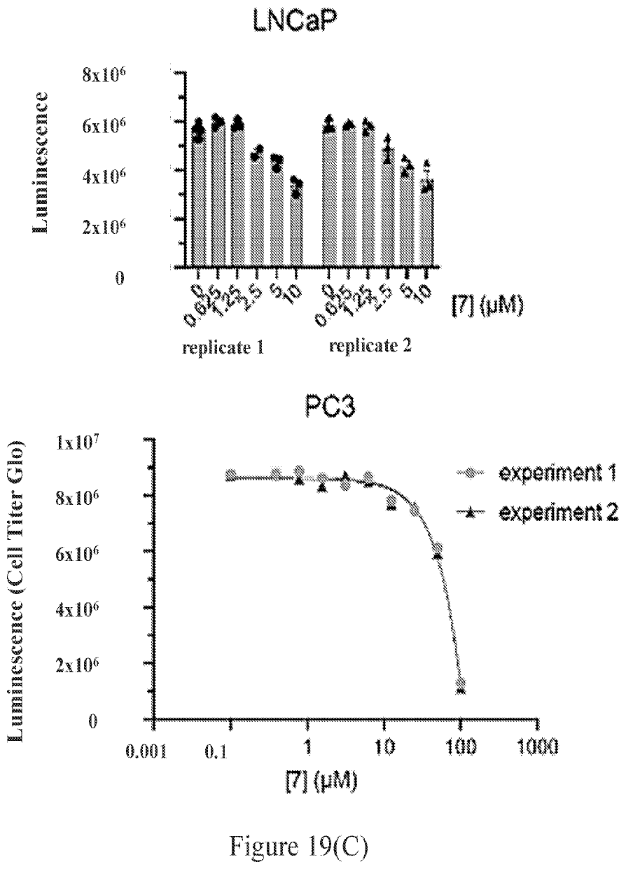

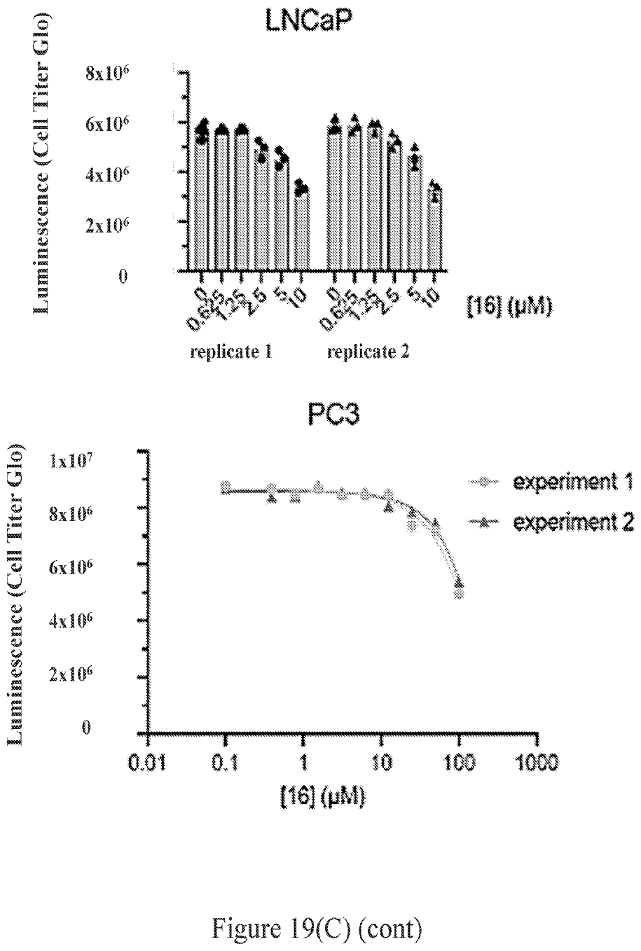

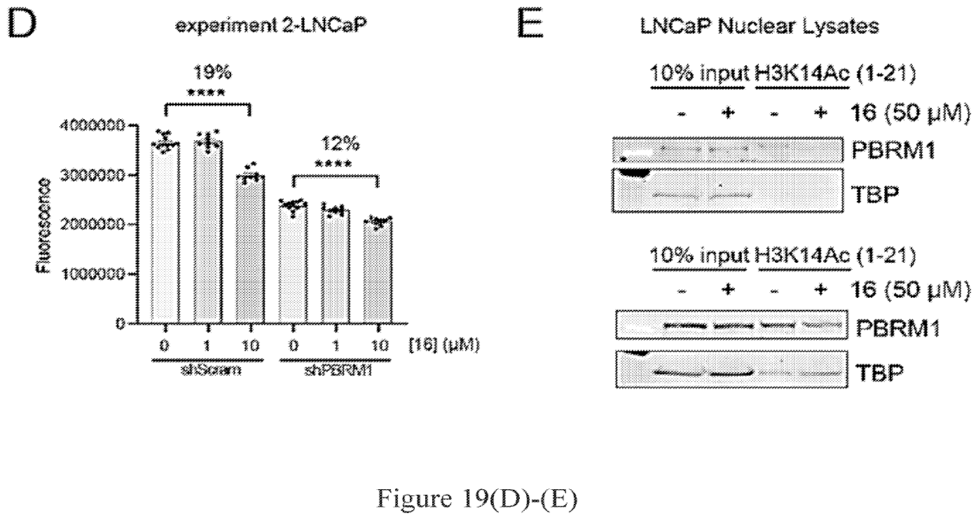

To determine which compound would make the best chemical probe for use in a cellular context, we needed a cell line selectively dependent on PBRM1 for viability. In prostate cancer patient tumors, increased expression of PBRM1 correlates with aggressiveness¹² and facilitates survival under oxidative stress conditions.^{11, 13} Using lentiviral delivery of shRNA, we determined the viability dependence on PBRM1 for a panel of cell lines including androgen receptor (AR) positive prostate cancer cell line LNCaP (AR negative prostate cancer cell line PC3), prostate epithelial cell line RWPE-1, and embryonic kidney cell line HEK293T (FIG. 5A). The LNCaP cells were the only cell line with decreased viability upon PBRM1 knockdown (FIG. 5A). Since LNCaP cells are highly dependent on AR signaling, the PBRM1 is in line with the correlation between protein expression levels for PBRM1 and AR target PSA in tumors¹² and the decrease in AR target gene expression upon PBAF subunit knockout in a male leukemia line.²⁵ In contrast, the viability of AR-negative prostate cancer cell line PC3 and non-cancerous cell lines RWPE-1 and HEK293T are not reduced with shPBRM1, and in fact, RWPE-1 cells grow slightly faster with shPBRM1, similar to what we have seen in other epithelial cell lines.¹³ Next, we tested the ability of the best PBRM1-BD2 ligands to inhibit the growth of the human prostate cell lines LNCaP, PC3, and RWPE1 at three doses 10, 1, and 0.1 μM (FIGS. 5B & 19A). All of the compounds inhibit LNCaP growth at higher concentrations, consistent with their ability to inhibit PBRM1-BD2. The most potent compound, 25, inhibits LNCaP cells with sub-micromolar potency (IC₅₀=0.6-0.7 μM) but also inhibits the PC3, RWPE1, and HEK293T cells similarly (FIGS. 5B & 19A/B), indicating off-target toxicity. Based on the biochemical properties and the selectivity and potency against LNCaP cells, the next most promising compounds were 16 and 34, which differ only in the halogen substitution (chlorine for 16 and bromine for 34). Both compounds have similar potency against LNCaP (IC₅₀s˜ 9 μM) (FIG. 5C) and do not inhibit the growth of PC3 and RWPE1 at tested concentrations (FIG. 5B). Although compound 7, which also binds to PBRM1-BD5 and SMARCA2/4 BDs, displays similar growth inhibition of LNCaP cells as the BD-selective ligand 16 (FIG. 5B), it also displays more significant inhibition of PBRM1-independent prostate cancer cell line PC3 (FIG. 19C), potentially indicating off-target effects from inhibiting SMARCA2/4, which also contributes to prostate cancer viability through cBAF and GBAF.^{26, 27}

To confirm that the activity of 16 in LNCaP is through PBRM1, we compared the activity of 16 in LNCaP cells with and without PBRM1 knockdown (FIGS. 5D & 19E). We found that 10 μM of 16 has a much more significant effect on LNCaP viability in cells with PBRM1 than in cells with PBRM1 knockdown, implicating on-target action. We also confirmed that 16 could reduce the association of acetylated substrates to full-length PBRM1 in PBAF. The addition of PBRM1-BD2 inhibitor 16 LNCaP lysates reduces the association of WT PBRM1 to H₃ peptides with multiple acetylation marks (FIG. 5E) or H3K14Ac alone (FIG. 19E).

SAR Studies on the Second-Tightest Binding Fragment Hit

Preliminary SAR studies on the second-best fragment, 6, consisting of an acridone scaffold, were also performed. Compounds 35-38 (Scheme 6) were synthesized to assess their improvements in potency towards PBRM1-BD2. 36 (IC₅₀=7 μM, Table S3), with chlorine substituted at the C-8 position of acridone ring, manifested a 17-fold increase in potency towards PBRM1-BD2 compared to 6 (IC₅₀=127±30 μM, Table S3), further supporting the hypothesis of chlorine participating in the halogen-bond formation with the protein backbone. Using PBRM1-BD2, 36 exhibited a Tₘ shift of >1.5° C. compared to 6, as assessed by DSF. Compound 35 (IC₅₀=26 μM, Table S3), the ring-opened analog of 36, had at >4-fold decreased potency towards PBRM1-BD2 compared to 36. Introduction of chlorine at the C-1 position of 6, resulting in 38 (IC₅₀>250 μM, Table S3), and ring-opened analog 37 (IC₅₀>250 μM, Table S3), was detrimental to the inhibitory potency of the acridone scaffold towards PBRM1-BD2. Overall, chlorine substitution at the C-8 position of 6 resulted in improved inhibition, and planarity of the acridone scaffold is favored for its inhibition activity towards PBRM1-BD2. We speculate that the presence of a carboxylic acid at the 4′-position of 6 offers a promising handle to develop 6 into a more potent and soluble compound.

### CONCLUSION

We have described SAR studies on a fragment hit identified by a protein-detected NMR screen targeting PBRM1-BD2 and optimized the tightest-binding fragment into a series of cell-active inhibitors that display excellent selectivity towards PBRM1, especially over bromodomains of SMARCA2/4 and other bromodomain-containing proteins. Our sequential SAR studies observed that the modifications on the phenyl ring substituted at the 2-position (ring A) of dihydroquinazolinone scaffold of 5 were more forgiving. The best substituent was a methyl at the meta- and/or ortho-position of ring A. Compounds 15 (IC₅₀=0.2±0.04 μM), 16 (IC₅₀=0.26±0.04 μM), 25 (IC₅₀=0.22±0.02 μM), and 26 (IC₅₀=0.29±0.05 μM), 35 (IC₅₀=0.16±0.02 μM) showed inhibitory activities comparable to the previously described unselective 7 (IC₅₀=0.2±0.02 μM) (Table 2); however, these compounds demonstrated excellent selectivity especially within family VIII bromodomains, as assessed by DSF (FIG. 4A, Table S3). The dihydropyrimidinone ring was critical for the inhibitory activity of compounds towards PBRM1-BD2. Compounds 29 and 30 with IC₅₀>70 μM towards PBRM1-BD2 emphasize the importance of a proposed hydrogen-bond and non-aromatic character of analogs for their inhibition potency. Only bromine of 34 (IC₅₀=0.16±0.02 μM) could replace chlorine at the C-5 position of 16 (IC₅₀=0.26±0.04 μM). Compound 16 showed promising inhibition towards PBRM1-dependent prostate cancer cell line LNCaP, giving an IC₅₀ value of 9 μM. Given its robust cellular activity and favorable pharmaceutical properties, 16 warrants further in vivo studies. Although 15, 16, 25, and 34 exhibited excellent inhibition activity in vitro and in cells (Table 2, FIGS. 5 & 19), future SAR studies will be enabled by obtaining a cocrystal structure of the protein-bound to either 16, which would be useful for further improvements in potency and pharmacological properties.

Overall, multiple lines of evidence detailing selective binding and inhibitory activities of compounds to PBRM1-BD2 were obtained. Importantly, to our knowledge, this study provides the first demonstration of inhibitors with excellent selectivity for PBRM1, making them suitable chemical probes for future biological studies. Given the overall importance of PBRM1 function in development and diseases, the availability of such small-molecule inhibitors provides an important avenue for the further development of novel PBRM1-targeted cancer therapies. Also, the studies correlating the inactivation of the Pbrm1 gene to improvements in anti-PD1 cancer immunotherapy are controversial. The lack of selective chemical probes is partly responsible for the evasion of a detailed study investigating the function of PBRM1 as a tumor repressor or growth enhancer. We envision that using these chemical probes will advance understanding of the underlying biological roles of PBRM1 in health and disease.

### REFERENCES

1. Choudhary, C.; Kumar, C.; Gnad, F.; Nielsen, M. L.; Rehman, M.; Walther, T. C.; Olsen, J. V.; Mann, M. Lysine acetylation targets protein complexes and co-regulates major cellular functions. Science 2009, 325, 834-40.
2. Filippakopoulos, P.; Picaud, S.; Mangos, M.; Keates, T.; Lambert, J.-P.; Barsyte-Lovejoy, D.; Felletar, I.; Volkmer, R.; MWller, S.; Pawson, T.; Gingras, A.-C.; Arrowsmith, H., Cheryl; Knapp, S. Histone Recognition and Large-Scale Structural Analysis of the Human Bromodomain Family. Cell 2012, 149, 214-231.
3. Thompson, M. Polybromo-1: the chromatin targeting subunit of the PBAF complex. Biochimie 2009, 91, 309-319.
4. Cochran, A. G.; Conery, A. R.; Sims, R. J., 3rd. Bromodomains: a new target class for drug development. Nat Rev Drug Discov 2019, 18, 609-628.
5. Jahagirdar, R.; Attwell, S.; Marusic, S.; Bendele, A.; Shenoy, N.; McLure, K. G.; Gilham, D.; Norek, K.; Hansen, H. C.; Yu, R.; Tobin, J.; Wagner, G. S.; Young, P. R.; Wong, N. C. W.; Kulikowski, E. RVX-297, a BET Bromodomain Inhibitor, Has Therapeutic Effects in Preclinical Models of Acute Inflammation and Autoimmune Disease. Mol Pharmacol 2017, 92, 694-706.
6. Singh, M. B.; Sartor, G. C. BET bromodomains as novel epigenetic targets for brain health and disease. Neuropharmacology 2020, 181, 108306.
7. Porter, E. G.; Dykhuizen, E. C. Individual Bromodomains of Polybromo-1 Contribute to Chromatin Association and Tumor Suppression in Clear Cell Renal Carcinoma. J Biol Chem 2017, 292, 2601-2610.
8. Varela, I.; Tarpey, P.; Raine, K.; Huang, D.; Ong, C. K.; Stephens, P.; Davies, H.; Jones, D.; Lin, M.-L.; Teague, J.; Bignell, G.; Butler, A.; Cho, J.; Dalgliesh, G. L.; Galappaththige, D.; Greenman, C.; Hardy, C.; Jia, M.; Latimer, C.; Lau, K. W.; Marshall, J.; Mclaren, S.; Menzies, A.; Mudie, L.; Stebbings, L.; Largaespada, D. A.; Wessels, L. F. A.; Richard, S.; Kahnoski, R. J.; Anema, J.; A. Tuveson, D.; Perez-Mancera, P. A.; Mustonen, V.; Fischer, A.; Adams, D. J.; Rust, A.; Chan-On, W.; Subimerb, C.; Dykema, K.; Furge, K.; Campbell, P. J.; Teh, B. T.; Stratton, M. R.; Futreal, P. A. Exome sequencing identifies frequent mutation of the SWI/SNF complex gene PBRM1 in renal carcinoma. Nature 2011, 469, 539-542.
9. Pan, D.; Kobayashi, A.; Jiang, P.; Ferrari De Andrade, L.; Tay, R. E.; Luoma, A. M.; Tsoucas, D.; Qiu, X.; Lim, K.; Rao, P.; Long, H. W.; Yuan, G.-C.; Doench, J.; Brown, M.; Liu, X. S.; Wucherpfennig, K. W. A major chromatin regulator determines resistance of tumor cells to T cell-mediated killing. Science 2018, 359, 770-775.
10. Miao, D.; Margolis, C. A.; Gao, W.; Voss, M. H.; Li, W.; Martini, D. J.; Norton, C.; Bosse, D.; Wankowicz0, S. M.; Cullen, D.; Horak, C.; Wind-Rotolo, M.; Tracy, A.; Giannakis, M.; Hodi, F. S.; Drake, C. G.; Ball, M. W.; Allaf, M. E.; Snyder, A.; Hellmann, M. D.; Ho, T.; Motzer, R. J.; Signoretti, S.; Kaelin, W. G.; Choueiri, T. K.; Van Allen, E. M. Genomic correlates of response to immune checkpoint therapies in clear cell renal cell carcinoma. Science 2018, 359, 801-806.
11. Hagiwara, M.; Fushimi, A.; Yamashita, N.; Bhattacharya, A.; Rajabi, H.; Long, M. D.; Yasumizu, Y.; Oya, M.; Liu, S.; Kufe, D. MUC1-C activates the PBAF chromatin remodeling complex in integrating redox balance with progression of human prostate cancer stem cells. Oncogene 2021, 40, 4930-4940.
12. Mota, S. T. S.; Vecchi, L.; Zóia, M. A. P.; Oliveira, F. M.; Alves, D. A.; Dornelas, B. C.; Bezerra, S. M.; Andrade, V. P.; Maia, Y. C. P.; Neves, A. F.; Goulart, L. R.; Araújo, T. G. New Insights into the Role of Polybromo-1 in Prostate Cancer. International Journal of Molecular Sciences 2019, 20, 2852.
13. Porter, E. G.; Dhiman, A.; Chowdhury, B.; Carter, B. C.; Lin, H.; Stewart, J. C.; Kazemian, M.; Wendt, M. K.; Dykhuizen, E. C. PBRM1 Regulates Stress Response in Epithelial Cells. iScience 2019, 15, 196-210.
14. Fedorov, O.; Castex, J.; Tallant, C.; Owen, D. R.; Martin, S.; Aldeghi, M.; Monteiro, O.; Filippakopoulos, P.; Picaud, S.; Trzupek, J. D.; Gerstenberger, B. S.; Bountra, C.; Willmann, D.; Wells, C.; Philpott, M.; Rogers, C.; Biggin, P. C.; Brennan, P. E.; Bunnage, M. E.; Schule, R.; Gunther, T.; Knapp, S.; Miller, S. Selective targeting of the BRG/PB1 bromodomains impairs embryonic and trophoblast stem cell maintenance. Science Advances 2015, 1, e1500723.
15. Gerstenberger, B. S.; Trzupek, J. D.; Tallant, C.; Fedorov, O.; Filippakopoulos, P.; Brennan, P. E.; Fedele, V.; Martin, S.; Picaud, S.; Rogers, C.; Parikh, M.; Taylor, A.; Samas, B.; O'Mahony, A.; Berg, E.; Pallares, G.; Torrey, A. D.; Treiber, D. K.; Samardjiev, I J.; Nasipak, B. T.; Padilla-Benavides, T.; Wu, Q.; Imbalzano, A. N.; Nickerson, J. A.; Bunnage, M. E.; Muller, S.; Knapp, S.; Owen, D. R. Identification of a Chemical Probe for Family VIII Bromodomains through Optimization of a Fragment Hit. J Med Chem 2016, 59, 4800-11.
16. Melin, L.; Gesner, E.; Attwell, S.; Kharenko, O. A.; van der Horst, E. H.; Hansen, H. C.; Gagnon, A. Design and Synthesis of LM146, a Potent Inhibitor of PB1 with an Improved Selectivity Profile over SMARCA2. ACS Omega 2021, 6, 21327-21338.
17. Sutherell, C. L.; Tallant, C.; Monteiro, O. P.; Yapp, C.; Fuchs, J. E.; Fedorov, O.; Siejka, P.; Müller, S.; Knapp, S.; Brenton, J. D.; Brennan, P. E.; Ley, S. V. Identification and Development of 2,3-Dihydropyrrolo[1,2-a]quinazolin-5(1H)-one Inhibitors Targeting Bromodomains within the Switch/Sucrose Nonfermenting Complex. Journal of Medicinal Chemistry 2016, 59, 5095-5101.
18. Myrianthopoulos, V.; Gaboriaud-Kolar, N.; Tallant, C.; Hall, M.-L.; Grigoriou, S.; Brownlee, P. M.; Fedorov, O.; Rogers, C.; Heidenreich, D.; Wanior, M.; Drosos, N.; Mexia, N.; Savitsky, P.; Bagratuni, T.; Kastritis, E.; Terpos, E.; Filippakopoulos, P.; Müller, S.; Skaltsounis, A.-L.; Downs, J. A.; Knapp, S.; Mikros, E. Discovery and Optimization of a Selective Ligand for the Switch/Sucrose Nonfermenting-Related Bromodomains of Polybromo Protein-1 by the Use of Virtual Screening and Hydration Analysis. Journal of Medicinal Chemistry 2016, 59, 8787-8803.
19. Wanior, M.; Preuss, F.; Ni, X.; Kramer, A.; Mathea, S.; Gobel, T.; Heidenreich, D.; Simonyi, S.; Kahnt, A. S.; Joerger, A. C.; Knapp, S. Pan-SMARCA/PB1 Bromodomain Inhibitors and Their Role in Regulating Adipogenesis. J Med Chem 2020, 63, 14680-14699.
20. Charlop-Powers, Z.; Zeng, L.; Zhang, Q.; Zhou, M. M. Structural insights into selective histone H₃ recognition by the human Polybromo bromodomain 2. Cell Res 2010, 20, 529-38.
21. Egner, J. M.; Jensen, D. R.; Olp, M. D.; Kennedy, N. W.; Volkman, B. F.; Peterson, F. C.; Smith, B. C.; Hill, R. B. Development and Validation of 2D Difference Intensity Analysis for Chemical Library Screening by Protein-Detected NMR Spectroscopy. Chembiochem 2018, 19, 448-458.
22. Congreve, M.; Carr, R.; Murray, C.; Jhoti, H. A ‘Rule of Three’ for fragment-based lead discovery? Drug Discovery Today 2003, 8, 876-877.
23. Schönherr, H.; Cernak, T. Profound Methyl Effects in Drug Discovery and a Call for New C□H Methylation Reactions. Angewandte Chemie International Edition 2013, 52, 12256-12267.
24. Hardegger, L. A.; Kuhn, B.; Spinnler, B.; Anselm, L.; Ecabert, R.; Stihle, M.; Gsell, B.; Thoma, R.; Diez, J.; Benz, J.; Plancher, J. M.; Hartmann, G.; Banner, D. W.; Haap, W.; Diederich, F. Systematic Investigation of Halogen Bonding in Protein-Ligand Interactions. Angewandte Chemie International Edition 2011, 50, 314-318.
25. Schick, S.; Rendeiro, A. F.; Runggatscher, K.; Ringler, A.; Boidol, B.; Hinkel, M.; Majek, P.; Vulliard, L.; Penz, T.; Parapatics, K.; Schmidl, C.; Menche, J.; Boehmelt, G.; Petronczki, M.; Muller, A. C.; Bock, C.; Kubicek, S. Systematic characterization of BAF mutations provides insights into intracomplex synthetic lethalities in human cancers. Nat Genet 2019, 51, 1399-1410.
26. Xiao, L.; Parolia, A.; Qiao, Y.; Bawa, P.; Eyunni, S.; Mannan, R.; Carson, S. E.; Chang, Y.; Wang, X.; Zhang, Y.; Vo, J. N.; Kregel, S.; Simko, S. A.; Delekta, A. D.; Jaber, M.; Zheng, H.; Apel, I J.; McMurry, L.; Su, F.; Wang, R.; Zelenka-Wang, S.; Sasmal, S.; Khare, L.; Mukherjee, S.; Abbineni, C.; Aithal, K.; Bhakta, M. S.; Ghurye, J.; Cao, X.; Navone, N. M.; Nesvizhskii, A. I.; Mehra, R.; Vaishampayan, U.; Blanchette, M.; Wang, Y.; Samajdar, S.; Ramachandra, M.; Chinnaiyan, A. M. Targeting SWI/SNF ATPases in enhancer-addicted prostate cancer. Nature 2022, 601, 434-439.
27. Alpsoy, A.; Utturkar, S. M.; Carter, B. C.; Dhiman, A.; Torregrosa-Allen, S. E.; Currie, M. P.; Elzey, B. D.; Dykhuizen, E. C. BRD9 Is a Critical Regulator of Androgen Receptor Signaling and Prostate Cancer Progression. Cancer Res 2021, 81, 820-833.
28. Bradford, M. M. A rapid and sensitive method for the quantitation of microgram quantities of protein utilizing the principle of protein-dye binding. Analytical Biochemistry 1976, 72, 248-254.
29. Delaglio, F.; Grzesiek, S.; Vuister, G.; Zhu, G.; Pfeifer, J.; Bax, A. NMRPipe: A multidimensional spectral processing system based on UNIX pipes. Journal of Biomolecular NMR 1995, 6.
30. Maciejewski, M. W.; Schuyler, A. D.; Gryk, M. R.; Moraru, I. I.; Romero, P. R.; Ulrich, E. L.; Eghbalnia, H. R.; Livny, M.; Delaglio, F.; Hoch, J. C. NMRbox: A Resource for Biomolecular NMR Computation. Biophysical Journal 2017, 112, 1529-1534.
31. Bartels, C. X., T.-H.; Billeter, M.; Guntert, P.; Wuthrich, K. The program XEASY for computer supported NMR Spectral Analysis of Biological Macromolecules. Journal of Biomolecular NMR 1995, 6 1-10.
32. Bahrami, A.; Assadi, A. H.; Markley, J. L.; Eghbalnia, H. R. Probabilistic interaction network of evidence algorithm and its application to complete labeling of peak lists from protein NMR spectroscopy. PLoS Comput Biol 2009, 5, e1000307.
33. Helmus, J. J.; Jaroniec, C. P. Nmrglue: an open source Python package for the analysis of multidimensional NMR data. J Biomol NMR 2013, 55, 355-67.
34. Pedregosa, F. V., G.; Gramfort, A.; Michel, V.; Thirion, B.; Grisel, 0.; Blondel, M.; Prettenhofer, P.; Weiss, R.; Dubourg, V.; Vanderplas, J.; Passos, A.; Coumapeau, D.; Brucher, M.; Perrot, M.; Duchesnay, E. Scikit-leam: machine learning in python. J. Mach. Learn. Res. 2011, 12.
35. Roos K, W. C., Damm W, Reboul M, Stevenson J M, Lu C, Dahlgren MK, Mondal S, Chen W, Wang L, Abel R, Friesner R A, Harder E D. OPLS3e: Extending Force Field Coverage for Drug-Like Small Molecules. Journal of Chemical Theory and Computation 2019, 15, 1863.
36. Greenwood, J. R.; Calkins, D.; Sullivan, A. P.; Shelley, J. C. Towards the comprehensive, rapid, and accurate prediction of the favorable tautomeric states of drug-like molecules in aqueous solution. Journal of Computer-Aided Molecular Design 2010, 24, 591-604.
37. Friesner, R. A.; Murphy, R. B.; Repasky, M. P.; Frye, L. L.; Greenwood, J. R.; Halgren, T. A.; Sanschagrin, P. C.; Mainz, D. T. Extra Precision Glide: Docking and Scoring Incorporating a Model of Hydrophobic Enclosure for Protein-Ligand Complexes. Journal of Medicinal Chemistry 2006, 49, 6177-6196.
38. Dutta, A.; Damarla, K.; Bordoloi, A.; Kumar, A.; Sarma, D. KOH/DMSO: A basic suspension for transition metal-free Tandem synthesis of 2,3-dihydroquinazolin-4(1H)-ones. Tetrahedron Letters 2019, 60, 1614-1619.
39. Sutherell, C.; Ley, S. On the Synthesis and Reactivity of 2,3-Dihydropyrrolo[1,2-a]quinazolin-5(1H)-ones. Synthesis 2016, 49, 135-144.
40. Jin, S.; Liu, Z.; Milbum, C.; Tomaszewski, M.; Walpole, C.; Wei, Z.-Y.; Yang, H. Preparation of benzamide derivatives for therapeutic use as cannabinoid receptor modulators. WO2005115972A1, 2005.
41. Dörner, B.; Kuntner, C.; Bankstahl, J. P.; Wanek, T.; Bankstahl, M.; Stanek, J.; Müllauer, J.; Bauer, F.; Mairinger, S.; Löscher, W.; Miller, D. W.; Chiba, P.; Mtiller, M.; Erker, T.; Langer, O. Radiosynthesis and in vivo evaluation of 1-[18F]fluoroelacridar as a positron emission tomography tracer for P-glycoprotein and breast cancer resistance protein. Bioorganic & Medicinal Chemistry 2011, 19, 2190-2198.

### Example 2: Supplementary Information of Example 1

TABLE 4
Fragment hits using protein-detected NMR
against the 2^{nd} bromodomain of PBRM1 (PBRM1-BD2).

| Compound | | |
|---|---|---|
| ID | Fragments | K_{d} (μM) |
| 5 | 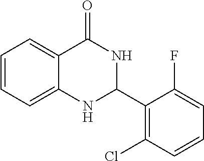 [SMILES: O=C1NC(c2c(F)cccc2Cl)Nc2ccccc21] | 45.3 ± 8.1 |
| 6 | 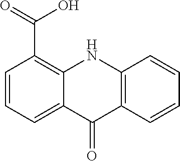 [SMILES: O=C(O)c1cccc2c(=O)c3ccccc3[nH]c12] | 79 ± 35 |
| 6a | 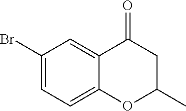 | 170 ± 35 |
| 6b | 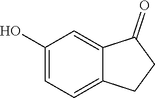 [SMILES: O=C1CCc2ccc(O)cc21] | 902 ± 270 |
| 6c | 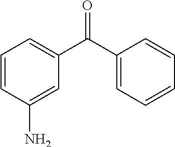 [SMILES: Nc1cccc(C(=O)c2ccccc2)c1] | 709 ± 211 |
| 6d | 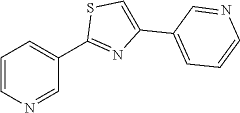 [SMILES: c1cncc(-c2csc(-c3cccnc3)n2)c1] | 1387 ± 262 |
| 6e | 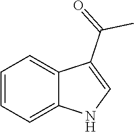 | 1195 ± 250 |
| 6f | 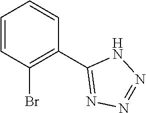 [SMILES: Brc1ccccc1-c1nnn[nH]1] | 1446 ± 599 |
| 6g | 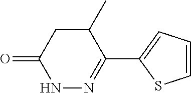 | 1210 ± 267 |
| 6h | 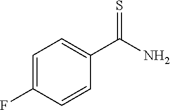 | >2000 |
| 6i | 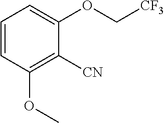 [SMILES: COc1cccc(OCC(F)(F)F)c1C#N] | >2000 |
| 6j | 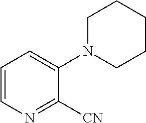 [SMILES: N#Cc1ncccc1N1CCCCC1] | >2000 |

TABLE 5
Comparison of the ability of our synthesized PBRM1-BD2 inhibitors (15, 16, 25, 26, and 34) vs. literature reported PBRM1-BD5 inhibitors (2 and
7) to stabilize bromodomains belonging to different bromodomain subfamilies as observed by differential scanning fluorimetry (DSF) assays.

| | | | | | |
|---|---|---|---|---|---|
| ΔTₘ (° C.)^{a} | <1 | 1-2 | 2-3 | 3-5 | >5 |

| | PBRM1- | PBRM1- | PBRM1- | PBRM1- | PBRM1- | PBRM1- | | | | | |
|---|---|---|---|---|---|---|---|---|---|---|---|
| Compound | BD1 | BD2^{a} | BD3 | BD4 | BD5 | BD6 | SMARCA2 | SMARCA4 | ASH1L | TRIM33A | BRD7 |
| 2 | −0.1 | 3.3 ± 0.4 | 2.6 | 0.3 | 7.7 | −1.4 | 2.2 | 2 | −0.7 | −0.9 | 1.2 |
| 7 | −0.9 | 7.7 ± 0.2 | 2.8 | 3.9 | 11.0 | −1.5 | 3.0 | 3.1 | −0.7 | 0.3 | 0.4 |
| 15 | −0.8 | 5.4 ± 0.1 | 2.7 | 0.9 | 2.9 | −2 | 0.5 | 0.4 | −0.6 | 0.8 | 0.2 |
| 16 | −0.1 | 5.4 ± 0.2 | 1.8 | 0.8 | 1.8 | −1.9 | 0.4 | 0.3 | −0.6 | 0.9 | 0.0 |
| 25 | −0.3 | 3.9 ± 0.3 | 1.9 | 0.2 | 0.9 | −1.3 | 0.0 | −0.4 | −0.8 | 0.3 | 0.0 |
| 26 | −0.5 | 5.2 ± 0.3 | 5.0 | 1.0 | 4.4 | −1.2 | 0.5 | 0.6 | −0.8 | 0.5 | 0.1 |
| 34 | 1.4 | 6.4 ± 0.2 | 0.6 | 0.2 | 1.5 | −2.4 | 0.3 | −0.2 | −0.4 | 0.1 | −0.4 |

| Compound | CREBBP | p300 | BRD2-BD2 | BRD3-BD1 | BRD3-BD2 | BRD4-BD1 | BRD4-BD2 | BRDT-BD1 | CECR2 | PCAF |
|---|---|---|---|---|---|---|---|---|---|---|
| 2 | 0.1 | −0.6 | −0.2 | 0.1 | 0.5 | 0.1 | −0.6 | −0.1 | 2.0 | −0.2 |
| 7 | −0.7 | −0.5 | 0.6 | −0.3 | 0.4 | −0.2 | −1.1 | −0.8 | −0.9 | 0.4 |
| 15 | 0.5 | 0.2 | 0.6 | 0.3 | 0.4 | 0.1 | −3.3 | −0.7 | −0.5 | 0.5 |
| 16 | 0.4 | −0.2 | 0.6 | 0.3 | 0.4 | 0.3 | −3.9 | −0.1 | −1.3 | 0.5 |
| 25 | 0.6 | 0.6 | 0.2 | 0.4 | 0.4 | 0.6 | −4.2 | 0.1 | 0.8 | 0.1 |
| 26 | 0.2 | 0.2 | 0.5 | 0.0 | 0.3 | 0 | −4.4 | −0.6 | 1.3 | 0.6 |
| 34 | −0.2 | −0.1 | 0.5 | 0.4 | 0.5 | 0.3 | −1.1 | −0.4 | −0.6 | 0.6 |

^{a}Values shown are the average of triplicates.

TABLE 6
Binding of the analogs of the second-best hit (6) to PBRM1-BD2
as observed by DSF assay and AlphaScreen

| | | PBRM1- | |
|---|---|---|---|
| | | BD2 | IC₅₀ |
| | | ΔTₘ | values |
| Compound | Structure | (º C.)^{a} | (μM) |
| 6 | 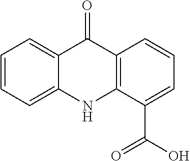 [SMILES: O=C(O)c1cccc2c(=O)c3ccccc3[nH]c12] | 1.5 ± 1.0 | 127 ± 30ª |
| 35 | 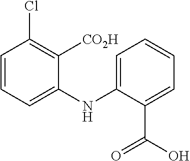 [SMILES: O=C(O)c1ccccc1Nc1cccc(Cl)c1C(=O)O] | 1.4 ± 0.2 | 26^{b} |
| 36 | 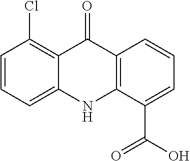 [SMILES: O=C(O)c1cccc2c(=O)c3c(Cl)cccc3[nH]c12] | 3.0 ± 0.1 | 7^{b} |
| 37 | 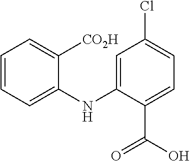 [SMILES: O=C(O)c1ccccc1Nc1cc(Cl)ccc1C(=O)O] | 0.3 ± 0.02 | >250^{b} |
| 38 | 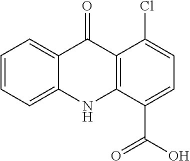 [SMILES: O=C(O)c1ccc(Cl)c2c(=O)c3ccccc3[nH]c12] | 0.0 | >250^{b} |

^{a}Values shown are the average of triplicate.
^{b}Values shown are single replicate.

Protein Purification

Expression and purification of recombinant ¹⁵N- and ¹³C/¹⁵N-labeled PBRM1-BD2 protein. Recombinant ¹⁵N-labeled PBRM1-BD2, and ¹³C/¹⁵N-labeled PBRM1-BD2 (pNIC28-Bsa4) were transformed into and purified from BL21(DE3) E. coli cells using nickel-affinity chromatography. ¹⁵N-labeled PBRM1-BD2 was grown at 37° C. in minimal media which contained 2 Medium P salts (50 mL, 10× stock: 500 mM Na₂HPO₄, 500 mM Na₂SO₄, 30 g/L ¹⁵N-labeled NH₄Cl, 5 g/L NaCl), 40 mL of 20% (w/v) glucose (sterile filtered), 2 mL vitamin solution (1 centrum vitamin dissolved in 20 mL of 50% (v/v) EtOH in ddH₂O), and 120 μL of 250 mM CaCl₂ (sterile filtered) per 1 L of media. ¹³C/¹⁵N-labeled PBRM1-BD2 was grown at 37° C. in minimal media supplemented with 2 g of ¹³C-glucose (instead of natural abundance glucose) per 1 L of media. All cultures were supplemented with 50 mg/L kanamycin and grown to an OD of ˜0.5-0.7 at 600 nm. Protein expression was induced overnight with 0.1 mM IPTG at 18° C. Cells were harvested via centrifugation at 5,000×g, and cell pellets were frozen at −80° C. until lysis. Frozen cells were thawed on ice and resuspended in lysis buffer (50 mM HEPES pH 7.5 at 20° C., 500 mM NaCl, 5% v/v glycerol, 5 mM Imidazole). Resuspended cells were immediately lysed via sonication, and lysates were cleared by centrifugation for 30 min at 30,000×g. Cleared lysates were then applied to Ni-NTA resin (0.75 mL resin/L culture) at 4° C. for at least 1 h while rocking. The protein-bound Ni-NTA resin was applied to a column and washed twice with 15 mL of lysis buffer and eluted using increasing concentrations of imidazole in lysis buffer (5 mL each of 50, 100, 150, 200, and 250 mM imidazole). Fractions were resolved by SDS-PAGE, and those containing PBRM1-BD2 were pooled and the N-terminal His₆ tag was removed using tobacco etch virus (TEV) protease (S2). Protein sample was re-applied to the Ni-NTA column and the flow-through was concentrated to a volume of 1 mL and applied to an Enrich SEC 70 10×300 mm column (Bio-Rad) into a storage buffer (100 mM Na₂PO₄ pH 6.5, 100 mM NaCl, and 1 mM DTT). Concentrations of purified proteins were determined by the method of Bradford using BSA as a standard,¹ aliquoted, flash-frozen, and stored at −80° C.

Expression and purification of recombinant bromodomains. Recombinant His 6-tagged PBRM1-BD1, PBRM1-BD2, PBRM1-BD3, PBRM1-BD4, PBRM1-BD5, PBRM1-BD6, SMARCA2, SMARCA4, ASH1L, TRIM33A, BRD7, CREBP, p300, BRD2-BD2, BRD3-BD1, BRD3-BD2, BRD4-BD1, BRD4-BD2, BRDT-BD1, CECR2, and PCAF were purified from BL21(DE3) E. coli cells using nickel affinity chromatography. BL21(DE3) cells were transformed and grown at 37° C. in TB media with 50 mg/L kanamycin to an OD of ˜0.6 at 600 nm. Protein expression was induced overnight with 0.1 mM IPTG at 18° C. Cells were harvested via centrifugation at 5,000×g and cell pellets were frozen at −80° C. until lysis. Frozen cells were thawed on ice and resuspended in lysis buffer (50 mM HEPES pH 7.5 at 20° C., 500 mM NaCl, 5% v/v glycerol, 5 mM Imidazole). Cells were lysed via sonication, and lysates were cleared by centrifugation for 30 min at 30,000×g. Cleared lysates were then applied to Ni-NTA resin (1 mL resin/L culture) at 4° C. for a minimum of 1 h while rocking. The supernatant was discarded, and the Ni-NTA resin was applied to a column and washed twice with 15 mL of lysis buffer. The protein was eluted using increasing concentrations of imidazole in lysis buffer (5 mL of 50, 100, 150, 200, and 250 mM imidazole). Fractions were monitored by SDS-PAGE, and those containing recombinant bromodomain were pooled. Protein samples were further purified and exchanged into a storage buffer (50 mM HEPES pH 7.5 at 20° C., 500 mM NaCl and 5% v/v glycerol) via size exclusion chromatography using an Enrich SEC 70 10×300 mm column (Bio-Rad). Concentrations of purified proteins were determined by the method of Bradford using BSA as a standard,¹ aliquoted, flash-frozen, and stored at −80° C.

Protein-Detected NMR Based Fragment Screening

Library selection. The Maybridge Library (Maybridge Ltd, Thermo Fisher Scientific, UK) and Zenobia Therapeutics Library 1 (Zenobia Therapeutics, San Diego, CA) contain 1000 and 968 fragments, respectively, with an average molecular weight of 154 Da, ranging from 94-286 Da. Fragments were received in 96-well plates and solubilized to 200 mM in DMSO-d₆. All fragments were soluble to 200 mM in DMSO and 1 mM in H₂O. For both libraries, each compound (10 μL; 1000 or 968 each in 200 mM DMSO-d₆) was mixed into 84 or 81 wells of a 96-well v-bottom plate, with each well containing twelve fragments at 16.67 mM each. An 85^{th} or 82^{th} well contained DMSO only. This step was performed manually resulting in a mother matrix plate with 85 or 82 wells containing a total of 120 μL of fragments (or DMSO) in DMSO-d₆. Then, 2.1 μL of each fragment mixture (or DMSO) from the mother matrix plate was transferred to single-use daughter plate 96-well v-bottom plates. Mother and daughter plates were sealed and stored at −80° C. Chemicals for subsequent NMR titration experiments were purchased from Enamine, AK Scientific, or Sigma-Aldrich. For individual titration experiments, compounds were initially solubilized to 200 mM in DMSO-d₆ and stored at −80° C. until further use.

Fragment screening and semi-automated preparation of NMR samples.² For NMR-based screening, 50 μM ¹⁵N-labeled PBRM1-BD2 in 100 mM Na₂HPO₄, 100 mM NaCl, 0.02% NaN₃, pH 6.5 with 8% (v/v) D₂O was used. In each well, ¹⁵N-labeled protein (67.9 μL, 50 μM) was added to fragment mixture (2.1 μL) to give final concentrations of 3% (v/v) DMSO-d₆, 500 μM of each fragment, and 48.5 μM protein. Subsequent steps used a PAL liquid handling robot (LEAP Technologies), configured in-house for solution mixing and loading of 1.7 mm diameter NMR sample tubes. To avoid cross-contamination, the syringe was washed with acetonitrile and water between samples. Each NMR sample was mixed and transferred from the 96-well v-bottomed plate to 1.7 mm NMR tubes. Upon completion of sample preparation, NMR tubes were capped, and any signs of precipitation/aggregation were noted. Total time spent for sample preparation for NMR screening was ˜4 h, or 2.5 min/sample.

Primary screening identified a subset of NMR samples containing fragment(s) with affinity to the protein target. To identify these, twelve fragments from the hit sample were divided into four groups each consisting of three fragments, and 2.1 μL of each sample was aliquoted into a 96-well v-bottom plate. Then, ¹⁵N-labeled protein (67.9 μL, 50 μM) was added, and the solution was gently mixed and transferred into 1.7 mm NMR tubes by using the PAL liquid-handling robot to give final concentrations of 3% (v/v) DMSO-d₆, 500 μM fragment, and 48.5 μM protein.

The three fragments from the samples that exhibited an affinity for the protein were further aliquoted individually into three wells of a 96-well v-bottom plate. Then, ¹⁵N-labeled protein (67.9 μL, 50 μM) was added, and the solution was gently mixed and transferred into 1.7 mm NMR tubes by using the PAL liquid-handling robot to give final concentrations of 3% (v/v) DMSO-d₆, 500 μM fragment, and 48.5 μM protein. To determine binding affinities of fragments against protein targets, titration experiments by NMR were performed as previously described.² Briefly, the PAL robot was used to set up one DMSO control (3% v/v) and eight titration points per fragment at 47 μM, 94 μM, 188 μM, 375 μM, 750 μM, 1500 μM, 3000 μM, 6000 μM with ¹⁵N-labeled protein (48.5 μM).

NMR spectroscopy. Automated NMR data collection was performed on ¹⁵N-labeled PBRM1-BD2 (50 μM) by using ¹H,¹⁵N SOFAST-HMQC experiments. Data was collected at 25° C. on a Bruker Avance II 600 MHz spectrometer equipped with a triple resonance z-axis gradient cryoprobe and SampleJet autosampler, which allowed automatic tuning, matching, and shimming for each sample. ¹H,¹⁵N SOFAST-HMQC experiments consisted of 32 scans with 1024 and 210 complex points in the ¹H and ¹⁵N dimensions, respectively. The total time for screening 85 or 82 samples each containing 12 fragments or DMSO by ¹H,¹⁵N SOFAST-HMQC was 46 h (˜32 min/sample). NMR samples of ¹⁵N-PBRM1-BD2 (50 μM) contained Na₂HPO₄ (100 mM) pH 6.5, NaCl (100 mM), with 8% v/v D₂O. Resonance assignments for PBRM1-BD2 were partially obtained from published values (PDB ID: 2KTB).³ In addition, ¹⁵N HSQC, HNCA, HNCACB, HNCO, HN(CO)CA, and HN(CO)CACB experiments were used to confirm assignments and distinguish between overlapping peaks in the ¹H, ¹⁵N HSQC spectrum. All NMR data were processed using NMRPipe⁴ in NMRbox.⁵ Spectra were processed by using in-house automated Python scripts in NMRPipe,⁴ and chemical shifts perturbation were analyzed using SPARKY.⁶ Total ¹H/¹⁵N chemical shift perturbations and equilibrium dissociation constants (K_{d}) were calculated as reported.²

Principal component analysis (PCA) and K-means clustering.²¹ 2D NMR spectra of 1024 and 512 complex points in proton and nitrogen dimensions, respectively, were used as input data for PCA.²⁹ The spectral regions 6-10 ppm ¹H and 105-135 ppm ¹⁵N were included for analysis. The first 3 principles components were used as input for K-means clustering. PCA and K-means clustering analyses were carried out using the Scikit-learn python package.³⁴

Difference intensity analysis (DIA).² By using the addNMR module (or in-house Python scripts) of NMRPipe,⁴ 2D difference spectra were generated from heteronuclear data from each fragment mixture compared to the DMSO control. Total cross peak intensities were calculated from 2D difference spectra in NMRPipe using in-house Python scripts. Only crosspeak intensities >300000 were included. The resulting 2D difference spectra for each fragment mixture were condensed into a single positive (and negative) value representing the total sum of all positive (and negative) crosspeak intensities. Accordingly, each NMR sample gave two values. NMR samples containing fragments that gave rise to total (positive and negative) crosspeak intensity value(s) greater than one standard deviation from the mean across all 1000 samples were considered as either potential binders (+1σ and −1σ) or compounds that induce nonspecific NMR crosspeak broadening (−1σ only).

Computational fragment docking. Crystal structures of PBRM1-BD2 in apo conformation (PDB ID: 3LJW) or bound to inhibitor compounds (PDB ID: 6ZN6 and 6ZNV) were obtained from the protein databank and imported into the Schrödinger (Release 2017-3) Maestro suite using the Protein Preparation wizard and subjected to restrained minimization within the OPLS3e force field.⁷ Fragment structures were prepared for docking using the LigPrep application in Maestro, and ligand ionization states at pH 7.0±2.0 were generated using Epik.⁸ Fragments were flexibly docked into a 30×30×30 Å grid centered on the centroid of the minimized PBRM1-BD2 structure coordinates using Glide in extra-precision (XP) mode.⁹ Output poses were ranked according to the Emodel score.

AlphaScreen. AlphaScreen beads and 96-well ½-area light gray plates (Part number: 6002350) were purchased from PerkinElmer. A biotinylated H3K14ac peptide (NH₂-ARTKQTARKSTGGK(Ac)APRKQLK(biotin)-CONH₂) was purchased from Peptide 2.0. All reagents were diluted in Epigenetic Buffer (5×), 2 μM TCEP, and 0.5% v/v Tween-20. The assay used a final volume of 40 μL. Stock solutions of inhibitors in DMSO were made at eight different concentrations (100 mM, 10 mM, 1 mM, 300 μM, 100 μM, 30 μM, 10 μM, 3 μM). 0.4 μL of inhibitor DMSO stocks were further diluted to 10 μL in the buffer. To a 17.5 μL mixture consisting of 400 nM of His₆-tagged PBRM1-BD2, 200 nM of the biotinylated H3K14ac peptide, 2.5 μL of 0-4 mM of inhibitors (0.25% v/v final DMSO) were titrated and the mixture (20 μL) was incubated for 30 min. 20 μL of AlphaScreen beads (20 μg/mL Streptavidin Donor beads, 5 μg/mL Nickel-chelate Acceptor beads) were added to achieve the final concentration of 200 nM His-tagged protein, 100 nM biotinylated H3K14ac, 0-250 μM of inhibitors. The mixture (40 μL) was further incubated for 1 h in a dark room and the luminescence was read on a BioTek Cytation5 Imaging Reader using an AlphaScreen detection filter cube. Data was plotted in GraphPad Prism using non-linear regression dose-response three variable slopes.

$$y = {y}_{min} + \frac{{y}_{max} - {y}_{min}}{1 + 1 {0}^{( log ( conc . ) - log ( {IC}_{50} ) )}}$$

$$y = Normalized AlphaScreen count ;$$

$${y}_{max} = Maximum AlphaScreen count ;$$

$${y}_{min} = Maximum AlphaScreen count$$

Differential Scanning Fluorimetry (DSF). Bromodomain DSF Tₘ assays were performed using an Mx3005P (Stratagene) PCR detection system and low profile 96-well PCR plates (UltraFlux, Flat Top, SSI 3400-00S). PBRM1-BD2 was buffered in 100 mM Na₂HPO₄, 500 mM NaCl, pH 6.5 and assayed at a concentration of 10 μM with 100 μM inhibitor and 5× SyproOrange (Invitrogen). Excitation and emission filters were set to 492 and 610 nm respectively. The temperature was raised by 1° C./min from 25-95° C., and fluorescence readings were taken at each interval. Melting curves were analyzed using our online interactive fitting tool for thermofluor experiments (https://dsf-analysis.herokuapp.com/DSF-analysis) and plotted using GraphPad Prism.

Isothermal Titration Calorimetry. Binding affinities of inhibitors towards PBRM1-BD2, PBRM1-BD5, SMARCA2, SMARCA4, and ASH1L were determined using a VP-ITC instrument (MicroCal). For each inhibitor, 210 μM PBRM1-BD2, PBRM1-BD5, SMARCA2, SMARCA4, or ASH1L was injected (1×2 μL injection followed by 30×10 μL injections) into the cell containing 30 μM inhibitor, and heats of binding were measured. Protein concentrations were determined via the method of Bradford.¹ The buffer used for all ITC experiments consisted of 50 mM HEPES (pH 7.5 at 20° C.), 500 mM NaCl, 5% v/v glycerol, and 1% v/v DMSO; except for experiments involving PBRM1-BD2 whose buffer consisted of 100 mM Na₂PO₄ (pH 7.5 at 20° C.), 100 mM NaCl, 5% v/v glycerol, and 1% v/v DMSO. K_{d} values were determined by least-squares fitting to the raw data using Origin (OriginLab).

Cell Culture Conditions. LNCaP (clone FGC) (RRID:CVCL_1379), PC3 (RRID:CVCL_0035), RWPE-1 (RRID:CVCL_3791), HEK293T (RRID:CVCL_0063), cells were purchased from American Type Culture Collection (ATCC, Manassas, VA). HEK293T cells were cultured in DMEM media supplemented with 10% fetal bovine serum, 100 units/ml penicillin and 100 g/ml streptomycin, and 2 mM L-alanyl-L-glutamine (Corning Glutagro™) and 1:10000 plasmocin (Invivogen, San Diego, CA). LNCaP cells were cultured in RPMI 1640 and PC3 cells were cultured in F12K media with the same supplements as above. RWPE-1 cells were cultured in Keratinocyte SFM (Gibco 17005-042, Thermo Scientific). All other media and supplements were obtained from Corning Mediatech, Inc. All cultures were used up to passage number 20 and tested monthly for Mycoplasma contamination with MycoAlert™ Mycoplasma Detection Kit (Lonza, Switzerland).

Lentivirus. HEK293T cells were transfected with lentivirus constructs along with packaging vectors pMD2.G and psPAX2. After 48 h, the supernatant was collected and concentrated by ultracentrifugation (17,300 rpm for 2 h) and resuspended in 200 μL of PBS. PBRM1 knockdown was performed by transfection with pLKO.¹ containing shRNA against PBRM1 (TRCN0000015994, Thermo Fisher Scientific) and control vector pLKO.¹ containing scrambled shRNA.

PBRM1 knockdown and viability measurement. 500,000-600,000 cells were seeded in 6-cm plates. When cells reached 50-80% confluency, (24-72 hours) lentivirus containing pLKO.¹ empty vector or shPBRM1, such that the cells were before transduction. 24 hours after transduction, the cells were selected by treatment with puromycin (2 μg/mL) for 48 hours. The cells were then counted and seeded in a 96-well plate in a density of 5000 cells per well, and grown for 4 days. Cell viability was measured with a CellTiter-Glo® kit at day 0 and day 4 and calculated as a percentage of luminescence.

Nuclear Lysate Preparation. Cells (0.5×10⁶) were lysed in 500 μL of Buffer A (20 mM HEPES, pH 7.9, 25 mM KCl, 10% glycerol, 0.1% Nonidet P-40 with protease inhibitors for 10 min on ice. Nuclei were pelleted at 600×g for 10 min and resuspended in lysis buffer (20 mM HEPES, pH 7.5, 200 mM KOAc, 0.2% NP-40, 2 mM MgCl₂) containing protease inhibitors. Nuclei were rotated for 30 min at 4° C., and the lysate was cleared at 21,000×g for 10 min.

Immunoblot analysis. Nuclear lysates were quantified using BCA Assay (Pierce, Rockford, IL). Equal amounts of protein were electrophoresed on Novex 4-12% precast gel, transferred onto 0.45 μm PVDF membrane, and blocked 1h in 5% BSA in TBST. After overnight incubation in primary antibodies, the membrane was incubated with infrared-dye labeled Licor goat anti-mouse or anti-rabbit antibodies. The images were acquired using Li-Cor® imaging system.

Cell Viability Assays. Cells were seeded in a 96-well plate and incubated for 12-24 h prior to initial treatment. The number of cells per well was chosen empirically based on cell growth rates (LNCaP-5,000 cells/well, PC3 and RWPE-1-3,000 cells per well, HEK293T-1,000 cells/well). Cells were treated with either DMSO control or serial dilutions of inhibitors and media with compound was refreshed every 48 h. Cells were harvested when control samples reached confluence (293T cells-5 days, PC3 cells-6 days, and RWPE-1 and LNCaP cells −7 days) and cell viability was measured via CellTiter-Glo® (Promega).

Statistics. Statistics were performed using GraphPad Prism 9. Statistical details are provided in figure legends and within the text.

Peptide Pulldown. LNCaP cells (10×10⁶) were harvested and lysed in 5 mL of Buffer A (25 mM Hepes, pH 7.6, 25 mM KCl, 5 mM MgCl₂, 0.05 mM EDTA, 0.1% Nonidet P-40, 10% glycerol, protease inhibitors) for 10 min on ice. Nuclei were pelleted at 600×g for 10 min and resuspended in 2 mL of Binding Buffer 1 (25 mM Tris, pH 8, 250 mM NaCl, 1% NonidetP-40, 1 mM EDTA, plus protease inhibitors) for multiply acetylated peptide binding analysis or Binding Buffer 2 (25 mM Tris, pH 8, 150 mM NaCl, 1% Nonidet P-40, 1 mM EDTA, plus protease inhibitors) for singly acetylated peptide binding analysis, and rotated at 4° C. for 10 min. The lysate was cleared at 21,000×g, for 10 min. The lysate was split and 50 μM inhibitor was added to one half while an equal volume of DMSO was added to the other half and both tubes were incubated 10 min on ice. Lysates (200 μL) was added to 2 μg of biotin-labeled peptide (AnaSpec), and samples were rotated at 4° C. for 1 h. The following peptides were used: H₃(1-30), H3K14Ac(1-30), and H3K14/18/23/27Ac(1-30). Streptavidin Agarose Ultra Performance Resin (15 μL) (Solulink, San Diego, CA) was washed three times with Binding Buffer and added to lysates for 30 min to capture peptides. The beads were washed for 5 min three times in their appropriate Binding Buffer and resuspended in 1× Bolt LDS Sample Buffer (Invitrogen) and boiled for 5 min. Nuclear lysate input and the samples were processed for immunoblotting analysis.

### Synthesis

General experimental. Reagents were from Sigma-Aldrich, AA Blocks, TCI, Ambeed Inc. unless otherwise stated. Anhydrous solvents used in reactions were either analytical grade as obtained commercially (Sigma-Aldrich) or were dried via Grubbs apparatus. HPLC grade solvents were employed for work-up and chromatography. Reagents were used as supplied (analytical or HPLC grade) without prior purification. Anhydrous MgSO₄ was used as a drying agent.

Thin-layer chromatography was performed using glass plates coated with 60 F254 silica. Plates were visualized using UV light (254 nm) or 1% (w/v) aq. KMnO4 stain. MPLC was performed using Biotage Sfar HC-Duo or SNAP Ultra columns on Biotage Isolera in series with a Biotage Dalton 2000 mass spectrometer. Retention factors (R_{f}) are quoted to a precision of 0.05.

Microwave reactions were carried out using a Biotage Initiator+.

Deuterated solvents were from Sigma. ¹H NMR and ¹³C NMR spectra were recorded using Bruker AVIII500 NMR spectrometer equipped with a TCI cryoprobe. Fields were locked by external referencing to the relevant residual deuterium resonance. Chemical shifts (6) are reported in ppm; coupling constants (J) are recorded in Hz to the nearest 0.5 Hz; when peak multiplicities are reported, the following abbreviations are used: s=singlet, d=doublet, t=triplet, q=quartet, m=multiplet, br=broadened, dd=doublet of doublets, dt=doublet of triplets, td=triplet of doublets. Spectra were recorded at room temperature unless otherwise stated.

Low-resolution mass spectra (m/z) were recorded using Biotage Dalton 2000 by using either an APCI or ESI probe. High-resolution mass spectra were recorded at the Indiana University Mass Spectrometry Facility on a Thermo Scientific Orbitrap XL spectrometer by use of the indicated ionization method. A post-acquisition gain correction was applied using reserpine as a lock mass.

Purities of compounds were >95% as assessed by analytical high performance liquid chromatography (HPLC) for key compounds unless otherwise stated. Analytical HPLC was carried out on a Hypersil GOLD column (C18, 250′ 4.6 mm) using a flow rate of 1 mL/min with detection at 235 and/or 256 nm. The method was as follows using H₂O with 0.1% v/v trifluoroacetic acid as eluent A and MeOH with 0.1% v/v trifluoroacetic acid as eluent B.

| Time (min) | A (%) | B (%) |
|---|---|---|
| 0 | 95 | 5 |
| 30 | 5 | 95 |
| 35 | 5 | 95 |
| 36 | 95 | 5 |
| 41 | 95 | 5 |

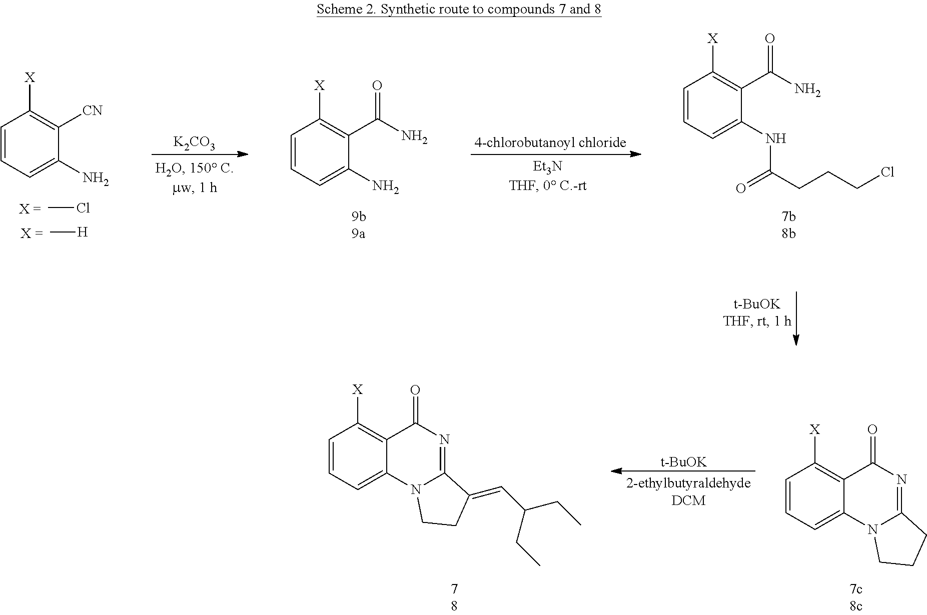

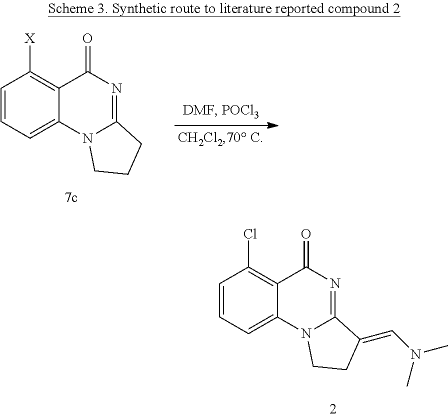

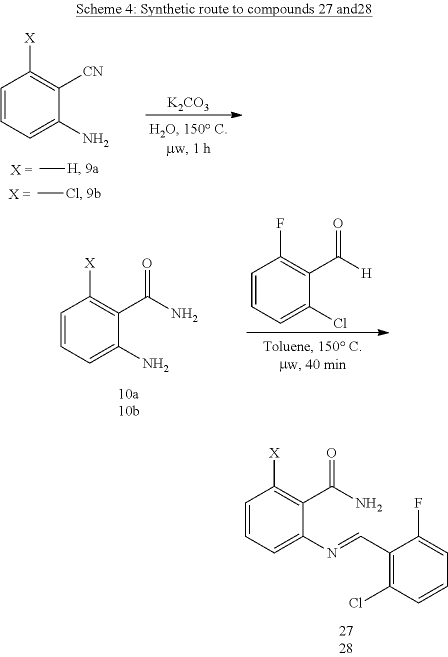

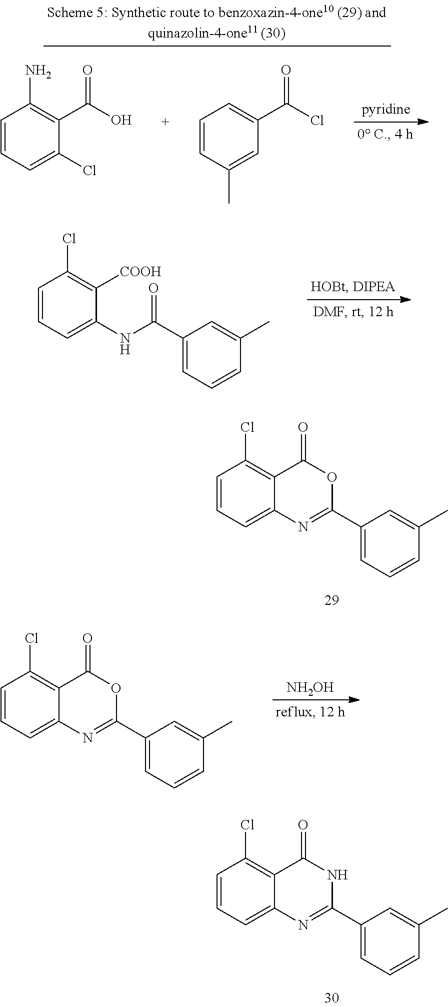

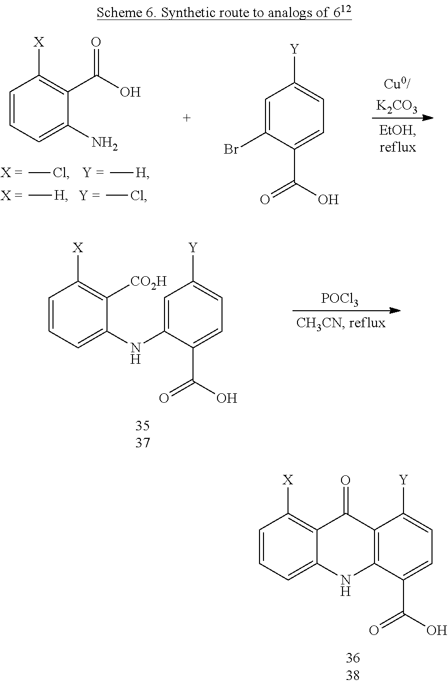

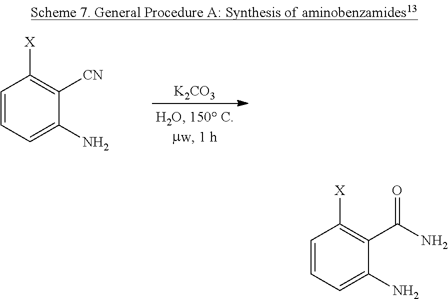

The desired compound was obtained according to the reported procedure. The appropriate aminobenzonitrile (1 mmol) and K₂CO₃ (0.2 mmol) were added to deionized water (8.5 mL/mmol substrate) in a 20 mL microwave vial charged with a stir bar. The reaction was irradiated under microwave at 150° C. for 1.5 h. After the consumption of the starting material (as assessed by TLC), the reaction was cooled to room temperature and then extracted with ethyl acetate (3×10 nL). The combined organic layer was dried (anhydrous MgSO₄) and concentrated under vacuum. The resulted residue was purified via flash chromatography.

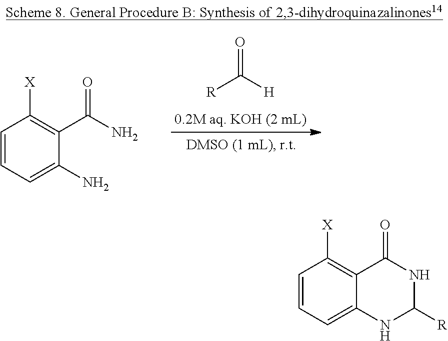

To a stirred solution of appropriate aminobenzamide (1 mmol) in DMSO (1 mL/mmol) and 0.2 M KOH (2 mL/mmol) was added appropriate benzaldehyde (1.2 mmol) dropwise and the mixture was heated to the required temperature (rt to 80° C.). After the consumption of the starting material (as assessed by TLC), the reaction was cooled to room temperature, and the crude product mixture was concentrated under reduced pressure, then diluted with ethyl acetate and washed with water (3×10 mL). The organic layer was dried (anhydrous MgSO₄) and concentrated under vacuum. The resulted residue was purified via flash chromatography.

### Compound Characterization

### 2-Aminobenzamide (9a)

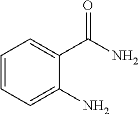

2-Aminobenzonitrile (1.4 g, 12.7 mmol) was hydrolyzed according to general procedure A. Purification by MPLC (1% EtOAc/hexanes to 60% EtOAc/hexanes) gave a white solid (1.4 g, 88%). TLC R_{f} 0.35 (50% EtOAc/hexanes); ¹H NMR (500 MHz, DMSO-d₆) δ 7.68 (s, 1H), 7.49 (d, J=8.0 Hz, 1H), 7.10 (t, J=8.0 Hz, 1H), 7.02 (s, 1H), 6.64 (d, J=8.0 Hz, 1H), 6.53 (s, 2H), 6.45 (t, J=7.5 Hz, 1H); ¹³C NMR (126 MHz, DMSO-d₆) δ 171.2, 150.1, 131.8, 128.7, 116.3, 114.3, 113.6; LRMS (APCI) m/z: Calculated for C₇H₁₀N₂O [M+H]⁺ 137.1, Observed: 137.1. Analytical data are consistent with those reported.¹³

### 2-Amino-6-chlorobenzamide (9b)

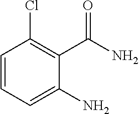 [SMILES: NC(=O)c1c(N)cccc1Cl]

2-Amino-6-chlorobenzonitrile (2 g, 13.2 mmol) was hydrolyzed according to general procedure A. Purification by MPLC (1% EtOAc/hexanes to 80% EtOAc/hexanes) gave a white solid (1.8 g, 81%). TLC R_{f} 0.65 (50% EtOAc/hexanes); ¹H NMR (500 MHz, DMSO-d₆) δ 7.79 (s, 1H), 7.56 (s, 1H), 6.99 (t, J=8.0 Hz, 1H), 6.61 (d, J=8.0 Hz, 1H), 6.56 (d, J=8.0 Hz, 1H), 5.19 (s, 2H); ¹³C NMR (126 MHz, DMSO-d₆) δ 167.5, 147.0, 129.9, 129.8, 121.6, 116.1, 113.6; LRMS (APCI) m/z: Calculated for C₇H₈³⁵ClN₂O [M+H]⁺ 171.0, Observed: 171.0. Analytical data are consistent with those reported.¹³

### 2-Amino-6-methoxybenzamide (9c)

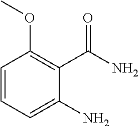

2-Amino-6-methoxybenzonitrile (740 mg, 5 mmol) was hydrolyzed according to general procedure A. Purification by MPLC (1% EtOAc/hexanes to 80% EtOAc/hexanes) gave white solid (720 mg, 87%). TLC R_{f} 0.2 (50% EtOAc/hexanes); ¹H NMR (500 MHz, DMSO-d₆) δ 7.51 (s, 1H), 7.24 (s, 1H), 6.99 (t, J=8.0 Hz, 1H), 6.33 (s, 2H), 6.28 (d, J=8.0 Hz, 1H), 6.15 (d, J=8.0 Hz, 1H), 3.74 (s, 3H); ¹³C NMR (126 MHz, DMSO-d₆) δ 169.2, 158.4, 151.0, 131.0, 109.3, 105.1, 97.9, 55.5; LRMS (APCI) m/z: Calculated for C₈H₁₁N₂O₂ [M+H]⁺ 167.1, Observed: 167.1. Analytical data are consistent with those reported.¹³

### 2-Amino-6-methylbenzamide (9d)

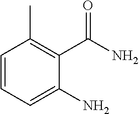

2-Amino-6-methylbenzonitrile (132 mg, 1 mmol) was hydrolyzed according to general procedure A. Purification by MPLC (1% EtOAc/hexanes to 40% EtOAc/hexanes) gave a white solid (133 mg, 89%). TLC R_{f} 0.7 (50% EtOAc/hexanes); ¹H NMR (500 MHz, DMSO-d₆) δ 7.61 (s, 1H), 7.40 (s, 1H), 6.90 (t, J=8.0 Hz, 1H), 6.49 (d, J=8.0 Hz, 1H), 6.37 (d, J=7.5 Hz, 1H), 4.88 (s, 2H), 2.19 (s, 3H); ¹³C NMR (126 MHz, DMSO-d₆) δ 171.0, 145.9, 134.7, 129.2, 123.5, 118.4, 113.2, 20.4; LRMS (APCI) m/z: Calculated for C₈H₁₁N₂O [M+H]⁺ 151.1, Observed: 151.1. Analytical data are consistent with those reported.¹⁵

### 2-Amino-6-bromobenzamide (9e)

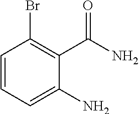 [SMILES: NC(=O)c1c(N)cccc1Br]

2-Amino-6-bromobenzonitrile (500 mg, 2.6 mmol) was hydrolyzed according to general procedure A. Purification by MPLC (1% EtOAc/hexanes to 100% EtOAc/hexanes) gave a white solid (480 mg, 87%). TLC R_{f} 0.1 (50% EtOAc/hexanes); ¹H NMR (500 MHz, DMSO-d₆) δ 7.79 (s, 1H), 7.53 (s, 1H), 6.92 (t, J=8.0 Hz, 1H), 6.71 (d, J=8.0 Hz, 1H), 6.64 (d, J=8.0 Hz, 1H), 5.13 (s, 2H); ¹³C NMR (126 MHz, DMSO-d₆) δ 168.3, 146.7, 144.5, 130.0, 124.0, 119.2, 114.0; LRMS (APCI) m/z: Calculated for C₇H₈⁷⁹BrN₂O [M+H]⁺ 215.0, Observed: 215.0. Analytical data are consistent with those reported.¹³

### 2-Chloro-6-(4-chlorobutanamido)benzamide (7b)

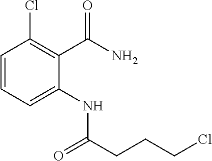 [SMILES: NC(=O)c1c(Cl)cccc1NC(=O)CCCCl]

The desired compound was obtained according to the reported procedure.¹³ To a stirred solution of 6-chloro-2-aminobenzamide (9b; 170 mg, 1 mmol) in THF (2.5 mL/mmol) at 0° C. were added triethylamine (0.3 mL, 2 mmol) and 4-chlorobutanoyl chloride (0.15 mL, 1.2 eq) in TIF (2 mL/mmol). After the completion of the reaction (as assessed by TLC), the reaction mixture was extracted with ethyl acetate and washed with water (3×10 mL). The combined organic layer was dried (anhydrous MgSO₄) and concentrated under vacuum. The residue was purified via MPLC (1% EtOAc/hexanes to 50% EtOAc/hexanes) resulting in a white amorphous solid (220 mg, 80%). TLC R_{f}0.40 (50% EtOAc/hexanes); ¹H NMR (500 MHz, DMSO-d₆) δ 9.32 (s, 1H), 7.88 (s, 1H), 7.72 (s, 1H), 7.59 (d, J=8.0 Hz, 1H), 7.33 (t, J=8.0 Hz, 1H), 7.26 (d, J=8.0 Hz, 1H), 3.66 (t, J=6.5 Hz, 2H), 2.45 (d, J=7.0 Hz, 2H), 1.98 (p, J=7.0 Hz, 2H); ¹³C NMR (126 MHz, DMSO-d₆) δ 174.4, 170.7, 166.0, 135.8, 129.7, 129.4, 125.5, 123.9, 44.7, 32.9, 28.1; LRMS (APCI) m/z: Calculated for C₁₁H₁₃³⁵Cl₂N₂O₂ [M+H]⁺ 275.0, Observed: 275.0. Analytical data are consistent with those reported.¹³

### 2-(4-Chlorobutanamido)benzamide (8b)

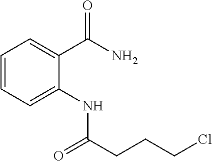 [SMILES: NC(=O)c1ccccc1NC(=O)CCCCl]

The desired compound was obtained according to the reported procedure.¹³ To a stirred solution of 2-aminobenzamide (1 g, 7.35 mmol) in THF (20 mL) at 0° C. were added triethylamine (2 mL, 14.7 mmol) and 4-chlorobutanoyl chloride (1 ml, 8.8 mmol) in THF (18 mL). After the completion of the reaction (as assessed by TLC), the reaction mixture was extracted with ethyl acetate and washed with water (3×10 mL). The combined organic layer was dried (anhydrous MgSO₄) and concentrated under vacuum. The residue was purified via MPLC (1% EtOAc/hexanes to 100% EtOAc/hexanes) which resulted in the title compound (1.4 g, 80%). TLC R_{f} 0.6 (5% MeOH/DCM); ¹H NMR (500 MHz, DMSO-d₆) δ 11.68 (s, 1H), 8.41 (d, J=8.0 Hz, 11H), 8.24 (s, 1H), 7.77 (dd, J=8.0, 1.5 Hz, 1H), 7.70 (s, 1H), 7.46 (td, J=8.0, 1.5 Hz, 1H), 7.09 (t, J: 7.5 Hz, 1H), 3.68 (t, J=6.5 Hz, 2H), 2.48 (t, J=7.5 Hz, 2H), 2.03 (p, J=7.0 Hz, 2H); ¹³ C NMR (126 MHz, DMSO-d₆) δ 170.7, 169.9, 139.4, 132.1, 128.5, 122.3, 120.1, 119.6, 44.6, 34.3, 27.8; LRMS (APCI) m/z: Calculated for C₁₁H₁₄³⁵ClN₂O₂[M+H]⁺ 241.1, Observed: 241.1. Analytical data are consistent with those reported.¹³

### 6-Chloro-2,3-dihydropyrrolo[1,2-a]quinazolin-5(1H)-one (7c)

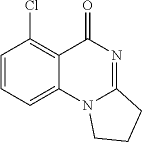 [SMILES: O=c1nc2n(c3cccc(Cl)c13)CCC2]

The desired compound was obtained according to the reported procedure. ¹³ To a stirred solution of 2-chloro-6-(4-chlorobutanamido)benzamide (7b; 180 mg, 0.7 mmol) in TIF (10 mL/mmol) was added t-BuOK (147 mg, 1.3 mmol). After the completion of the reaction (as assessed by TLC), the solvent was removed under reduced pressure. The crude mixture was diluted with CH₂Cl₂ and washed with Na—HCO₃ (15 mL), then water and finally with brine. The combined organic layer was dried (anhydrous MgSO₄) and concentrated under vacuum. Purification of the resulted residue via MPLC (1% MeOH/DCM to 5% MeOH/DCM) gave the title compound as a white amorphous solid (70 mg, 50%). TLC R_{f} 0.3 (5% MeOH/DCM); ¹H NMR (500 MHz, DMSO-d₆) δ 7.69 (t, J=8.0 Hz, 1H), 7.46 (d, J=8.0 Hz, 1H), 7.40 (d, J=8.5 Hz, 1H), 4.18 (t, J=7.5 Hz, 2H), 2.99 (t, J=8.0 Hz, 2H), 2.22 (p, J=7.5 Hz, 2H); 13C NMR (126 MHz, DMSO-d₆) δ 166.7, 166.0, 141.1, 133.7, 133.4, 127.8, 115.1, 114.8, 49.3, 32.0, 18.0; LRMS (APCI) m/z: Calculated for C₁₁H₁₀³⁵ClN₂O [M+H]⁺ 221.0, Observed: 221.0. Analytical data are consistent with those reported.¹³

### 2,3-Dihydropyrrolo[1,2-a]quinazolin-5(1H)-one (8c)

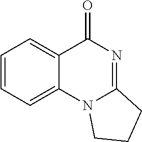 [SMILES: O=c1nc2n(c3ccccc13)CCC2]

The desired compound was obtained according to the reported procedure. 13 To a stirred solution of 6-(4-chlorobutanamido)benzamide (8b; 1.4 g, 5.8 mmol) in THF (30 mL) was added t-BuOK (1.3 g, 11.7 mmol). After the completion of the reaction (as assessed by TLC), the solvent was removed under reduced pressure. The crude mixture was diluted with CH₂Cl₂ and washed with NaHCO₃ (15 mL), then water, and finally with brine. The combined organic layer was dried (anhydrous MgSO₄) and concentrated under vacuum. The resulted residue was purified via MPLC (1% MeOH/DCM to 5% MeOH/DCM) which gave the title compound (0.6 g, 50%). TLC R_{f} 0.3 (5% MeOH/DCM); H NMR (500 MHz, DMSO-d₆) δ 8.03 (d, J=8.0 Hz, 1H), 7.78 (td, J=7.5, 1.5 Hz, 1H), 7.45 (dd, J=8.0, 7.5 Hz, 2H), 4.25-4.19 (m, 2H), 3.01 (t, J=8.0 Hz, 2H), 2.23 (p, J=8.0 Hz, 2H); ¹³C NMR (126 MHz, DMSO-d₆) δ 169.0, 166.8, 138.7, 133.6, 127.3, 125.3, 118.1, 115.8, 48.5, 32.3, 18.2; LRMS (APCI) m/z: Calculated for C₁₁H₁₁N₂O [M+H]⁺ 187.1; Observed: 187.1. Analytical data are consistent with those reported. ¹³

### (E)-6-Chloro-3-((dimethylamino)methylene)-2,3-dihydropyrrolo[1,2-a]quinazolin-5(1H)-one (2)

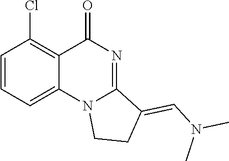 [SMILES: CN(C)/C=C1\CCn2c1nc(=O)c1c(Cl)cccc12]

The desired compound was obtained according to the reported procedure. 13 To a stirred solution of 6-chloro-2,3-dihydropyrrolo[1,2-a]quinazolin-5(1H)-one (7c; 54 mg, 0.24 mmol) in anhydrous DMF (2 mL) was added phosphoryl trichloride (0.5 mL, 0.5 mmol). The reaction was heated to 70° C. till the completion of the reaction (as assessed by TLC). The reaction was then cooled to room temperature and the solvent was removed under reduced pressure. The crude mixture was diluted with CH₂Cl₂ and washed with NaHCO₃ (15 mL), and the aqueous layer was extracted with CH₂Cl₂. The combined organic layer was dried (anhydrous MgSO₄) and concentrated under vacuum. The residue was purified via MPLC (1% MeOH/DCM to 5% MeOH/DCM) which resulted in a tan amorphous solid (41 mg, 94%). TLC R_{f} 0.3 (5% MeOH/DCM); ¹H NMR (500 MHz, DMSO-d₆) δ 7.54 (t, J=8.0 Hz, 1H), 7.37 (s, 1H), 7.24 (d, J=8.0 Hz, 1H), 7.16 (d, J=8.0 Hz, 1H), 4.10-4.01 (m, 2H), 3.18 (t, J=8.0 Hz, 2H), 3.09 (s, 6H); ¹³C NMR (126 MHz, DMSO-d₆) δ 177.0, 163.0, 144.3, 141.9, 133.5, 132.7, 126.1, 115.1, 113.5, 93.6, 46.1, 22.5 (2C); HRMS (ESI) m/z: Calculated for C₁₄H₁₅³⁵ClN₃O [M+H]⁺ 276.0898, Observed: 276.0899. Analytical data are consistent with those reported.¹³

### (E)-6-Chloro-3-(2-ethylbutylidene)-2,3-dihydropyrrolo[1,2-a]quinazolin-5(1H)-one (7)

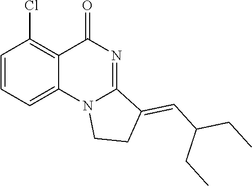 [SMILES: CCC(/C=C1\CCn2c1nc(=O)c1c(Cl)cccc12)CC]

The desired compound was obtained according to the reported procedure.¹³ To a stirred solution of 6-chloro-2,3-dihydropyrrolo[1,2-a]quinazolin-5(1H)-one (7c; 100 mg, 0.45 mmol) in CH2Cl2 (10 mL/mmol) was added t-BuOK (61 mg, 0.54 mmol). After 5 min, 2-ethylbutyraldehyde (61 μL, 0.54 mmol) was added. After completion of the reaction (approximately 1 h, as assessed by TLC), the crude mixture was diluted with CH₂Cl₂ and washed with NaHCO₃ (15 mL), and the aqueous layer extracted with CH2Cl2 (3′ 10 mL). The combined organic layer was dried (anhydrous MgSO₄) and concentrated under vacuum. The resulted residue was purified via MPLC (1% MeOH/DCM to 5% MeOH/DCM) and gave title compound as a white amorphous solid (80 mg, 60%). TLC R_{f} 0.3 (4% MeOH/DCM); ¹H NMR (500 MHz, DMSO-d₆) δ 7.69 (t, J=8.0 Hz, 1H), 7.46 (d, J=8.0 Hz, 1H), 7.43 (d, J=8.0 Hz, 1H), 6.52 (dt, J=10.5, 2.5 Hz, 1H), 4.25 (t, J=7.5, 2H), 2.95 (ddd, J=8.5, 6.5, 2.5 Hz, 2H), 2.21 (ddq, J=13.5, 9.0, 5.0 Hz, 1H), 1.53 (ddd, J=13.0, 7.5, 5.0 Hz, 2H), 1.35 (dt, J=13.5, 8.0 Hz, 2H), 0.85 (t, J=7.5 Hz, 6H); ¹³C NMR (126 MHz, DMSO-d₆) δ 166.8, 158.9, 141.3, 139.3, 133.7, 133.4, 132.8, 128.1, 115.7, 115.1, 46.4, 42.8, 26.9, 22.4 (2C), 11.7 (2C); HRMS (APCI) m/z: Calculated for C₁₇H₂₀³⁵ClN₂O [M+H]⁺ 303.1259, Observed: 303.1261; HPLC Rₜ26.97 min, purity 100%. Analytical data are consistent with those reported. ¹³

### (E)-3-(2-Ethylbutylidene)-2,3-dihydropyrrolo[1,2-a]quinazolin-5(1H)-one (8)

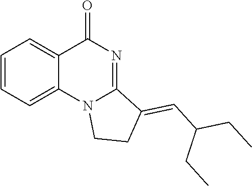 [SMILES: CCC(/C=C1\CCn2c1nc(=O)c1ccccc12)CC]

The desired compound was obtained according to the reported procedure.¹³ To a stirred solution of 2,3-dihydropyrrolo[1,2-a]quinazolin-5(1H)-one (Sc; 200 mg, 1.1 mmol) in CH2Cl2 (5 mL) was added t-BuOK (145 mg, 1.3 mmol). After 5 min, 2-ethylbutyraldehyde (0.15 mL, 1.2 mmol) was added. After completion of the reaction (approximately 1 h, as assessed by TLC), the crude mixture was diluted with CH₂Cl₂ and washed with NaHCO₃ (15 mL), and the aqueous layer extracted with CH2Cl2 (3′ 10 mL). The combined organic layer was dried (anhydrous MgSO₄) and concentrated under vacuum. The resulted residue was purified via MPLC (1% MeOH/DCM to 5% MeOH/DCM) and gave the title compound (100 mg, 34%). TLC R_{f} 0.70 (10% MeOH/DCM); ¹H NMR (500 MHz, DMSO-d₆) δ 8.04 (d, J=8.0 Hz, 1H), 7.78 (t, J=7.5 Hz, 1H), 7.56-7.41 (m, 2H), 6.53 (dd, J=10.5, 3.0 Hz, 1H), 4.26 (t, J=7.0 Hz, 2H), 2.96 (td, J=7.0, 2.5 Hz, 2H), 2.21 (tt, J=9.0, 4.5 Hz, 1H), 1.53 (dp, J=13.0, 7.0 Hz, 2H), 1.34 (dp, J=15.0, 7.5 Hz, 2H), 0.85 (t, J=7.5 Hz, 6H); ¹³C NMR (126 MHz, DMSO-d₆) δ 169.0, 159.6, 138.9, 138.8, 133.6, 133.2, 127.3, 125.7, 119.1, 115.8, 45.7, 42.8, 27.0, 22.6 (2C), 11.7 (2C); HRMS (APCI) m/z: Calculated for C₁₇H₂₁N₂O [M+H]⁺ 269.1648, Observed: 269.1651; HPLC Rₜ20.90 min, purity 100%. Analytical data are consistent with those reported.¹³

### 2-(2-Chloro-6-fluorophenyl)-2,3-dihydroquinazolin-4(1H)-one (5)

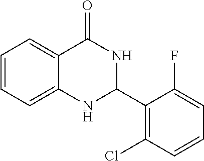 [SMILES: O=C1NC(c2c(F)cccc2Cl)Nc2ccccc21]

2-Aminobenzamide (9a; 136 μmg, 1 μmmol) and 2-chloro-6-fluorobenzaldehyde (187 mg, 1.2 mmol) were reacted according to general procedure B. Purification by MPLC (1% EtOAc/hexanes to 60% EtOAc/hexanes) gave a white solid (90 mg, 32%). TLC R_{f} 0.65 (50% EtOAc/hexanes); ¹H NMR (500 MHz, DMSO-d₆) δ 8.21 (s, 1H), 7.60 (dd, J=8.0, 1.5 Hz, 1H), 7.44 (td, J=8.0, 5.5 Hz, 1H), 7.34 (d, J=8.0 Hz, 1H), 7.27-7.18 (m, 2H), 7.05 (s, 1H), 6.64 (t, J=7.5 Hz, 1H), 6.60 (d, J=8.0 Hz, 1H), 6.43 (s, 1H); ¹³C NMR (126 MHz, DMSO-d₆) δ 162.8, 161.9 (d, ¹J_{CF}=253.0 Hz), 147.5, 133.4 (d, ³J_{CF}=6.0 Hz), 133.2, 131.1 (d, ³J_{CF}=10.0 Hz), 127.1, 125.9 (d, ⁴J_{CF}=3.0 Hz), 125.5 (d, ²J_{CF}=13.5 Hz), 116.7, 115.7 (d, ²J_{CF}=23.0 Hz), 113.8, 113.6, 62.0 (b.s); HRMS (APCI) m/z: Calculated for C₁₄H₁₁³⁵Cl¹⁹FN₂O [M+H]⁺ 277.0538, Observed: 277.0541; HPLC Rₜ 28.91 min, purity 95.9%. Analytical data are consistent with those reported.¹⁴

### 5-Chloro-2-(2-chloro-6-fluorophenyl)-2,3-dihydroquinazolin-4(1H)-one (11)

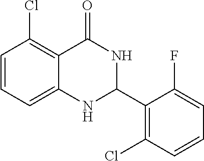 [SMILES: O=C1NC(c2c(F)cccc2Cl)Nc2cccc(Cl)c21]

2-Amino-6-chlorobenzamide (9b; 170 mg, 1 mmol) and 2-chloro-6-fluorobenzaldehyde (187 mg, 1.2 mmol) were reacted according to general procedure B. Purification by MPLC (1% EtOAc/hexanes to 40% EtOAc/hexanes) gave a white solid (100 mg, 32%). TLC R_{f} 0.70 (50% EtOAc/hexanes); ¹H NMR (500 MHz, DMSO-d₆) δ 8.34 (s, 1H), 7.45 (td, J=8.0, 5.5 Hz, 1H), 7.39-7.34 (m, 2H), 7.27 (dd, J=10.5, 8.0 Hz, 1H), 7.14 (t, J=8.0 Hz, 1H), 6.67 (d, J=8.0 Hz, 1H), 6.62 (d, J=8.0 Hz, 1H), 6.31 (s, 1H); ¹³C NMR (126 MHz, DMSO-d₆) δ 161.9 (d, ¹J_{CF}=253.0 Hz), 160.8, 150.4, 133.7, 133.5 (d, ³J_{CF}=6.0 Hz), 132.9, 131.3 (d, ³J_{CF}=10.0 Hz), 126.0 (d, ⁴J_{CF}=3.0 Hz), 124.5 (d, ²J_{CF}=14.0 Hz), 120.0, 115.7 (d, ²J_{CF}=23.0 Hz), 113.3, 111.0, 61.3 (b.s); HRMS (APCI) m/z: Calculated for C₁₄H₁₀³⁵Cl₂¹⁹FN₂O [M+H]⁺ 311.0149, Observed: 311.0152; HPLC Rₜ 28.46 min, purity 98.5%.

### 5-Chloro-2-phenyl-2,3-dihydroquinazolin-4(1H)-one (12)

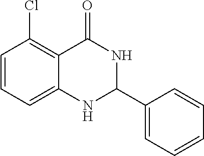 [SMILES: O=C1NC(c2ccccc2)Nc2cccc(Cl)c21]

2-Amino-6-chlorobenzamide (9b; 100 mg, 0.6 mmol) and benzaldehyde (0.07 mL, 0.7 mmol) were reacted according to general procedure B. Purification by MPLC (1% EtOAc/hexanes to 50% EtOAc/hexanes) gave a white solid (90 mg, 65%). TLC R_{f} 0.65 (50% EtOAc/hexanes); ¹H NMR (500 MHz, DMSO-d₆) δ 8.40 (bs, 1H), 7.49-7.41 (m, 3H), 7.38 (app t, J=7.0 Hz, 2H), 7.34 (d, J=7.0 Hz, 1H), 7.14 (t, J=8.0 Hz, 1H), 6.74 (d, J=8.0 Hz, 1H), 6.66 (d, J=7.5 Hz, 1H), 5.65 (s, 1H); ¹³C NMR (126 MHz, DMSO-d₆) δ 161.2, 150.6, 140.6, 133.7, 133.0, 128.5, 128.3, 126.9, 120.2, 113.9, 112.0, 65.5; HRMS (APCI) m/z: Calculated for C₁₄H₁₂³⁵ClN₂O [M+H]⁺ 259.0633, Observed: 259.0635; HPLC R_{f} 25.56 min, purity 98.7%.

### 5-Chloro-2-(2-fluorophenyl)-2,3-dihydroquinazolin-4(1H)-one (13)

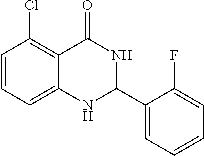 [SMILES: O=C1NC(c2ccccc2F)Nc2cccc(Cl)c21]

2-Amino-6-chlorobenzamide (9b; 100 μmg, 0.6 μmmol) and 2-fluorobenzaldehyde (0.07 mL, 0.7 mmol) were reacted according to general procedure B. Purification by MPLC (1% EtOAc/hexanes to 60% EtOAc/hexanes) gave a white solid (70 mg, 42%). TLC R_{f} 0.75 (50% EtOAc/hexanes); ¹H NMR (500 MHz, DMSO-d₆) δ 8.37 (s, 1H), 7.54-7.48 (m, 1H), 7.43-7.41 (m, 1H), 7.35 (s, 1H), 7.26-7.20 (m, 2H), 7.16 (t, J=8.0 Hz, 1H), 6.74 (d, J=8.0 Hz, 1H), 6.69 (d, J=7.5 Hz, 1H), 5.93 (s, 1H); ¹³C NMR (126 MHz, DMSO-d₆) δ 161.2, 159.8 (d, ¹J_{CF}=247.5 Hz), 150.4, 133.8, 133.0, 130.6 (d, ³J_{CF}=8.0 Hz), 128.4 (d, ³J_{CF}=3.5 Hz), 127.2 (d, ²J_{CF}=12.5 Hz), 124.4 (d, ⁴J_{CF}=3.5 Hz), 120.4, 115.6 (d, ²J_{CF}=22.0 Hz), 113.8, 111.8, 60.1 (d, ³J_{CF}=3.5 Hz); LRMS (APCI) m/z: Calculated for C₁₄H₁₁³⁵Cl¹⁹FN₂O [M+H]⁺ 277.1, Observed: 277.1; HPLC Rₜ 25.56 min, purity 98.4%.

### 5-Chloro-2-(2-chlorophenyl)-2,3-dihydroquinazolin-4(1H)-one (14)

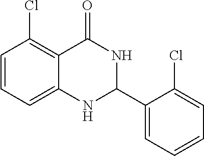 [SMILES: O=C1NC(c2ccccc2Cl)Nc2cccc(Cl)c21]

2-Amino-6-chlorobenzamide (9b; 100 μmg, 0.6 μmmol) and 2-chlorobenzaldehyde (0.08 mL, 0.7 mmol) were reacted according to general procedure B. Purification by MPLC (1% EtOAc/hexanes to 40% EtOAc/hexanes) gave a white solid (60 mg, 34%). TLC R_{f}0.7 (50% EtOAc/hexanes); ¹H NMR (500 MHz, DMSO-d₆) δ 8.33 (s, 1H), 7.66-7.61 (m, 1H), 7.51-7.46 (m, 1H), 7.42-7.38 (m, 2H), 7.31 (s, 1H), 7.17 (t, J=8.0 Hz, 1H), 6.75 (d, J=8.0 Hz, 1H), 6.72 (d, J=7.5 Hz, 1H), 6.00 (s, 1H); ¹³C NMR (126 MHz, DMSO-d₆) δ 161.2, 150.5, 136.8, 133.8, 133.1, 132.0, 130.4, 129.6, 128.7, 127.4, 120.5, 114.0, 111.8, 62.8; HRMS (APCI) m/z: Calculated for C₁₄H₁₁³⁵Cl¹⁹FN₂O [M+H]⁺ 293.0243, Observed: 293.0247; HPLC Rₜ 27.43 min.

### 5-Chloro-2-(2-methylphenyl)-2,3-dihydroquinazolin-4(1H)-one (15)

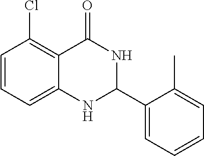 [SMILES: Cc1ccccc1C1NC(=O)c2c(Cl)cccc2N1]

2-Amino-6-chlorobenzamide (9b; 100 μmg, 0.6 μmmol) and 2-methylbenzaldehyde (0.07 mL, 0.7 mmol) were reacted according to general procedure B. Purification by MPLC (1% EtOAc/hexanes to 70% EtOAc/hexanes) gave a white solid (100 mg, 62%). TLC R_{f} 0.7 (50% EtOAc/hexanes); ¹H NMR (500 MHz, DMSO-d₆) δ 8.18 (s, 1H), 7.53 (dd, J=7.5, 2.0 Hz, 1H), 7.29-7.18 (m, 3H), 7.18-7.13 (m, 2H), 6.74 (d, J=8.0 Hz, 1H), 6.70 (d, J=8.0 Hz, 1H), 5.86 (s, 1H), 2.40 (s, 3H); ¹³C NMR (126 MHz, DMSO-d₆) δ 161.6, 151.4, 136.9, 136.2, 133.8, 132.8, 130.6, 128.5, 127.4, 125.8, 120.2, 113.9, 112.1, 63.7, 18.7; HRMS (APCI) m/z: Calculated for C₁₅H₁₄³⁵ClN₂O [M+H]⁺ 273.0789, Observed: 273.0792; HPLC Rₜ 27.64 min, purity 96.2%.

### 5-Chloro-2-(3-methylphenyl)-2,3-dihydroquinazolin-4(1H)-one (16)

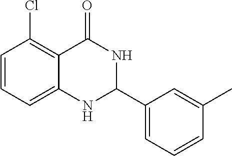 [SMILES: Cc1cccc(C2NC(=O)c3c(Cl)cccc3N2)c1]

2-Amino-6-chlorobenzamide (9b; 100 μmg, 0.6 μmmol) and 3-methylbenzaldehyde (0.1 mL, 0.7 mmol) were reacted according to general procedure B. Purification by MPLC (1% EtOAc/hexanes to 60% EtOAc/hexanes) gave a white solid (40 mg, 25%). TLC R_{f} 0.4 (50% EtOAc/hexanes); ¹H NMR (500 MHz, DMSO-d₆) δ 8.34 (s, 1H), 7.37 (s, 1H), 7.30-7.23 (m, 3H), 7.17-7.12 (m, 2H), 6.73 (d, J=8.0 Hz, 1H), 6.66 (d, J=8.0 Hz, 1H), 5.60 (s, 1H), 2.30 (s, 3H); ¹³C NMR (126 MHz, DMSO-d₆) δ 161.2, 150.6, 140.5, 137.4, 133.7, 132.9, 129.1, 128.2, 127.5, 124.0, 120.1, 113.9, 112.0, 65.6, 21.0; HRMS (APCI) m/z: Calculated for C₁₅H₁₄³⁵ClN₂O [M+H]⁺ 273.0789, Observed: 273.0790; HPLC R_{f} 26.89 min, purity 95.4%.

### 5-Chloro-2-(4-methylphenyl)-2,3-dihydroquinazolin-4(1H)-one (17)

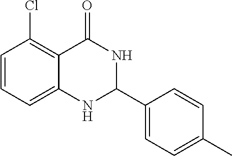 [SMILES: Cc1ccc(C2NC(=O)c3c(Cl)cccc3N2)cc1]

2-amino-6-chlorobenzamide (9b; 100 μmg, 0.6 μmmol) and 4-methylbenzaldehyde (0.07 mL, 0.7 mmol) were reacted according to general procedure B. Purification by MPLC (1% EtOAc/hexanes to 70% EtOAc/hexanes) gave a white solid (115 mg, 710%). TLC R_{f} 0.8 (50% EtOAc/hexanes); ¹H NMR (500 MHz, DMSO-d₆) δ 8.35 (s, 1H), 7.39-7.31 (m, 3H), 7.18 (d, J=8.0 Hz, 2H), 7.13 (t, J=8.0 Hz, 1H), 6.72 (d, J=8.0 Hz, 1H), 6.65 (d, J=8.0 Hz, 1H), 5.59 (s, 1H), 2.27 (s, 3H); ¹³C NMR (126 MHz, DMSO-d₆) δ 161.2, 150.6, 137.8, 137.6, 133.7, 132.9, 128.8, 126.8, 120.1, 113.9, 112.1, 65.3, 20.6; HRMS (APCI) m/z: Calculated for C₁₅H₁₄³⁵ClN₂O [M+H]⁺ 273.0789, Observed: 273.0792.

### 5-Chloro-2-(4-methoxyphenyl)-2,3-dihydroquinazolin-4(1H)-one (18)

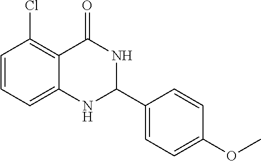 [SMILES: COc1ccc(C2NC(=O)c3c(Cl)cccc3N2)cc1]

2-Amino-6-chlorobenzamide (9b; 100 μmg, 0.6 μmmol) and 4-methoxybenzaldehyde (0.07 mL, 0.7 mmol) were reacted according to general procedure B. Purification by MPLC (1% EtOAc/hexanes to 60% EtOAc/hexanes) gave a white solid (108 mg, 62%). TLC R_{f} 0.8 (50% EtOAc/hexanes); ¹H NMR (500 MHz, DMSO-d₆) δ 8.30 (bs, 1H), 7.38 (d, J=8.5 Hz, 2H), 7.32 (s, 1H), 7.14 (t, J=8.0 Hz, 1H), 6.93 (d, J=8.5 Hz, 2H), 6.72 (d, J=8.0 Hz, 1H), 6.66 (d, J=8.0 Hz, 1H), 5.59 (s, 1H), 3.73 (s, 3H); ¹³C NMR (126 MHz, DMSO-d₆) δ 161.2, 159.4, 150.7, 133.7, 132.9, 132.4, 128.2, 120.1, 113.9, 113.6, 112.1, 65.2, 55.1; HRMS (APCI) m/z: Calculated for C₁₅H₁₄³⁵ClN₂O₂ [M+H]⁺ 289.0738, Observed: 289.074.

### 2-(2-Methylphenyl)-2,3-dihydroquinazolin-4(1H)-one (19)

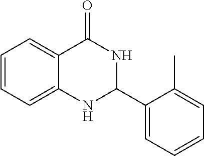 [SMILES: Cc1ccccc1C1NC(=O)c2ccccc2N1]

2-Aminobenzamide (9a; 100 mg, 0.6 mmol) and 2-methylbenzaldehyde (0.2 mL, 0.7 mmol) were reacted according to general procedure B. Purification by MPLC (1% EtOAc/hexanes to 50% EtOAc/hexanes) gave a white solid (40 mg, 28%). TLC R_{f}0.35 (50% EtOAc/hexanes); ¹H NMR (500 MHz, DMSO-d₆) δ 8.04 (bs, 1H), 7.64 (dd, J=7.5, 1.5 Hz, 1H), 7.55 (dd, J=7.5, 2.0 Hz, 1H), 7.30-7.15 (m, 4H), 6.85 (bs, 1H), 6.78-6.65 (m, 2H), 5.99 (s, 1H), 2.42 (s, 3H); ¹³C NMR (126 MHz, DMSO-d₆) δ 164.0, 148.5, 138.0, 136.0, 133.1, 130.6, 128.4, 127.4, 125.9, 117.1, 114.8, 114.4, 64.6, 18.7; HRMS (APCI) m/z: Calculated for C₁₅H₁₅N₂O [M+H]⁺ 239.1179, Observed: 239.1180; HPLC Rr 25.82 min, purity 97.2%.

### 5-Chloro-2-(3-(trifluoromethyl)phenyl)-2,3-dihydroquinazolin-4(1H)-one (20)

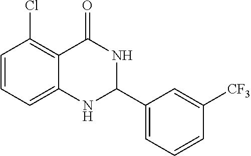 [SMILES: O=C1NC(c2cccc(C(F)(F)F)c2)Nc2cccc(Cl)c21]

2-Amino-6-chlorobenzamide (9b; 100 μmg, 0.6 μmmol) and 3-trifluoromethylbenzaldehyde (0.12 mL, 0.7 mmol) were reacted according to general procedure B. Purification by MPLC (1% EtOAc/hexanes to 50% EtOAc/hexanes) gave a white solid (60 mg, 30%). TLC R_{f} 0.4 (50% EtOAc/hexanes); ¹H NMR (500 MHz, DMSO-d₆) δ 8.53 (s, 1H), 7.82 (s, 1H), 7.77 (q, J=6.5 Hz, 2H), 7.75-7.69 (m, 1H), 7.63 (t, J=7.5 Hz, 1H), 7.53 (s, 1H), 6.75 (d, J=8.0 Hz, 1H), 6.69 (d, J=8.0 Hz, 1H), 5.79 (s, 1H); ¹³C NMR (126 MHz, DMSO-d₆) Major peaks are listed. δ 161.1, 150.4, 142.2, 133.8, 133.2, 131.1, 129.6, 125.3 (q, ³J_{CF}=4.0 Hz), 129.0 (q, ²J_{CF}=32.0 Hz), 124.1 (q, ¹J_{CF}=272.0 Hz), 123.7 (q, ³J_{CF}=4.0 Hz), 120.6, 114.1, 112.1, 64.8; HRMS (APCI) m/z: Calculated for C₁₅H₁₁³⁵Cl¹⁹F₃N₂O [M+H]⁺ 327.0507, Observed: 327.0508.

### 5-Chloro-2-(2,4-dimethylphenyl)-2,3-dihydroquinazolin-4(1H)-one (21)

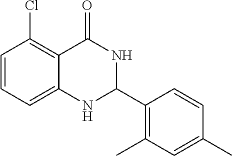 [SMILES: Cc1ccc(C2NC(=O)c3c(Cl)cccc3N2)c(C)c1]

2-Amino-6-chlorobenzamide (9b; 100 mg, 0.6 mmol) and 2,4-dimethylbenzaldehyde (0.1 mL, 0.7 mmol) were reacted according to general procedure B. Purification by MPLC (1% EtOAc/hexanes to 60% EtOAc/hexanes) gave a white solid (30 mg, 18%). TLC R_{f} 0.4 (50% EtOAc/hexanes); ¹H NMR (500 MHz, DMSO-d₆) δ 8.12 (s, 1H), 7.39 (d, J=8.0 Hz, 1H), 7.15 (t, J=8.0 Hz, 1H), 7.11 (s, 1H), 7.05-7.00 (m, 2H), 6.74 (d, J=8.0 Hz, 1H), 6.69 (d, J=8.0 Hz, 1H), 5.81 (s, 1H), 2.35 (s, 3H), 2.26 (s, 3H); ¹³C NMR (126 MHz, DMSO-d₆) δ 161.6, 151.4, 137.7, 136.0, 134.0, 133.8, 132.8, 131.2, 127.4, 126.3, 120.2, 113.9, 112.1, 63.6, 20.5, 18.6; HRMS (APCI) m/z: Calculated for C₁₆H₁₆³⁵ClN₂O [M+H]⁺ 287.0946, Observed: 287.0948; HPLC R_{f} 27.99 min.

### 5-Chloro-2-(2,4,6-trimethylphenyl)-2,3-dihydroquinazolin-4(1H)-one (22)

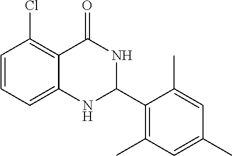 [SMILES: Cc1cc(C)c(C2NC(=O)c3c(Cl)cccc3N2)c(C)c1]

2-Amino-6-chlorobenzamide (9b; 100 mg, 0.6 mmol) and 2,4,6-trimethylbenzaldehyde (0.1 mL, 0.7 mmol) were reacted according to general procedure B. Purification by MPLC (1% EtOAc/hexanes to 50% EtOAc/hexanes) gave a white solid (20 mg, 11%). TLC R_{f} 0.55 (50% EtOAc/hexanes); ¹H NMR (500 MHz, DMSO-d₆) δ 8.03 (s, 1H), 7.13 (t, J=8.0 Hz, 1H), 7.04 (s, 1H), 6.86 (s, 2H), 6.68 (d, J=8.0 Hz, 2H), 6.09 (s, 1H), 2.41 (s, 6H), 2.22 (s, 3H); ¹³C NMR (126 MHz, DMSO-d₆) δ 161.8, 152.0, 137.8, 137.6, 133.9, 132.6, 130.3, 129.7, 120.0, 113.8, 111.9, 62.9, 20.44, 20.36; LRMS (APCI) m/z: Calculated for C₁₇H₁₈³⁵C₁N₂O [M+H]⁺ 301.1, Observed: 301.2; HPLC Rₜ 28.82 min, purity 95.7%.

### 5-Chloro-2-(3,5-dimethylphenyl)-2,3-dihydroquinazolin-4(1H)-one (23)

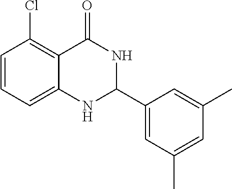 [SMILES: Cc1cc(C)cc(C2NC(=O)c3c(Cl)cccc3N2)c1]

2-Amino-6-chlorobenzamide (9b; 100 mg, 0.6 mmol) and 3,5-dimethylbenzaldehyde (0.1 mL, 0.7 mmol) were reacted according to general procedure B. Purification by MPLC (gradient 1% DCM/hexanes to 20% EtOAc/DCM) gave a white solid (30 mg, 18%). TLC R_{f} 0.45 (50% EtOAc/hexanes); ¹H NMR (500 MHz, CD₃COCD₃) δ 7.27-7.12 (m, 4H), 7.01 (s, 1H), 6.88-6.81 (m, 1H), 6.77 (d, J=8.0 Hz, 1H), 6.41 (s, 1H), 5.72 (s, 1H), 2.29 (s, 6H); ¹³C NMR (126 MHz, CD₃COCD₃) δ 160.8, 150.5, 139.2, 137.5, 134.4, 132.3, 129.9, 127.8, 124.5, 120.6, 113.4, 66.7, 19.8 (2C); HRMS (ESI⁻) m/z: Calculated for C₁₆H₁₄³⁵ClN₂O [M−H]⁻ 285.0789, Observed: 285.0793.

### 5-Chloro-2-(2,5-dimethylphenyl)-2,3-dihydroquinazolin-4(1H)-one (24)

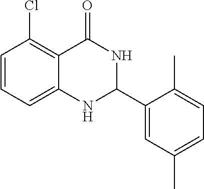 [SMILES: Cc1ccc(C)c(C2NC(=O)c3c(Cl)cccc3N2)c1]

2-Amino-6-chlorobenzamide (9b; 100 mg, 0.6 mmol) and 2,5-dimethylbenzaldehyde (0.1 mL, 0.7 mmol) were reacted according to general procedure B. Purification by MPLC (gradient 1% DCM/hexanes to 20% EtOAc/DCM) gave white solid (60 mg, 35%). TLC R_{f}0.35 (50% EtOAc/hexanes); ¹H NMR (500 MHz, DMSO-d₆) δ 8.11 (s, 1H), 7.37 (s, 1H), 7.15 (d, J=8.0 Hz, 1H), 7.12 (s, 1H), 7.09 (s, 2H), 6.74 (d, J=8.0 Hz, 1H), 6.70 (d, J=8.0 Hz, 1H), 5.82 (s, 1H), 2.33 (s, 3H), 2.27 (s, 3H); ¹³C NMR (126 MHz, DMSO-d₆) δ 162.2, 152.1, 137.0, 135.2, 134.4, 133.7, 133.4, 131.1, 129.7, 128.6, 120.8, 114.5, 112.7, 64.6, 21.2, 18.8; HRMS (APCI) m/z: C₁₆H₁₆³⁵ClN₂O [M+H]⁺ 287.0946, Observed: 287.0948.

### 5-Chloro-2-(2,3-dimethylphenyl)-2,3-dihydroquinazolin-4(1H)-one (25)

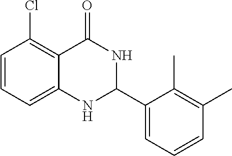 [SMILES: Cc1cccc(C2NC(=O)c3c(Cl)cccc3N2)c1C]

2-Amino-6-chlorobenzamide (9b; 100 mg, 0.6 mmol) and 2,3-dimethylbenzaldehyde (0.1 mL, 0.7 mmol) were reacted according to general procedure B. Purification by MPLC (1% EtOAc/hexanes to 60% EtOAc/hexanes) gave a white solid (42 mg, 24%). TLC R_{f} 0.45 (50% EtOAc/hexanes); ¹H NMR (500 MHz, DMSO-d₆) δ 8.14 (s, 1H), 7.37 (d, J=7.5 Hz, 1H), 7.22-7.08 (m, 4H), 6.74 (d, J=8.0 Hz, 1H), 6.70 (d, J=8.0 Hz, 1H), 5.90 (s, 1H), 2.28 (s, 3H), 2.25 (s, 3H); ¹³C NMR (126 MHz, DMSO-d₆) δ 162.1, 151.9, 137.4, 137.2, 135.4, 134.4, 133.4, 130.6, 125.9, 125.8, 120.8, 114.5, 112.7, 64.7, 20.7, 15.1; HRMS (APCI) m/z: C₁₆H₁₆³⁵ClN₂O [M+H]⁺ 287.0946, Observed: 287.0948.

### 5-Chloro-2-(2,6-dimethylphenyl)-2,3-dihydroquinazolin-4(1H)-one (26)

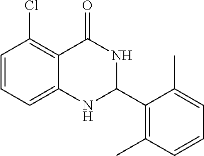 [SMILES: Cc1cccc(C)c1C1NC(=O)c2c(Cl)cccc2N1]

2-Amino-6-chlorobenzamide (9b; 100 mg, 0.6 mmol) and 2,6-dimethylbenzaldehyde (100 mg, 0.7 mmol) were reacted according to general procedure B. Purification by MPLC (gradient 1% DCM/hexanes to 20% EtOAc/DCM) gave a white solid (20 mg, 12%). TLC R_{f} 0.4 (50% EtOAc/hexanes); ¹H NMR (500 MHz, CD₃COCD₃) δ 7.20 (t, J=8.0 Hz, 1H), 7.14 (d, J=7.5 Hz, 1H), 7.07-6.98 (m, 3H), 6.82 (d, J=8.0 Hz, 1H), 6.78 (d, J=8.0 Hz, 1H), 6.38 (s, 1H), 6.22 (s, 1H), 2.53 (s, 6H); ¹³C NMR (126 MHz, CD₃COCD₃) δ 151.6, 137.8, 134.6, 132.6, 132.2, 128.9, 128.7, 128.3, 120.5, 113.4, 113.3, 63.4, 19.6 (2C); HRMS (APCI) m/z Calculated for C₁₆H₁₆³⁵ClN₂O [M+H]⁺ 287.0946, Observed: 287.0948; HPLC R27.02 min, purity 95.8%.

### (E)-2-((2-Chloro-6-fluorobenzylidene)amino)benzamide (27)

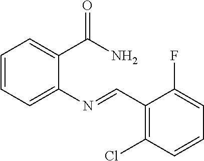 [SMILES: NC(=O)c1ccccc1/N=C/c1c(F)cccc1Cl]

o-Aminobenzamide (9a; 200 μmg, 1.5 μmmol) and 2-chloro-6-fluorobenzaldehyde (232 mg, 1.5 mmol) were added to toluene (20 mL) in a 20 mL microwave vial charged with a stir bar. The reaction was irradiated under microwave at 150° C. for 1 h. After the consumption of the starting material (as assessed by TLC), the reaction was cooled to room temperature and the crude product mixture was concentrated under reduced pressure, then diluted with ethyl acetate and washed with water (3×10 mL). The combined organic layer was dried (anhydrous MgSO₄) and concentrated under vacuum. The resulted residue was purified via MPLC (1% EtOAc/hexanes to 40% EtOAc/hexanes) which gave the title compound (100 mg, 25%). TLC R_{f} 0.55 (50% EtOAc/hexanes); ¹H NMR (500 MHz, DMSO-d₆) δ 8.78 (s, 1H), 8.36 (s, 1H), 7.99 (dd, J=8.0, 1.5 Hz, 1H), 7.70 (dt, J=4.0, 2.0 Hz, 1H), 7.65-7.56 (m, 2H), 7.51 (d, J=8.0 Hz, 1H), 7.45-7.38 (m, 2H), 7.25 (d, J=8.0 Hz, 1H); ¹³C NMR (126 MHz, DMSO-d₆) δ 166.5, 161.1 (d, ¹J_{CF}=257.0 Hz), 155.6 (d, ³J_{CF}=2.0 Hz), 149.0, 135.2 (d, ³J_{CF}=4.5 Hz), 133.7 (d, ²J_{CF}=10.5 Hz), 132.3, 130.1, 127.6, 126.9, 126.6 (d, ⁴J_{CF}=3.0 Hz), 121.1 (d, ³J_{CF}=11.5 Hz), 118.9, 116.0 (d, ²J_{CF}=22.0 Hz); HRMS (APCI) m/z: Calculated for C₁₄H₁₁³⁵Cl¹⁹FN₂O [M+H]⁺ 277.0538, Observed: 277.0541.

### (E)-2-Chloro-6-((2-chloro-6-fluorobenzylidene)amino)benzamide (28)

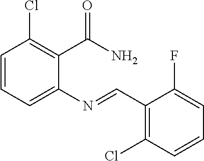 [SMILES: NC(=O)c1c(Cl)cccc1/N=C/c1c(F)cccc1Cl]

2-Amino-6-chlorobenzamide (9b; 93 mg, 0.5 mmol) and 2-chloro-6-fluorobenzaldehyde (86 mg, 0.5 mmol) were added to toluene (20 mL) in a 20 mL microwave vial charged with a stir bar. The reaction was irradiated under microwave at 150° C. for 1 h. After the consumption of the starting material (as assessed by TLC), the reaction was cooled to room temperature and the crude product mixture was concentrated under reduced pressure, then diluted with ethyl acetate and washed with water (3×10 mL). The combined organic layer was dried (anhydrous MgSO₄) and concentrated under vacuum. The purification via MPLC (1% EtOAc/hexanes to 40% EtOAc/hexanes) resulted in a white solid (100 mg, 60%). TLC R_{f} 0.40 (50% EtOAc/hexanes); ¹H NMR (500 MHz, DMSO-d₆) δ 8.65 (s, 1H), 7.80 (s, 1H), 7.57 (q, 1H), 7.50 (s, 1H), 7.48-7.32 (m, 4H), 7.11 (d, J=8.0 Hz, 1H); ¹³C NMR (126 MHz, DMSO-d₆) δ 166.7, 161.4 (d, ¹J_{CF}=259.0 Hz), 156.6 (d, ³J_{CF}=2.0 Hz), 150.5, 135.3 (d, ³J_{CF}=4.0 Hz), 133.8 (d, ²J_{CF}=10.0 Hz), 132.4, 130.7, 130.4, 126.9, 126.7 (d, ⁴J_{CF}=3.5 Hz), 122.1 (d, ³J_{CF}=12.0 Hz), 117.7, 116.2 (d, ²J_{CF}=22.0 Hz); HRMS (ESI) m/z: Calculated for C₁₄H₉³⁵Cl₂¹⁹FN₂ONa [M+Na]⁺332.9968, Observed: 332.9970.

### 5-Chloro-2-(3-methylphenyl)-4H-benzo[d][1,3]oxazin-4-one (29)

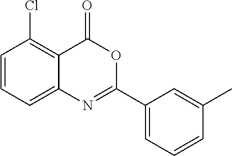 [SMILES: Cc1cccc(-c2nc3cccc(Cl)c3c(=O)o2)c1]

The compound was prepared according to a modified procedure.¹⁰ 3-methylbenzoyl chloride (0.8 mL, 2 mmol) in CH₂Cl₂ (2 mL) was added into a solution of 2-amino 5-chlorobenzoic acid (0.17 g, 1 mmol) and diisopropylethylamine (0.3 mL) in dichloromethane (2 mL) at 0° C. The reaction mixture was stirred overnight at room temperature and then condensed in vacuo. The residue was dissolved in anhydrous DMF (2 mL) followed by addition of diisopropylethylamine (0.3 mL) and HATU (0.4 g, 1 mmol). After stirring for 1 h at room temperature, the reaction was quenched with cold water (100 mL) at 0° C. The precipitate was collected and purified by MPLC (1% EtOAc/hexanes to 20% EtOAc/hexanes) to give the title compound (0.1 g, 37%). TLC R_{f}0.65 (50% EtOAc/hexanes); ¹H NMR (500 MHz, DMSO-d₆) δ 7.99-7.94 (m, 2H), 7.84 (t, J=8.0 Hz, 1H), 7.64 (dd, J=8.0, 2.0 Hz, 2H), 7.47 (d, J=5.0 Hz, 2H), 2.41 (s, 3H); ¹³C NMR (126 MHz, DMSO-d₆) δ 157.6, 156.2, 149.4, 139.0, 137.1, 134.6, 134.2, 131.0, 130.1, 129.5, 128.7, 126.8, 125.7, 115.3, 21.4; HRMS (APCI) m/z: Calculated for C₁₅H₁₁³⁵ClN₂O [M+H]⁺ 272.0473, Observed: 272.0475; HPLC Rₜ28.51 min.

### 5-Chloro-2-(3-methylphenyl)quinazolin-4(3H)-one (30)

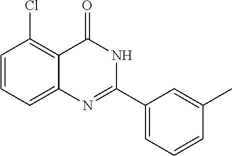 [SMILES: Cc1cccc(-c2nc3cccc(Cl)c3c(=O)[nH]2)c1]

5-Chloro-2-(3-methylphenyl)-4H-benzo[d][1,3]oxazin-4-one (29; 100 mg, 0.37 mmol) was suspended in 3 mL of an ammonium hydroxide solution (30-33% NH₃ in H₂O) and heated to 100° C. for 4 h. The crude mixture was concentrated under reduced pressure and the resulting precipitate was filtered and dried. The crude products were subsequently recrystallized from methanol and gave the title compound as a white solid (60 mg, 60%). TLC R_{f} 0.6 (50% EtOAc/hexanes); ¹H NMR (500 MHz, DMSO-d₆) δ 12.46 (s, 1H), 8.00 (s, 1H), 7.94 (d, J=7.0 Hz, 1H), 7.72 (t, J=8.0 Hz, 1H), 7.66 (d, J=8.0 Hz, 1H), 7.49 (d, J=7.5 Hz, 1H), 7.41 (d, J=7.5 Hz, 2H), 2.39 (s, 3H); ¹³C NMR (126 MHz, DMSO-d₆) δ 160.8, 153.6, 151.8, 138.4, 134.9, 133.0, 132.8, 132.5, 129.3, 129.0, 128.8, 127.6, 125.5, 118.4, 21.4; HRMS (APCI) m/z: Calculated for C₁₅H₁₂³⁵ClN₂O [M+H]⁺ 271.0633, Observed: 271.0635; HPLC Rₜ 29.05 min.

### 5-Methoxy-2-(2-methylphenyl)-2,3-dihydroquinazolin-4(1H)-one (31)

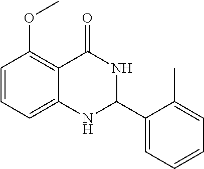 [SMILES: COc1cccc2c1C(=O)NC(c1ccccc1C)N2]

2-Amino-6-methoxybenzamide (9c; 332 μmg, 2 μmmol) and 2-methylbenzaldehyde (0.1 mL, 0.7 mmol) were reacted according to general procedure B. Purification by MPLC (gradient 1% DCM/hexanes to 20% EtOAc/DCM) gave a white solid (200 mg, 37%). ¹H NMR (500 MHz, DMSO-d₆) δ 7.74 (s, 1H), 7.52 (dd, J=7.5, 2.0 Hz, 1H), 7.27-7.16 (m, 3H), 7.13 (t, J=8.0 Hz, 1H), 6.83 (s, 1H), 6.37 (d, J=8.0 Hz, 1H), 6.29 (d, J=8.0 Hz, 1H), 5.75 (s, 1H), 3.70 (s, 3H), 2.38 (s, 3H); ¹³C NMR (126 MHz, DMSO) δ 162.4, 160.6, 151.0, 137.5, 136.1, 133.2, 130.5, 128.3, 127.3, 125.8, 107.3, 104.5, 101.2, 63.8, 55.3, 18.7; HRMS (ESI) m/z: Calculated for C₁₆H₁₆N₂O₂Na [M+Na]⁺291.1104, Observed: 291.1106.

### 5-Fluoro-2-(3-methylphenyl)-2,3-dihydroquinazolin-4(1H)-one (32)

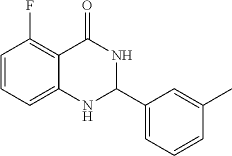 [SMILES: Cc1cccc(C2NC(=O)c3c(F)cccc3N2)c1]

2-Amino-6-fluorobenzamide (200 μmg, 1.3 μmmol) and 3-methylbenzaldehyde (0.2 mL, 1.6 mmol) were reacted according to general procedure B. Purification by MPLC (1% EtOAc/hexanes to 60% EtOAc/hexanes) gave a white solid (65 mg, 20%). TLC R_{f} 0.5 (50% EtOAc/hexanes); ¹H NMR (500 MHz, DMSO-d₆) δ 8.24 (s, 1H), 7.39 (s, 1H), 7.30-7.23 (m, 3H), 7.18 (ddd, J=19.5, 10.0, 6.0 Hz, 2H), 6.56 (d, J=8.0 Hz, 1H), 6.37 (dd, J=11.5, 8.0 Hz, 1H), 5.64 (s, 1H), 2.30 (s, 3H); ¹³C NMR (126 MHz, DMSO-d₆) δ 162.2 (d, ¹J_{CF}=259.0 Hz), 160.8 (d, ³J_{CF}=2.5 Hz), 150.2 (d, ³J_{CF}=2.5 Hz), 140.8, 137.4, 133.9 (d, ²J_{CF}=11.5 Hz), 129.2, 128.2, 127.5, 124.0, 110.4 (d, ⁴J_{CF}=3.5 Hz), 104.5 (d, ²J_{CF}=21.5 Hz), 103.6 (d, ³J_{CF}=9.0 Hz), 66.0, 21.0; HRMS (APCI) m/z: Calculated for C₁₅H₁₄¹⁹FN₂O [M+H]⁺ 257.1085, Observed: 257.1086.

### 5-Methyl-2-(3-methylphenyl)-2,3-dihydroquinazolin-4(1H)-one (33)

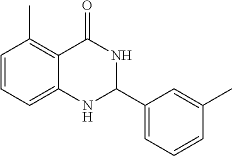 [SMILES: Cc1cccc(C2NC(=O)c3c(C)cccc3N2)c1]

2-Amino-6-methylbenzamide (9d; 100 μmg, 0.7 μmmol) and 3-methylbenzaldehyde (0.1 mL, 0.8 mmol) were reacted according to general procedure B. Purification by MPLC (gradient 1% DCM/hexanes to 20% EtOAc/DCM) gave a white solid (80 mg, 53%). TLC R_{f} 0.5 (50% EtOAc/hexanes); ¹H NMR (500 MHz, DMSO-d₆) δ 8.07 (s, 1H), 7.30 (s, 1H), 7.28-7.22 (m, 2H), 7.17-7.11 (m, 1H), 7.05 (t, J=8.0 Hz, 1H), 6.92 (s, 2H), 6.61 (d, J=8.0 Hz, 1H), 6.44 (d, J=7.45 Hz, 1H), 2.50 (s, 3H), 2.30 (s, 3H); ¹³C NMR (126 MHz, DMSO-d₆) δ 164.9, 149.8, 141.7, 140.8, 137.8, 132.6, 129.5, 128.7, 128.1, 124.6, 121.4, 114.0, 113.4, 66.6, 22.5, 21.6; HRMS (APCI) m/z: Calculated for C₁₆H₁₇N₂O [M+H]⁺ 253.1335, Observed: 253.1336.

### 5-Bromo-2-(3-methylphenyl)-2,3-dihydroquinazolin-4(1H)-one (34)

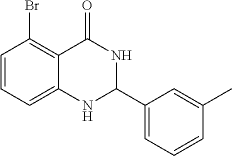 [SMILES: Cc1cccc(C2NC(=O)c3c(Br)cccc3N2)c1]

2-Amino-6-bromobenzamide (9e; 100 μmg, 0.5 μmmol) and 3-methylbenzaldehyde (0.1 mL, 0.6 mmol) were reacted according to general procedure B. Purification by MPLC (1% EtOAc/hexanes to 50% EtOAc/hexanes) gave a white solid (40 mg, 25%). TLC R_{f} 0.6 (50% EtOAc/hexanes); ¹H NMR (500 MHz, DMSO-d₆) δ 8.37 (s, 1H), 7.37 (s, 1H), 7.33-7.22 (m, 3H), 7.16 (d, J=6.5 Hz, 1H), 7.05 (t, J=8.0 Hz, 1H), 6.87 (d, J=8.0 Hz, 1H), 6.78 (d, J=8.0 Hz, 1H), 5.60 (s, 1H), 2.30 (s, 3H); ¹³C NMR (126 MHz, DMSO) δ 161.8, 151.2, 141.0, 138.0, 133.8, 129.7, 128.8, 128.1, 124.6, 124.3, 122.4, 115.1, 113.6, 66.0, 21.5; HRMS (APCI) m/z: Calculated for C₁₅H₁₄⁷⁹BrN₂O [M+H]⁺ 317.0284, Observed: 317.0287; HPLC Rₜ 27.52 min, purity 95.92%.

### 2-((2-Carboxyphenyl)amino)-6-chlorobenzoic acid (35)

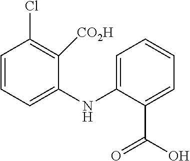 [SMILES: O=C(O)c1ccccc1Nc1cccc(Cl)c1C(=O)O]

The desired compound was obtained according to the reported procedure. ¹² To a stirred suspension of 2-amino-6-chlorobenzoic acid (860 mg, 5 mmol), 2-bromobenzoic acid (1.1 mg, 5.5 mmol), and K₂CO₃ (1.4 g, 10 mmol) was added copper powder (63 mg, 1 mmol) in EtOH (10 mL/mmol). The suspension was heated to reflux for 1.5 h. After completion of the reaction (as assessed by TLC), the mixture was filtered on celite to remove the copper. The filter bed was washed with H₂O and the resulting solution was acidified with concentrated HCl to a pH of 2-3. The resulting suspension was stirred for 1 h at 10° C., the solid was filtered and washed with H₂O. The product was recrystallized in EtOH/H₂O and dried under vacuum. The title product was obtained as a white solid (1.2 g, 83%). ¹H NMR (500 MHz, DMSO-d₆) δ 13.80 (bs, 1H), 13.20 (bs, 1H), 9.89 (s, 1H), 7.89 (d, J=7.5 Hz, 1H), 7.45-7.34 (m, 3H), 7.19 (d, J=8.0 Hz, 1H), 7.16 (d, J=8.0 Hz, 1H), 6.84 (t, J=7.5 Hz, 1H); ¹³C NMR (126 MHz, DMSO-d₆) δ 169.5, 166.6, 145.8, 139.2, 134.0, 131.7, 130.8, 130.1, 128.3, 123.9, 120.0, 118.6, 114.7, 113.7; HRMS (ESI⁻) m/z: Calculated for C₁₄H₉³⁵ClNO₄ [M−H]⁻ 290.0226, Observed: 290.0220.

### 8-Chloro-9-oxo-9,10-dihydroacridine-4-carboxylic acid (36)

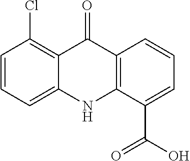 [SMILES: O=C(O)c1cccc2c(=O)c3c(Cl)cccc3[nH]c12]

The desired compound was obtained according to the reported procedure.¹² To a stirred suspension of 2-((2-carboxyphenyl)amino)-6-chlorobenzoic acid (36; 1.45 g, 5 mmol) in CH₃CN at reflux was added phosphorus(V)oxychloride (1 mL, 11 mmol) over a period of 1 h. The solution was refluxed till the consumption of starting material (approximately 2 h, as assessed by TLC) and then cooled to 10-15° C. H₂O (10 mL) was added and the mixture was heated to reflux for an additional 2.5 h. The suspension was cooled to 10° C. and filtered. The solid was washed with H₂O and CH₃CN and then dried under vacuum. The title product was obtained as white solid (1.0 g, 73%). ¹H NMR (500 MHz, DMSO-d₆) δ 13.82 (bs, 1H), 11.87 (s, 1H), 8.47-8.35 (m, 2H), 7.72-7.58 (m, 2H), 7.38-7.24 (m, 2H); ¹³C NMR (126 MHz, DMSO-d₆) δ 175.4, 168.9, 142.3, 140.3, 136.7, 133.5, 132.9, 132.5, 124.8, 122.7, 120.6, 118.1, 116.7, 114.7; HRMS (ESI⁻) m/z: Calculated for C₁₄H₇³⁵ClNO₃ [M−H]⁻ 272.0127, Observed: 272.0114.

### 2-((2-Carboxyphenyl)amino)-4-chlorobenzoic acid (37)

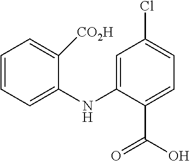 [SMILES: O=C(O)c1ccccc1Nc1cc(Cl)ccc1C(=O)O]

The desired compound was obtained according to the reported procedure.¹² To a stirred suspension of 2-aminobenzoic acid (1.3 g, 10 mmol), 2-bromo-4-chlorobenzoic acid (2.5 g, 11 mmol), and K₂CO₃ (2.77 g, 20 mmol) was added copper powder (0.125 g, 2 mmol) in EtOH (20 mL) The suspension was heated to reflux for 1.5 h. After completion of the reaction (as assessed by TLC), the mixture was filtered on celite to remove the copper. The filter bed was washed with H₂O and the resulting solution was acidified with concentrated HCl to a pH of 2-3. The resulting suspension was stirred for 1 h at 10° C., the solid was filtered and washed with H₂O. The product was recrystallized in EtOH/H₂O and dried under vacuum. The title product was obtained as a white solid (1.6 g, 28%). ¹H NMR (500 MHz, DMSO-d₆) δ 7.89 (app t, J=8.0 Hz, 2H), 7.54-7.46 (m, 2H), 7.34 (d, J=2.0 Hz, 1H), 7.06-6.99 (m, 1H), 6.91 (dd, J=8.5, 2.0 Hz, 1H), —COOH protons were not observed; ¹³C NMR (126 MHz, DMSO-d₆) δ 168.1, 167.8, 145.4, 142.1, 137.8, 133.6, 133.2, 131.7, 121.2, 119.5, 119.0, 118.8, 115.7, 115.3; HRMS (ESI⁻) m/z: Calculated for C₁₄H₉³⁵ClNO₄ [M−H]⁻ 290.0226, Observed: 290.0219.

### 1-Chloro-9-oxo-9,10-dihydroacridine-4-carboxylic acid (38)

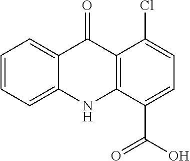 [SMILES: O=C(O)c1ccc(Cl)c2c(=O)c3ccccc3[nH]c12]

The desired compound was obtained according to the reported procedure.¹² To a stirred suspension of 2-((2-carboxyphenyl)amino)-4-chlorobenzoic acid (38; 400 mg, 1.4 mmol) in CH₃CN at reflux was added phosphorus(V)oxychloride (0.3 mL, 3 mmol) over a period of 1 h. The solution was refluxed till the consumption of starting material (approximately 2 h, as assessed by TLC) and then cooled to 10-15° C. H₂O (10 mL) was added and the mixture was heated to reflux for an additional 2.5 h. The suspension was cooled to 10° C. and filtered. The solid was washed with H₂O and CH₃CN and then dried under vacuum. ¹H NMR (500 MHz, DMSO-d₆) δ 13.82 (bs, 1H), 11.87 (s, 1H), 8.47-8.35 (m, 2H), 7.72-7.58 (m, 2H), 7.38-7.24 (m, 2H); ¹³C NMR (126 MHz, DMSO-d₆) δ 175.4, 168.9, 142.3, 140.3, 136.7, 133.5, 132.9, 132.5, 124.8, 122.7, 120.6, 118.1, 116.7, 114.7; HRMS (ESI⁻) m/z: Calculated for C₁₄H₇³⁵ClNO₃ [M−H]⁻ 272.0120, Observed: 272.0115.

### REFERENCES

1. Bradford, M. M. A rapid and sensitive method for the quantitation of microgram quantities of protein utilizing the principle of protein-dye binding. Analytical Biochemistry 1976, 72, 248-254.
2. Egner, J. M.; Jensen, D. R.; Olp, M. D.; Kennedy, N. W.; Volkman, B. F.; Peterson, F. C.; Smith, B. C.; Hill, R. B. Development and Validation of 2D Difference Intensity Analysis for Chemical Library Screening by Protein-Detected NMR Spectroscopy. Chembiochem 2018, 19, 448-458.
3. Charlop-Powers, Z.; Zeng, L.; Zhang, Q.; Zhou, M. M. Structural insights into selective histone H₃ recognition by the human Polybromo bromodomain 2. Cell Res 2010, 20, 529-38.
4. Delaglio, F.; Grzesiek, S.; Vuister, G.; Zhu, G.; Pfeifer, J.; Bax, A. NMRPipe: A multidimensional spectral processing system based on UNIX pipes. Journal of Biomolecular NMR 1995, 6.
5. Maciejewski, M. W.; Schuyler, A. D.; Gryk, M. R.; Moraru, I. I.; Romero, P. R.; Ulrich, E. L.; Eghbalnia, H. R.; Livny, M.; Delaglio, F.; Hoch, J. C. NMRbox: A Resource for Biomolecular NMR Computation. Biophysical Journal 2017, 112, 1529-1534.
6. Lee, W.; Tonelli, M.; Markley, J. L. NMRFAM-SPARKY: enhanced software for biomolecular NMR spectroscopy. Bioinformatics 2015, 31, 1325-1327.
7. Roos K, W. C., Damm W, Reboul M, Stevenson J M, Lu C, Dahlgren M K, Mondal S, Chen W, Wang L, Abel R, Friesner R A, Harder E D. OPLS3e: Extending Force Field Coverage for Drug-Like Small Molecules. Journal of Chemical Theory and Computation 2019, 15, 1863.
8. Greenwood, J. R.; Calkins, D.; Sullivan, A. P.; Shelley, J. C. Towards the comprehensive, rapid, and accurate prediction of the favorable tautomeric states of drug-like molecules in aqueous solution. Journal of Computer-Aided Molecular Design 2010, 24, 591-604.
9. Friesner, R. A.; Murphy, R. B.; Repasky, M. P.; Frye, L. L.; Greenwood, J. R.; Halgren, T. A.; Sanschagrin, P. C.; Mainz, D. T. Extra Precision Glide: Docking and Scoring Incorporating a Model of Hydrophobic Enclosure for Protein-Ligand Complexes. Journal of Medicinal Chemistry 2006, 49, 6177-6196.
10. Jin, S.; Liu, Z.; Milbum, C.; Tomaszewski, M.; Walpole, C.; Wei, Z.-Y.; Yang, H. Preparation of benzamide derivatives for therapeutic use as cannabinoid receptor modulators. WO2005115972A1, 2005.
11. Pieterse, L.; Van Der Walt, M. M.; Terre'Blanche, G. C2-substituted quinazolinone derivatives exhibit A1 and/or A2A adenosine receptor affinities in the low micromolar range. Bioorganic & Medicinal Chemistry Letters 2020, 30, 127274.
12. Dörner, B.; Kuntner, C.; Bankstahl, J. P.; Wanek, T.; Bankstahl, M.; Stanek, J.; Müllauer, J.; Bauer, F.; Mairinger, S.; Löscher, W.; Miller, D. W.; Chiba, P.; Müller, M.; Erker, T.; Langer, O. Radiosynthesis and in vivo evaluation of 1-[18F]fluoroelacridar as a positron emission tomography tracer for P-glycoprotein and breast cancer resistance protein. Bioorganic & Medicinal Chemistry 2011, 19, 2190-2198.
13. Sutherell, C. L.; Tallant, C.; Monteiro, O. P.; Yapp, C.; Fuchs, J. E.; Fedorov, O.; Siejka, P.; Mtiller, S.; Knapp, S.; Brenton, J. D.; Brennan, P. E.; Ley, S. V. Identification and Development of 2,3-Dihydropyrrolo[1,2-a]quinazolin-5(1H)-one Inhibitors Targeting Bromodomains within the Switch/Sucrose Nonfermenting Complex. Journal of Medicinal Chemistry 2016, 59, 5095-5101.
14. Dutta, A.; Damarla, K.; Bordoloi, A.; Kumar, A.; Sarma, D. KOH/DMSO: A basic suspension for transition metal-free Tandem synthesis of 2,3-dihydroquinazolin-4(1H)-ones. Tetrahedron Letters 2019, 60, 1614-1619.
15. Sutherell, C.; Ley, S. On the Synthesis and Reactivity of 2,3-Dihydropyrrolo[1,2-a]quinazolin-5(1H)-ones. Synthesis 2016, 49, 135-144.

1. A compound of formula (I): 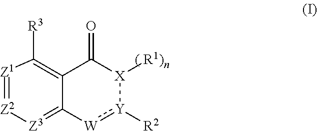
wherein X is selected from C, N, NH, and 0;
Y is selected from C and phenylene optionally substituted with halogen;
W is N or NH;
Z¹, Z², and Z³ are independently selected from CH and N;
n is 0 or 1;
R¹ is hydrogen, and R² is selected from the group consisting of carboxyl, pyridyl optionally substituted with one or more alkyl, and phenyl optionally substituted with one or more substituents selected from the group consisting of halogen, alkyl, alkoxy, and haloalkyl, or R¹ and R² together form a phenyl optionally substituted with one or more substituents selected from the group consisting of carboxyl and halogen; and
R³ is selected from the group consisting of hydrogen, alkoxy, alkyl, and halogen.

2. The compound of claim 1, having a formula I(a): 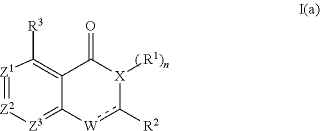
wherein X is N or O; and
R² is selected from pyridyl optionally substituted with one or more alkyl, and phenyl optionally substituted with one or more substituents selected from the group consisting of halogen, alkyl, alkoxy, and haloalkyl.

3. The compound of claim 2, having a formula I(b) 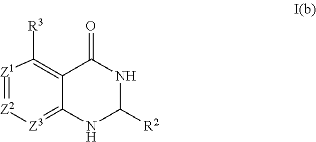
wherein R² is selected from the group consisting of pyridyl optionally substituted with one or more alkyl and phenyl optionally substituted with one or more substituents selected from the group consisting of halogen, alkyl, alkoxy, and haloalkyl.

4. The compound of claim 3, wherein R² is phenyl optionally substituted with one or more substituents selected from the group consisting of fluoro, chloro, methyl, methoxy, and trifluoromethyl;
R³ is hydrogen, fluoro, chloro, bromo, or methyl; and
Z¹, Z², and Z³ are CH.

5. The compound of claim 4, wherein the compound is

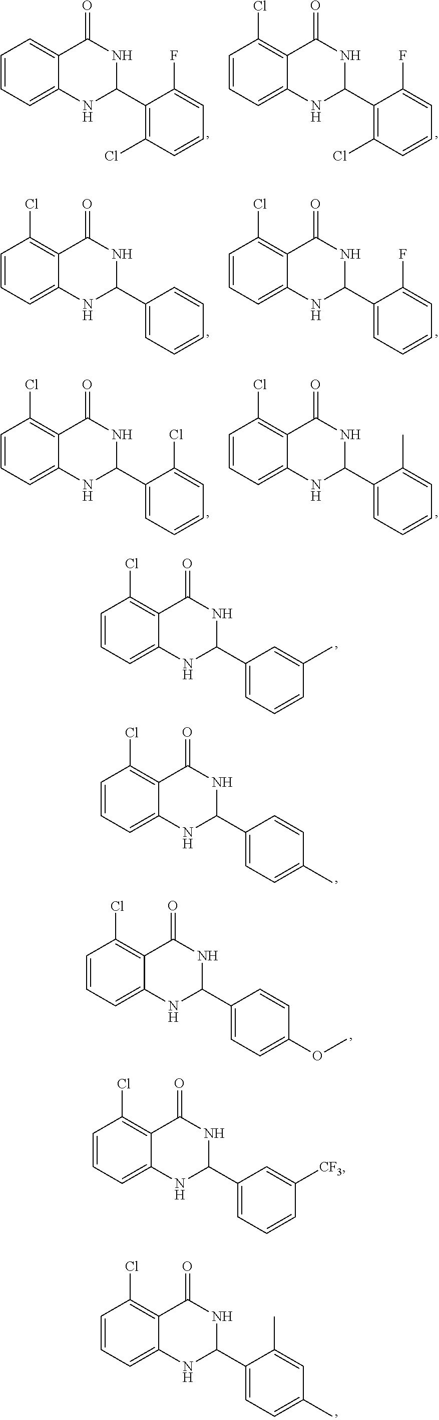

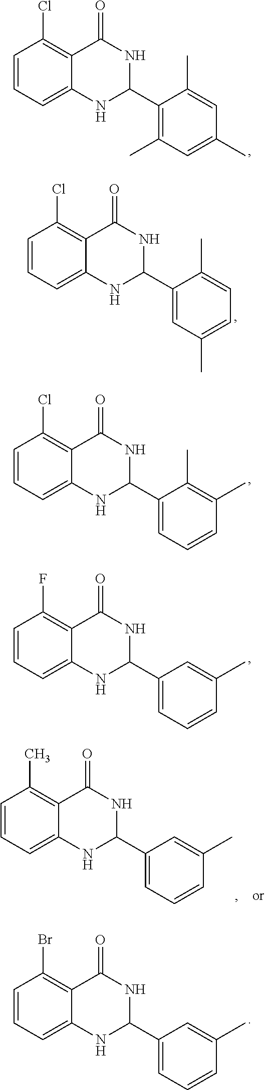

6. The compound of claim 3, wherein R² is pyridyl substituted with one methyl and R³ is chloro.

7. The compound of claim 6, wherein the methyl is a meta substituent.

8. The compound of claim 6, wherein the compound is

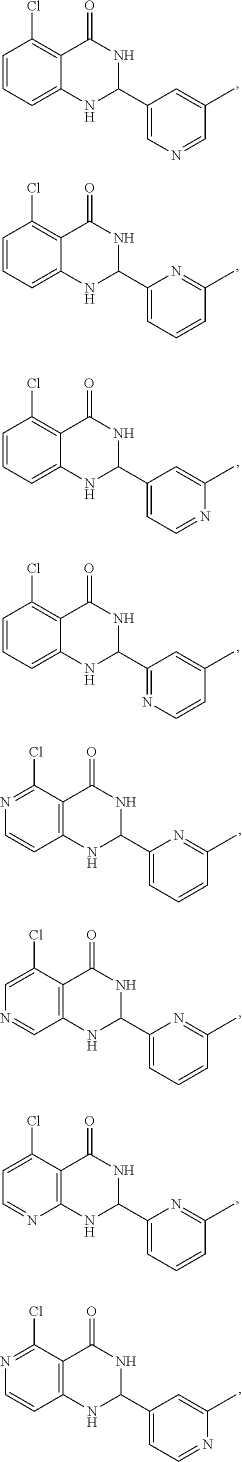

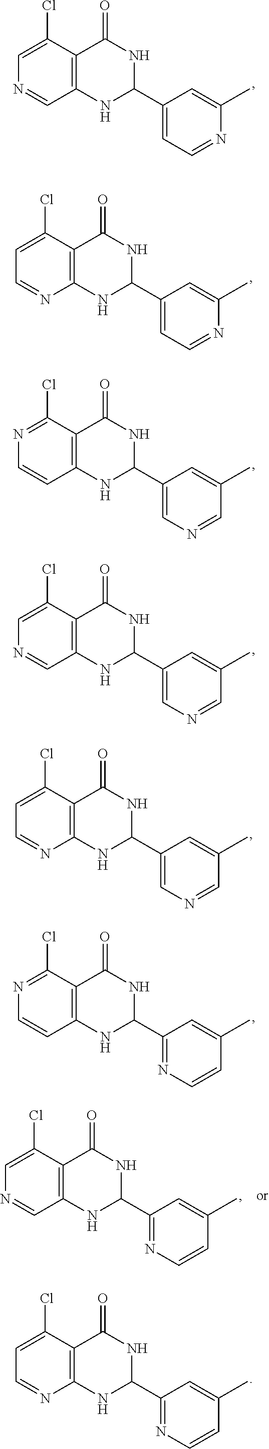

9. The compound of claim 3, wherein the compound is

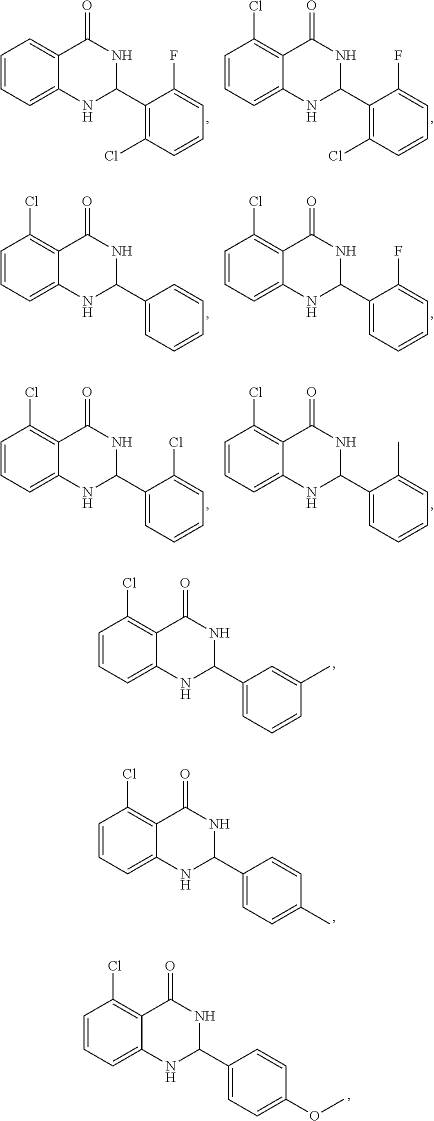

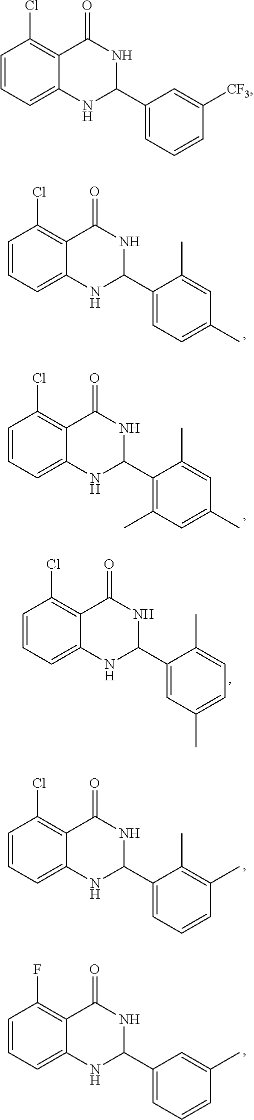

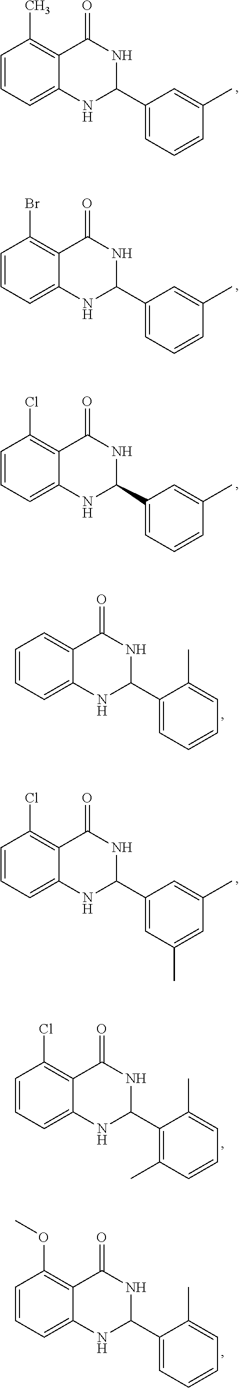

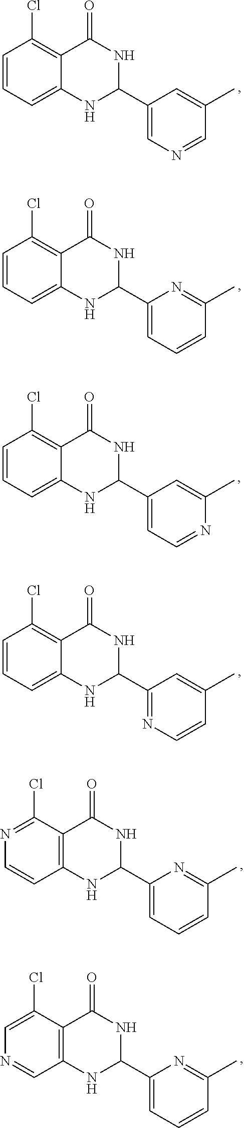 [SMILES: Cc1cncc(C2NC(=O)c3c(Cl)cccc3N2)c1 ; Cc1cccc(C2NC(=O)c3c(Cl)cccc3N2)n1 ; Cc1cc(C2NC(=O)c3c(Cl)cccc3N2)ccn1 ; Cc1ccnc(C2NC(=O)c3c(Cl)cccc3N2)c1 ; Cc1cccc(C2NC(=O)c3c(ccnc3Cl)N2)n1 ; Cc1cccc(C2NC(=O)c3c(Cl)cncc3N2)n1]

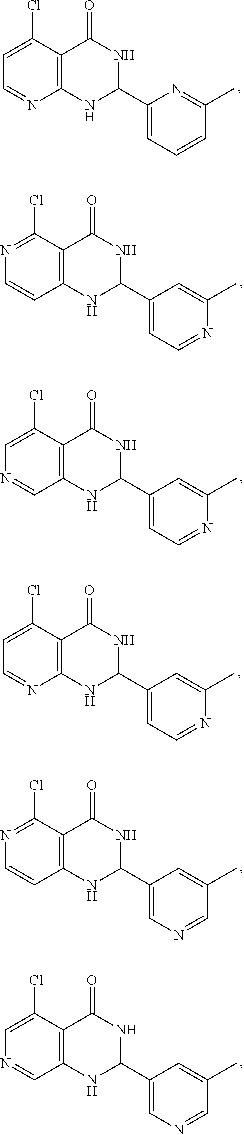 [SMILES: Cc1cccc(C2NC(=O)c3c(Cl)ccnc3N2)n1 ; Cc1cc(C2NC(=O)c3c(ccnc3Cl)N2)ccn1 ; Cc1cc(C2NC(=O)c3c(Cl)cncc3N2)ccn1 ; Cc1cc(C2NC(=O)c3c(Cl)ccnc3N2)ccn1 ; Cc1cncc(C2NC(=O)c3c(ccnc3Cl)N2)c1 ; Cc1cncc(C2NC(=O)c3c(Cl)cncc3N2)c1]

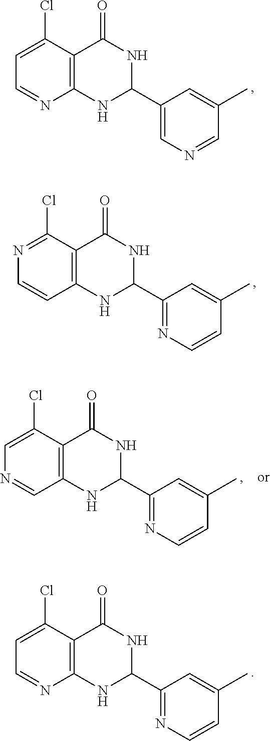 [SMILES: Cc1cncc(C2NC(=O)c3c(Cl)ccnc3N2)c1 ; Cc1ccnc(C2NC(=O)c3c(ccnc3Cl)N2)c1 ; Cc1ccnc(C2NC(=O)c3c(Cl)cncc3N2)c1 ; Cc1ccnc(C2NC(=O)c3c(Cl)ccnc3N2)c1]

10. The compound of claim 2, wherein the compound is

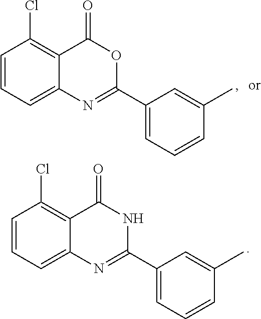 [SMILES: Cc1cccc(-c2nc3cccc(Cl)c3c(=O)o2)c1 ; Cc1cccc(-c2nc3cccc(Cl)c3c(=O)[nH]2)c1]

11. The compound of claim 1, having a formula II(a) 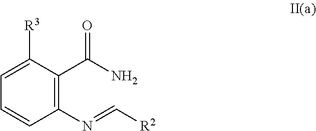
wherein R² is phenyl substituted with one or more halogen; and
R³ is hydrogen or halogen.

12. The compound of claim 11, wherein the compound is

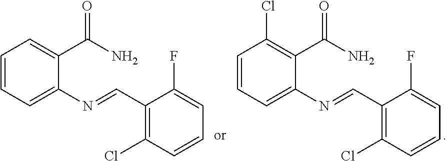

13. The compound of claim 1, having a formula III(a) 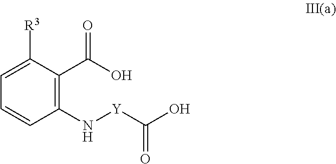
wherein Y is phenylene optionally substituted with halogen; and
R³ is hydrogen or halogen.

14. The compound of claim 13, wherein the compound is

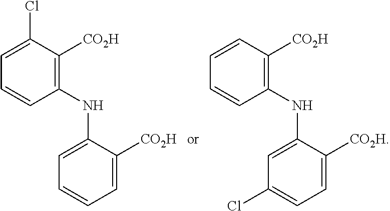 [SMILES: O=C(O)c1ccccc1Nc1cccc(Cl)c1C(=O)O ; O=C(O)c1ccccc1Nc1cc(Cl)ccc1C(=O)O]

15. The compound of claim 1, having a formula IV(a) 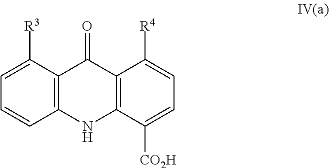
wherein R³ and R⁴ are independently selected from the group consisting of hydrogen and halogen.

16. The compound of claim 15, wherein R³ is chloro and R⁴ is hydrogen.

17. The compound of claim 1, wherein the compound has an IC₅₀ value of from greater than 0 μM to 30 μM for inhibiting activity of polybromo-1 second bromodomain (PBRM1-BD2).

18. A composition comprising the compound of claim 1 and a pharmaceutically acceptable carrier.

19. The composition of claim 18 wherein the composition is formulated to be administered orally.

20. A method of treating cancer in a subject in need thereof, wherein the method comprises administering an effective amount of the compound of claim 1 in order to treat the cancer.

21. The method of claim 20, wherein the cancer is selected from renal cell carcinoma and prostate cancer.

22. The method of claim 20, wherein the compound or the composition is administered in combination with cancer immunotherapy.

23. A method of inhibiting PBRM1-BD in a cell, the method comprising contacting the cell with an effective amount of one or more of any one of the compounds of claim 1.

24. The method of claim 23, wherein the cell is in vivo in a subject in need thereof, the contacting comprising administering an effective amount of the one or more compounds.

25. The method of claim 23, wherein the cell or subject is a cancer cell.

26. The method of claim 25, wherein the cancer cell is a prostate cancer cell.
